(19) United States
(12) Patent Application Publication
(10) Pub. No.: US 2026/0085292 A1
(43) Pub. Date: Mar. 26, 2026

(54) MINIMAL ESSENTIAL MEDIA FOR THE CULTURE OF HUMAN INDUCED PLURIPOTENT STEM CELLS

(71) Applicant: Northwestern University, Evanston, IL (US)

(72) Inventor: Paul Wesley Burridge, Evanston, IL (US)

(21) Appl. No.: 19/109,550

(22) PCT Filed: Sep. 11, 2023
(86) PCT No.: PCT/US2023/073878

§ 371 Date: Mar. 6, 2025

Related U.S. Application Data
(60) Provisional application No. 63/375,164, filed on Sep. 9, 2022.

(51) Int. Cl. C12N 5/074; C12N 5/071; C12N 5/077; C12N 5/0793

(52) U.S. Cl. CPC C12N 5/0696; C12N 5/0619; C12N 5/0657; C12N 5/067; C12N 5/069; C12N 2500/24; C12N 2500/32; C12N 2500/38; C12N 2500/60; C12N 2501/115; C12N 2501/15; C12N 2506/45

(57) ABSTRACT

Disclosed is minimal essential media for culturing induced human pluripotent stem cells (iPSCs), methods for preparing the media, and methods of using the media.

### CROSS-REFERENCE TO RELATED APPLICATIONS

This application claims priority to U.S. Provisional Application No. 63/375,164, filed Sep. 9, 2022, which is incorporated by reference in its entirety.

### STATEMENT REGARDING FEDERALLY SPONSORED RESEARCH

This invention was made with government support under grant numbers CA220002 and CA261898 awarded by the National Institutes of Health. The government has certain rights in the invention.

### BACKGROUND

The first publications demonstrating the isolation and culture of human embryonic stem cells (hESCs) used the basal medium DMEM with 20% fetal bovine serum (FBS) (Thomson et al., 1998) in combination with a ‘feeder layer’ of mouse embryonic fibroblasts (MEFs). This was followed by replacement of DMEM with a low osmolarity variant called Knockout (KO)-DMEM (Amit et al., 2000) before settling on DMEM/F12 with 20% Knockout Serum Replacement (KSR) (Schulz et al., 2004). DMEM/F12 has since formed the basis of nearly all subsequent feeder-free, defined and chemically defined media including TeSR1 (Ludwig et al., 2006), DC-HAIF (Wang et al., 2007), E8 (Chen et al., 2011), FTDA (Frank et al., 2012), i-DEAL (Marinho et al., 2015), AKIT (Yasuda et al., 2018) and our own B8 (Kuo et al., 2020). Few experiments have been completed to compare alternatives to DMEM/F12 for hiPSC culture; in the CDM-BSA formula IMDM/F12 was used and later modified to Advanced DMEM/F12 mixed 50:50 with IMDM (Hannan et al., 2013). One media, hESF9T (Furue et al., 2008; Yamasaki et al., 2014) utilized a complex basal media ‘ESF’ designed for mESCs (Furue et al., 2005) although details of its development were not provided. Limited work has been completed on comparing suitable basal media for chemically defined hESC/hiPSC culture, but existing work has shown similar growth in DMEM/F12 to the comparatively simple MEMα (Chen et al., 2011), potentially suggesting that a complex basal medium might not be essential.

The formula of DMEM/F12 is derived from a 50:50 mix of two existing media which were developed in the 1950 and 60s for mouse fibroblasts (DMEM) and Chinese hamster ovary cells (F12). DMEM is a derivative of the formulation Basal Medium Eagle's (BME) (Eagle, 1955b; c; Eagle et al., 1956). BME was developed with a compromise approach still in use today: the addition of a small percentage of blood serum (either 2% dialyzed horse serum for mouse fibroblasts or 5% dialyzed human serum for HeLa cells) to provide certain undefined but necessary media components. This work demonstrated that 13 amino acids were essential (arginine, cystine, glutamine, histidine, isoleucine, leucine, lysine, methionine, phenylalanine, threonine, tryptophan, tyrosine, and valine) and that 7 amino acids (alanine, aspartic acid, glutamic acid, glycine, hydroxyproline, proline, and serine) were not essential. Later work went on to show that 8 vitamins (choline, folic acid, nicotinamide, pantothenic acid, pyridoxal, riboflavin, thiamine, and myo-inositol) are essential for human cells (Eagle, 1955a; Eagle et al., 1957) and biotin was found to be superfluous. Similar work on the inorganic salts demonstrated clear roles for Na⁺, Cl⁻, K⁺, and H₂PO₄⁻ in Hela cells, along with a low concentration of Ca²⁺ and no (or very low) requirement for Mg²⁺ or HCO₃⁻ (Eagle, 1956). Work on cells in suspension suggested that doubling the concentrations of amino acids was appropriate for high density cultures (Mc et al., 1957) which was adopted and became the MEM formula (Lockart and Eagle, 1959). These MEM concentrations were doubled again and the vitamins quadrupled (without experimental evidence provided) in the DMEM formula (Dulbecco and Freeman, 1959). Of note, commercial versions of DMEM (e.g., Gibco or Corning) are often formulated with additional glycine, iron nitrate, and high glucose (25 mM) not in the original formula. Further work suggested that the addition of non-essential amino acids (NEAAs) and glycine previously not included in the BME/MEM formulations did enhance clonal growth of human cells, especially serine (Lockart and Eagle, 1959).

Contemporaneously, work on the ‘F’ series of media that resulted in F12 was completed, specifically studying single cell growth. This started with numerous revisions of a ‘Nutrient Solution’ supplemented with 30% serum (Cieciura et al, 1956; Puck et al., 1956; Sato et al., 1957) resulting in a serum-free but albumin- and fetuin-containing formula (Fisher et al., 1959). This was followed by studies in CHO cells producing iterations F7, in which hypoxanthine, thymidine, andiron were added (Ham, 1962), F10, in which zinc and copper were also added (Ham, 1963), and finally F12 which added putrescine, linoleic acid, and lipoic acid, and finally eliminated albumin and fetuin (Ham, 1965). In all cases during the development of the F-series media, the formulations were not intended to be minimal media, and multiple components were known to not be required, and no data was provided to explain the rationale or concentration optimization.

As the predecessors of both DMEM and F12 were optimized in the presence of animal-derived serum or albumin and the rationale for the inclusion of many components or their concentrations was not provided, it is unlikely that DMEM/F12 is the optimal formulation for hiPSC growth, especially for chemically defined and weekend-free protocols.

### SUMMARY

One aspect of the disclosure is a media for culturing induced human pluripotent stem cells (iPSCs), wherein the media comprises defined components for culturing iPSCs that include a) a salt solution comprising Calcium chloride (CaCl₂), Iron (II) sulfate heptahydrate (FeSO4·7H2O), Magnesium sulfate (MgSO4), Potassium chloride (KCl), Sodium bicarbonate (NaHCO3), Sodium chloride (NaCl), Sodium phosphate monobasic monohydrate (NaH2PO4·H2O) and Zinc sulfate heptahydrate (ZnSO4·7H2O), b) a vitamin solution comprising Biotin (B7), Choline bitartrate, D-Pantothenic acid hemicalcium salt (B5), Folic acid (B9), Nicotinamide (B3), Pyridoxine hydrochloride (B6), Riboflavin (B2), and myo-Inositol; and c) an amino acid solution comprising Glycine, L-Arginine HCl, L-Cysteine HCl·H2O, L-Glutamine, L-Histidine HCl·H2O, L-Isoleucine, L-Leucine, L-Lysine HCl, L-Methionine, L-Phenylalanine, L-Threonine, L-Tryptophan, L-Tyrosine disodium salt hydrate, and L-Valine.

In some embodiment, the amino acid solution may contain L-Alanine, L-Asparagine·H2O, L-Aspartic acid, L-Glutamic acid, L-Proline and L-Serine. In some embodiments, the salt solution does not contain Copper (II) sulfate pentahydrate (CuSO4·5H2O), Iron (III) nitrate nonahydrate (Fe(NO3)3·9H2O), Magnesium chloride (MgCl2), or Sodium phosphate dibasic (Na2HPO4). In some embodiments, the vitamin solution does not contain choline chloride, Pyridoxal hydrochloride (B6), Thiamine hydrochloride (B1), or Vitamin B12. In some embodiments, the amino acid solution does not comprise L-Cystine 2HCl or L-Histidine.

In some embodiments, the media may not contain one or more of HEPES, Hypoxanthine, Linoleic acid, Lipoic acid, Phenol red, Putrescine dihydrochloride, Thymidine, copper salt, or linoleic acid.

Another aspect is a method for culturing induced pluripotent stem cells (iPSCs) by seeding the iPSCs in the media of the present disclosure, for example the media described above, and culturing the iPSCs under conditions suitable for culturing the iPSCs. In embodiments, the media may or may not be changed during culture of the iPSCs.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Comparison of basal media for hiPSC growth. (A) Concentrations of media formula components previously optimized for B8 with modifications to reduce cost. (B) Concentrations of the major components in DMEM/F12. For all commercial basal media tested, the concentrations of these components was adjusted to match these DMEM/F12 concentrations. (C) Schematic of daily medium exchange schedule. (D) Comparison of hiPSC growth in B8 with various common commercial basal media, using a one-passage, daily medium change assay. Normalized to DMEM/F12. n=9, two hiPSC lines (19c3 and 23c1). None were significantly better than DMEM/F12. (E) Comparison of hiPSC growth in B8 with various common commercial basal media using a three-passage assay. Normalized to DMEM/F12. n=4, two hiPSC lines (19c3 and 23c1). All were significantly worse than DMEM/F12. (F) Heatmap-based comparison of media components found in DMEM/F12 but not in other common media formulations. (G) Comparison of hiPSC growth in our in-house DMEM/F12 formula without trace metals or extras but with the addition individual components at concentrations used in DMEM/F12. Normalized to commercial DMEM/F12, two-passage assay, n=4, two hiPSC lines (19c3 and 23c1). (H) Titration of copper, iron, and zinc concentrations, with daily medium change, two-passage assay, n=4, two hiPSC lines (19c3 and 23c1). Normalized to concentration in DMEM/F12, indicated by a dashed black line. Concentration selected for BMEM is indicated by a dashed blue line. All error bars are SEM, and “n” indicates the number of independent replicates from separate experiments. ns, p>0.05, *p % 0.05, **p % 0.01, ***p % 0.001.

FIG. 2. Titration of amino acid concentrations for hiPSC growth. (A) Titration of essential amino acid concentrations with daily medium change. Normalized to concentration in DMEM/F12, indicated by a dashed black line. Concentration selected for BMEM is indicated by a dashed blue line. Two-passage assay (except for L-glutamine and L-alanyl-L-glutamine, which was a three-passage assay), n=4, two hiPSC lines (19c3 and 23c1). (B) Heatmap-based comparison of essential amino acid concentrations found in common media formulations compared with BMEM (C) Heatmap-based comparison of non-essential amino acid concentrations found in common media formulations compared with BMEM. (D) Addition of individual non-essential amino acid or combination in pairs, triples, quartets, or quintets, with daily medium change. Five-passage assay, n=7-8, two hiPSC lines (19c3 and 23c1). All error bars are SEM, and “n” indicates the number of independent replicates from separate experiments. ns, p>0.05, *p % 0.05.

FIG. 3. Titration of trace metal and essential amino acid concentrations for hiPSC growth using a “no medium change” strategy. (A) Schematic of no medium change schedule; using this method, thiazovivin is always in the medium. (B) Titration of copper, iron, and zinc concentrations, without daily medium change, two-passage assay. n=2, two hiPSC lines (19c3 and 23c1) (C) Titration of essential amino acid concentrations without daily medium change. Two-passage assay (except for L-glutamine and L-alanyl-L-glutamine which was a three-passage assay), n=2-4, two hiPSC lines (19c3 and 23c1). (D) Comparison of growth using a no medium change strategy in media with either 1,000 mM of L-glutamine or 2,000 mM of L-alanyl-L-glutamine, three-passage assay, n=4, two hiPSC lines (19c3 and 23c1). (E) Heatmap of concentrations of L-glutamine or L-alanyl-L-glutamine (GlutaMAX) found in common medium compared with BMEM. Normalized to concentration in DMEM/F12, indicated by a dashed black line. Concentration selected for BMEM is indicated by a dashed blue line. All error bars are SEM, and “n” indicates the number of independent replicates from separate experiments. ns, p>0.05.

FIG. 4. Titration of vitamin, salt, glucose, and pyruvate concentrations for hiPSC growth. Titration experiments with daily medium change, where relative growth is normalized to DMEM/F12 concentration indicated by a dashed black line. Selected BMEM concentrations are indicated by dashed purple lines. (A) Titration of vitamin concentrations. n=4-9, two hiPSC lines (19c3 and 23c1), number of passages required for absence of vitamin to result in complete cell death is indicated, up to five passages (B) Heatmap-based comparison of vitamin concentrations found in common media formulations compared with BMEM. (C) Titration of inorganic salts concentrations. Two-passage assay (except for sodium phosphate which was a five-passage assay), n=4-6, two hiPSC lines (19c3 and 23c1) (D) Titration of sodium phosphate, glucose, and sodium pyruvate concentrations. Two-passage assay, n=12-21, two hiPSC lines. (E) Titration of sodium bicarbonate concentration with pH normalized to 7.1. Two-passage assay, n=12, two hiPSC lines (19c3 and 23c1). (F) Optimization of HEPES concentration pH normalized to 7.1. Two-passage assay, n=7, two hiPSC lines (19c3 and 23c1). (G) Titration of NaCl concentration in the presence of 22,500 mM sodium bicarbonate and its regulation of medium osmolarity. Two-passage assay, n=4, two hiPSC lines (19c3 and 23c1). (H) Heatmap-based comparison of inorganic salts and other component concentrations found in common media formulations compared with BMEM All error bars are SEM, and “n” indicates the number of independent replicates from separate experiments. ns, p>0.05, *p % 0.05, ****p % 0.0001.

FIG. 5. Titration of vitamin, salt, glucose, and pyruvate concentrations for biPSC growth using a “no medium change” strategy. (A) Titration of vitamin concentrations, without medium change. n=2-8, two hiPSC lines (19c3 and 23c1). (B) Titration of thiamine concentrations, with and without medium change when culturing cells in a normoxic (21% O₂) rather than typical hypoxic (5% O₂) environment. n=2-8, two hiPSC lines (19c3 and 23c1). (C) Titration of inorganic salts concentrations. Two-passage assay, n=2, two hiPSC lines (19c3 and 23c1). (D) Titration of sodium phosphate, glucose, and sodium pyruvate concentrations. Two-passage assay (except for sodium phosphate which was a five-passage assay), n=2-10, two hiPSC lines (19c3 and 23c1). (E) Titration of sodium bicarbonate, HEPES, and NaCl, with no medium change. Two-passage assay, n=2-3, two hiPSC lines (19c3 and 23c1). (F) Assessment of pH value changes in culture conditions without cells. After initial pH measurement, values were normalized to 7.1 and medium was incubated at 37° C. with 5% CO2. All error bars are SEM. All values are normalized to the concentration in DMEM/F12, indicated by a dashed black line. Concentration selected for BMEM is indicated by a dashed blue line. All error bars are SEM, and “n” indicates the number of independent replicates from separate experiments. ns, p>0.05, *p % 0.05, **p % 0.01, ***p % 0.001, ****p % 0.0001.

FIG. 6. Comparison gene expression in hiPSCs after culture in BMEM or DMEM/F12. (A) Hierarchical clustering of 250 most variable genes. (B) Principal-component analysis for the 30 samples, individual lines are encircled. (C) Volcano plot with undifferentiated state-affiliated genes labeled; up, higher expression in BMEM. (D) Comparative expression of undifferentiated state-affiliated genes in BMEM vs. DMEM/F12. Genes associated with the primed state are marked in blue and POU5F1, NANOG, and SOX2 are marked in dark gray. (E and F) Transcripts per million for POU5F1 (OCT4) expression (E) and NANOG (F) showing consistent higher expression in BMEM. All error bars are SEM from 15 conditions/cell lines.

FIG. 7. Comparison of hiPSCs cultured and/or derived in BMEM or DMEM/F12. (A) Undifferentiated cell markers for are similarly expressed in hiPSC cell lines 19c3, 23c1, and 22c10 cultured in DMEM/F12 or BMEM (>25 passages). (B) BMEM-cultured hiPSCs (>25 passages) are equally capable of differentiating into cardiomyocytes, endothelial, vascular smooth muscle cells, neural, and hepatocyte-like cells. (C) Comparison of growth when using BMEM with daily medium change (with thiazovivin for the first 24 h), BMEM without medium change (which has thiazovivin throughout) or DMEM/F12 with daily medium change (with thiazovivin for the first 24 h) over 44 passages, two hiPSC lines (19c3 and 23c1) 19c3 were kept in culture from passage 61 (after 31 passages in BMEM) to passage 105. 23c1 were kept in culture from passage 75 (after 45 passages in BMEM) to passage 119. (D) Undifferentiated cell marker expression in nine hiPSC lines derived in BMEM at >p37. (E) Immunocytochemistry analysis of seven hiPSC lines derived in BMEM at >p37. Scale bars, 100 mm. (F) Phase contrast images of hiPSC lines cultured in DMEM/F12 or BMEM (19c3 [28 passages in BMEM] and 23c1 [40 passages in BMEM]), and hiPSC lines both derived and cultured in BMEM (5054, 3198, and 4682). Scale bars, 100 mm. (G) Karyotyping for hiPSCs cultured in BMEM for >30 passages (19c3), and derived in BMEM (5054 and 3198) compared with standard culture conditions in DMEM/F12 (19c3). Each dot indicates an independent replicate from a separate experiment (n=3-12) All error bars are SEM.

FIG. 8. Titration of NEAAs for hiPSC growth and analyses using a ‘no medium change’ strategy, related to FIG. 2. Titration of NEAA concentrations using 2-passage assay and an additive approach with daily (A) or without (B) medium change. n=2-4, 2 hiPSC lines (19c3 and 23c1). (C) Addition of individual non-essential amino acid or combination in pairs, triples, quartets, or quintets, without daily medium change. 5-passage assay, n=9-10, 2 hiPSC lines (19c3 and 23c1). All error bars are s.e.m., and “n” indicates the number of independent replicates from separate experiments. (D) Comparison of the requirement for NEAAs with daily media change when culturing cells in a normoxic (21% (2) rather than typical hypoxic (5% (2) environment. 5-passage assay, n=6, 2 hiPSC lines (19c3 and 23c1). ns indicates P≥0.05, * indicates P≤0.05, ** indicates P≤0.01, *** indicates P≤0.001, **** indicates P≤0.0001.

FIG. 9. Metabolomic analysis of intracellular metabolites in BMEM- and DMEM/F12-cultured hiPSC, related to FIGS. 2 and 3. Fold change heat map for top 25 intracellular metabolites in BMEM and DMEM/F12-cultured hiPSC with (A) or without (B) daily medium change. Volcano plot for intracellular metabolites with (C) and without (D) daily medium change, up=higher values in BMEM. Enrichment analysis of metabolite sets of BMEM versus DMEM/F12-cultured hiPSCs with (E) and without (F) daily medium change. n=4, 2 hiPSC lines (19c3 and 23c1).

FIG. 10. Metabolomic analysis of spent media from BMEM- and DMEM/F12-cultured hiPSC, related to FIGS. 2 and 3. Top 15 common intracellular metabolites by normalized peak area for BMEM- and DMEM/F12-cultured hiPSC with (A) or without (B) daily medium change. n=4, 2 hiPSC lines (19c3 and 23c1). (C) Normalized peak area for the 6 non-essential amino acids for hiPSC's cultured in BMEM with and without non-essential amino acids. n=2 for each hiPSC line (19c3 and 23c1). All error bars are s.e.m., and “n” indicates the number of independent replicates from separate experiments. ns indicates P>0.05, * indicates P≤0.05, ** indicates P≤0.01, *** indicates P≤0.001, **** indicates P≤0.0001.

FIG. 11. Results from Qiagen Ingenuity Pathway Analysis, related to FIG. 6.

FIG. 12. Comparison of expression of genes associated with metabolism in hiPSC after culture in BMEM or DMEM/F12, related to FIG. 6. (A) Demonstration of higher relative expression of glycolysis-related genes in BMEM vs. DMEM/F12. (B) Demonstration of lower relative expression of mitochondrial metabolism-related genes in BMEM vs. DMEM/F12. Expression (transcripts per million) of glycolysis-related genes PFKFB3 (6-phosphofructo-2-kinase fructose-2,6-biphosphatase 3) (C), SLC2A3 (glucose transporter 3) (D), and mitochondrial-related genes PDHB (pyruvate dehydrogenase E1 subunit beta) (E) and OGDH (alpha-ketoglutarate dehydrogenase) (F) in BMEM vs. DMEM/F12 for the 30 analyzed samples. All error bars are s.e.m.

FIG. 13. Assessment of hiPSC growth in BMEM on different extracellular matrices, related to characterization of BMEM-cultured hiPSC lines on commercially available extracellular matrices. (A) Comparison of hiPSC growth in BMEM on different commercially available matrices, 5 passage assay, n=6, 2 hiPSC lines (19c3 and 23c1), differences were not significant. (B) Undifferentiated state affiliated marker expression for hiPSC lines cultured in BMEM on six commercially available matrices. n=2-4, 2 hiPSC lines (19c3 and 23c1), differences were not significant. All error bars are s.e.m., and “n” indicates the number of independent replicates from separate experiments. (C) Phase contrast images of hiPSC lines cultured in BMEM 19c3 (28 passages in BMEM) and 23c1 (30 passages in BMEM), on 6 commercially available ECMs. Scale bar=100 μm.

FIG. 14. Assessment of commercial DMEM/F12 media, related to the Discussion section. Reported pH and osmolarity values in the certificates of analysis of a selection of commercial DMEM/F12 lots and suppliers.

### DETAILED DESCRIPTION

The nutritional requirements for human induced pluripotent stem cell (hiPSC) growth have not been extensively studied. Here, the inventors develop a simplified basal medium consisting of essential components, demonstrating that many ingredients of DMEM/F12 are either not essential or are at suboptimal concentrations. This new basal medium or basal minimal essential medium, called BMEM, enhances the growth rate of hiPSCs over DMEM/F12-based media, supports derivation of multiple hiPSC lines, and allows differentiation to multiple lineages. hiPSCs cultured in BMEM consistently have enhanced expression of undifferentiated cell markers such as POU5F1 and NANOG, along with increased expression of markers of the primed state and reduced expression of markers of the naive state. This work describes titration of the nutritional requirements of human pluripotent cell culture and identifies that suitable nutrition enhances the pluripotent state. One example of the described basal minimal essential medium has just 39 components. A minimal basal medium formula is described that promotes growth of hiPSC and methods for preparing and using the same.

The terms “cell culture medium,” “cell culture media,” and “culture medium” refer to the solutions used for growing, storing, handling and maintaining cells and cell lines. Such solutions generally include various factors necessary for cell attachment, growth, and maintenance of the cellular environment. For example, a typical solution may include a basal media formulation, various supplements depending on the cell type and, occasionally, antibiotics. In some embodiments, a solution may include at least one component from one or more of the following categories: 1) an energy source, usually in the form of a carbohydrate such as glucose; 2) all essential amino acids, and usually the basic set of twenty amino acids plus cystine; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The solution may optionally be supplemented with one or more components from any of the following categories: 1) hormones and other growth factors as, for example, insulin, transferrin, and epidermal growth factor; 2) salts and buffers as, for example, calcium, magnesium, phosphate, Tris, HEPES, and sodium bicarbonate; 3) nucleosides and bases such as, for example, adenosine and thymidine, hypoxanthine; and 4) protein and tissue hydrolysates. In general, any suitable cell culture medium may be used. The medium may be comprised of serum, e.g. fetal bovine serum, calf serum or the like. Alternatively, the medium may be serum free, animal free, or protein free.

### Media Components

The term “cell culture media component” or “cell culture component” refers to any heterologous proteins, small molecules, minerals, amino acids, vitamins, salts or other chemical element used as a part of or in the making of for cell culture media. Cell culture components make up the cell culture media. The term “cell culture media ingredient” includes cell culture media components, proteins, peptides, hormones, carbohydrates, amino acids, lipids, vitamins, antibiotics, organic and inorganic salts.

Components of the media may include those listed in Table 1 and any other components listed herein. Additional components may comprise cell culture media supplements, antibiotics, serum or sera, trace elements, hormones, attachment factors, carbohydrates, growth factors, 2-mercaptoethanol, and cytokines.

The term “cell culture media supplement” refers to a combination of one or multiple cell culture media components, with or without other ingredients, for addition to cell culture media. In some embodiments, a cell culture supplement may comprise additional cell culture components or ingredients. In some embodiments, these may comprise insulin, salt, transferrin and growth factors. The growth factors may comprise fibroblast growth factor (FGF), neuregulin-1, brain-derived neurotrophic factor, vascular endothelial cell growth factor, epidermal growth factor, interleukin 2, granulocyte macrophage colony-stimulating factor, transforming growth factor-β1, insulin-like growth factor-I, tumor necrosis factor-α and nerve growth factor.

In some embodiments, the disclosed media may comprise BMEM supplement. The BMEM supplement may comprise recombinant or nonrecombinant human insulin, L-ascorbic acid 2-phosphate trisodium salt, recombinant or nonrecombinant human transferrin, sodium selenite, recombinant or nonrecombinant human FGF2-G3, recombinant or nonrecombinant human TGFb3, or recombinant or nonrecombinant human NRG1. In some embodiments the supplement may comprise the media (“B8”) described in U.S. Patent Publication 20210087525.

The term “minimal” refers to the minimum number of components, at minimal concentration without comprising growth, reprogramming or differentiation.

The disclosed culture medium for use in culturing human induced pluripotent stem cells may comprise one or more of the components described herein, in any and all combination which allow for the culture of human induced pluripotent stem cells. Described herein is a cell culture media consisting of salts, vitamins, amino acids, and recombinant growth factors.

The disclosed media may be used for culturing human induced pluripotent stem cell (hiPSC) growth. hiPSCs may be derived from a direct reprogramming of human somatic cells to a pluripotent stage through ectopic expression of specific transcription factors. hiPSCs may be derived by any means know in the art, including as described herein from peripheral blood with known growth and reprograming factors.

In some embodiments, the disclosed media is useful for culturing a variety of cell types, including, but not limited to embryonic stem cells, adult stem cells, hematopoietic stem cells, organoids, and 3-D cultures.

Components are described for the media for culturing induced human pluripotent stem cells (iPSCs). In some embodiments, the media comprises a salt solution, a vitamin solution, and an amino acid solution. In embodiments, the media comprises a salt solution, a vitamin solution, and an amino acid solution. In some embodiments, the components are 25× salt solution (× solution diluted from stock or purchased solution as indicated in the Methods), 200× vitamin solution, and a 200× amino acid solution, and ultra-pure water.

In some embodiments the salt solution comprises Calcium chloride (CaCl2), Iron (II) sulfate heptahydrate (FeSO4·7H2O), Magnesium sulfate (MgSO4), Potassium chloride (KCl), Sodium bicarbonate (NaHCO3), Sodium chloride (NaCl), Sodium phosphate monobasic monohydrate (NaH2PO4·H2O), and Zinc sulfate heptahydrate (ZnSO4·7H2O). In some embodiments the salt solution does not comprise Copper (II) sulfate pentahydrate (CuSO4·5H2O), Iron (III) nitrate nonahydrate (Fe(NO3)3·9H2O), Magnesium chloride (MgCl2), or Sodium phosphate dibasic (Na2HPO4).

In some embodiments, the iron salt is Iron(II) sulfate heptahydrate (FeSO4·7H2O) and present in the media at a concentration of about 0.1-0.3 uM. In some embodiments, the media comprises zinc salt and the zinc salt is present in the media at a concentration of about 0.1-0.3 uM. In some embodiments, the media does not comprise linoleic acid.

In some embodiments, the vitamin solution comprises Biotin (B7), Choline bitartrate, D-Pantothenic acid hemicalcium salt (B5), Folic acid (B9), Nicotinamide (B3), Pyridoxine hydrochloride (B6), Riboflavin (B2), and myo-Inositol. In some embodiments, the vitamin solution does not comprise choline chloride, Pyridoxal hydrochloride (B6), Thiamine hydrochloride (B1) or Vitamin B12.

In some embodiments, biotin is present in the media at a concentration of about 0.001-01 uM, folic acid is present in the media at a concentration of about 0.05-5 uM, nicotinamide is present in the media at a concentration of about 0.01-5 uM, and pyridoxine is present in the media at a concentration of about 0.05-10 uM.

In some embodiments, the amino acid solution comprises Glycine, L-Arginine HCl, L-Cysteine HCl·H2O, L-Glutamine, L-Histidine HCl·H2O, L-Isoleucine, L-Leucine, L-Lysine HCl, L-Methionine, L-Phenylalanine, L-Threonine, L-Tryptophan, L-Tyrosine disodium salt hydrate, and L-Valine. In some embodiments the amino acid solution does not comprise L-Cystine 2HCl or L-Histidine.

In some embodiments the amino acid solution further comprises nonessential amino acids (NEAA). NEAA include alanine, arginine, asparagine, aspartic acid, cysteine, glutamic acid, glutamine, glycine, proline, serine, and tyrosine. In some embodiments the media described herein further comprises NEAA. In some embodiments the media further comprises L-Alanine, L-Asparagine·H2O, L-Aspartic acid, L-Glutamic acid, L-Proline and L-Serine.

In some embodiments, the concentration of L-Arginine is present in the media at a concentration of about 150-250 uM, L-tryptophan is present in the media at a concentration of about 150-250 uM, and L-glutamine is present in the media at a concentration of about 500-1500 uM.

The disclosed media may comprise a salt solution, vitamin solution, an amino acid solution, D-(+)-Glucose, Phenol red sodium salt, and sodium pyruvate. In some embodiments, the media may not comprise HEPES, Hypoxanthine, Linoleic acid, Lipoic acid, Phenol red, Putrescine dihydrochloride, or Thymidine.

In some embodiments the disclosed media comprises calcium salt wherein the calcium salt is CaCl₂) is present at a concentration of about 350-650 uM. In some embodiments, the media comprises pyruvate salt, wherein the pyruvate salt is Sodium pyruvate is present in the media at a concentration of about 600-900 uM.

The disclosed media may comprise or consist of the following components: (a) amino Acids comprising or consisting of: Glycine; L-Alanine; L-Arginine; L-Asparagine; L-Cysteine; L-Glutamic Acid; L-Glutamine; L-Histidine; L-Isoleucine; L-Lysine; L-Methionine; L-Phenylalanine; L-Threonine; L-Tryptophan, L-Tyrosine; and L-Valine; (b) vitamins comprising or consisting of: Biotin (B7); Choline; D-Pantothenic acid (B5); Folic acid (B9); Nicotinamide (B3); Pyridoxine (B6), Riboflavin (B2); Vitamin B12; and myo-Inositol: (c) inorganic salts comprising or consisting of: Calcium chloride, Iron (II) sulfate heptahydrate; Magnesium sulfate; Potassium chloride; Sodium bicarbonate; Sodium chloride; Sodium phosphate monobasic monohydrate; and Zinc sulfate heptahydrate; and (d) other components comprising or consisting of: D-(+)-glucose; phenol red sodium salt; and sodium pyruvate.

The media described herein may comprise a buffer or buffering components or a buffering system. A cell culture media buffering system helps to ensure that the media will be somewhat resistant to change in pH. The media of the present disclosure may have a pH in the range of 7.1 to 7.2. The buffering system of the media described herein may comprise sodium bicarbonate. The sodium bicarbonate may be present at a concentration range of about 20 mM to about 30 mM. In some embodiments, the sodium biocarbonate is present at approximately 22.5 mM when the pH of the media is 7.1.

Maintenance of physiological osmolarity is essential for cell culture and NaCl makes up greater than 50% of the mass of typical powdered media. The media described herein may comprise an osmolarity of about 285-315 mOsm/L. In some embodiments, approximately 110 mM NaCl is suitable to produce an osmolarity of 295 mOsm/L.

In some embodiments the disclosed media comprises the following components. Glycine (optionally at a concentration of about 150-250 uM); L-Alanine (optionally at a concentration of about 35-65 uM); L-Arginine (optionally at a concentration of about 150-250 uM); L-Asparagine (optionally at a concentration of about 35-65 uM); L-Cysteine (optionally at a concentration of about 75-125 uM); L-Glutamic Acid (optionally at a concentration of about 35-65 uM); L-Glutamine (optionally at a concentration of about 800-1200 uM); L-Histidine (optionally at a concentration of about 75-125 uM); L-Isoleucine (optionally at a concentration of about 150-250 uM); L-Leucine (optionally at a concentration of about 350-650 uM); L-Lysine (optionally at a concentration of about 350-650 uM); L-Methionine (optionally at a concentration of about 75-125 uM); L-Phenylalanine (optionally at a concentration of about 75-125 uM); L-Threonine (optionally at a concentration of about 350-650 uM); L-Tryptophan (optionally at a concentration of about 150-250 uM); L-Tyrosine (optionally at a concentration of about 150-250 uM); and L-Valine (optionally at a concentration of about 150-250 uM).

### Methods

A second aspect of the present disclosure provides a method for preparing and using the media of any described herein. In some embodiments, the method comprises dissolving one or more of the components of the media in an aqueous solution.

The components of the media described herein may be combined in an order By way of example, and not limitation, the salt solution, vitamin solution and amino acid solution may be combined separately or together, and with any supplement.

Cell culture media components, ingredients or supplements may be dissolved in a media solution such that they become incorporated into the liquid to form a solution. In some embodiments, the components, ingredients, or supplements are dissolved in water, such as ultra-pure water. In some embodiments, the pH and osmolarity are tested and adjusted after making the media. In some embodiments, the media solution may be sterilized Media may be sterilized by any means known in the art and may include, but is not limited to filtration, heating, or autoclaving. In some embodiments, the pH is tested and adjusted after making the media.

The media described herein may be used to culture cells. The media may or may not be changed daily as the cells are in culture. The media may also be changed every 1, 2, 3, 4, 5, 6, 7, 8, 9, or 10 days as the cells grow in culture. The media may be changed when the cells in culture reach confluence. The media may be changed when the cells are 50%, 60%, 70%, 80%, 90% or any amount in-between, confluent. The media may or may not be changed when the cells in culture are passaged. Passaging is the procedure of harvesting cells from a culture, transferring the cells to one or more culture vessels with or without fresh growth medium, and using those cells to start new cultures. Passaging is also referred to as subculturing.

In some embodiments, the media may comprise different components depending on the frequency of passaging the cells. By way of example, but not limitation, the media may comprise about 1-3 mM sodium phosphate monobasic and may not comprise glycine when media is not changed daily for culture or prior to differentiation. If media is changed daily for culture, it may comprise NEAA, and may not comprise sodium phosphate monobasic.

Induced pluripotent stem cells (also known as iPS cells or iPSCs) are a type of pluripotent stem cell that can be generated directly from a somatic cell. iPSC are typically derived from skin or blood cells that have been reprogrammed back into an embryonic-like pluripotent state that enables the development of an unlimited source of any type of human cell needed for therapeutic purposes. iPSC can be expanded indefinitely, and they are capable to differentiate in all the derivatives of the three germ layers.

In some embodiments, a method for culturing iPSCs comprising seeding or placing the iPSCs in the disclosed media is provided. In some embodiment, the method provides for culturing the iPSCs under conditions suitable for culturing the iPSCs, and optionally changing the media during culture and/or passaging the iPSCs. In some embodiments, the media is not changed during culture.

In some embodiments, according to the method described herein, the cultured iPSCs express one or more markers at a higher level than iPSCs cultured in Dulbecco's modified Eagle Media (DMEM). In some embodiments the markers comprise, KDR, SPRY4, GDF3. NODAL, NANOG, POU5F1, TDGF1, and ZIC2. In some embodiments, the markers comprise DUSP6, ZDHHC22, NEFM, THY1, FAT3, STC1, KLHL4, PLA2G3, PTPRZ1, and SOX11. In some embodiments, the markers comprise SSEA4, TRA-1-60, and TRA-1-81. These markers may also comprise markers associated with metabolism in iPSCs including but not limited to glycolysis-related genes such as 6-phosphofructo-2-kinase/fructose-2,6-biphosphatase 3 (PFKFB3) and glucose transporter 3 (SLC2A3) and mitochondrial-related genes such as pyruvate dehydrogenase E1 subunit beta (PDHB) and alpha-ketoglutarate dehydrogenase (OGDH).

In some embodiments, according to the method described herein, the cultured iPSCs are further differentiated into a more mature or other cell types. The disclosed media may be used to differentiate iPSCs into any cell type. Some cell types comprise mesodermal cells such as hematopoietic, endothelial cell, cardiomyocyte, smooth muscle cell, skeletal muscle cell, renal cell, adipocyte, chondrocyte and osteocytes; ectodermal cells such as neuron, astrocyte, oligodendrocyte, retinal epithelial cell (RPE), epidermal, hair and keratinocytes; and endodermal cells such as hepatocyte, pancreatic β-islet cell, intestinal epithelial cell, and lung alveolar ells. In some embodiments the media described herein can be used to culture iPSC and differentiate them into cardiomyocytes, endothelial, vascular smooth muscle, neural and hepatocyte-like cells.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately.” “substantially,” and “significantly” will be understood by persons of ordinary skill in the art and will vary to some extent on the context in which they are used. If there are uses of the term which are not clear to persons of ordinary skill in the art given the context in which it is used. “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### Examples

Reference is made to the manuscript: Lyra-Leite et al. Stem Cell Reports 2023, “Nutritional requirements of human induced pluripotent stem cells,” the content of which is incorporated herein by reference in its entirety

The nutritional requirements for human induced pluripotent stem cell (hiPSC) growth have not been extensively studied. Here, the inventors develop a simplified basal medium consisting of just 39 components, demonstrating that many ingredients of DMEM/F12 are either not essential or are at suboptimal concentrations. This new basal medium along with the supplement, called BMEM, enhances the growth rate of hiPSCs over DMEM/F12-based media, supports derivation of multiple hiPSC lines, and allows differentiation to multiple lineages. hiPSCs cultured in BMEM consistently have enhanced expression of undifferentiated cell markers such as POU5F1 and NANOG, along with increased expression of markers of the primed state and reduced expression of markers of the naive state. This work describes titration of the nutritional requirements of human pluripotent cell culture and identifies that suitable nutrition enhances the pluripotent state.

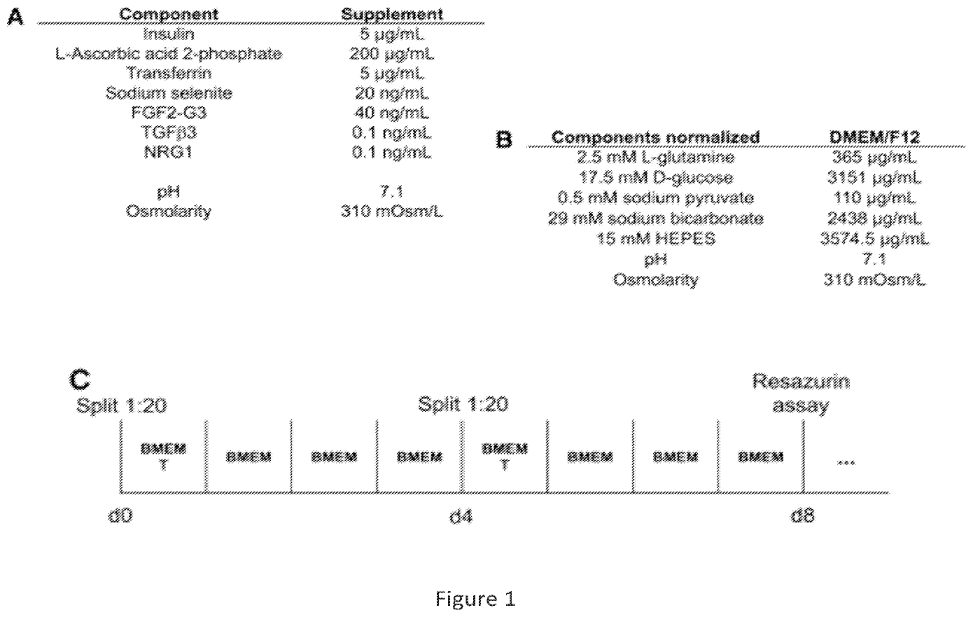

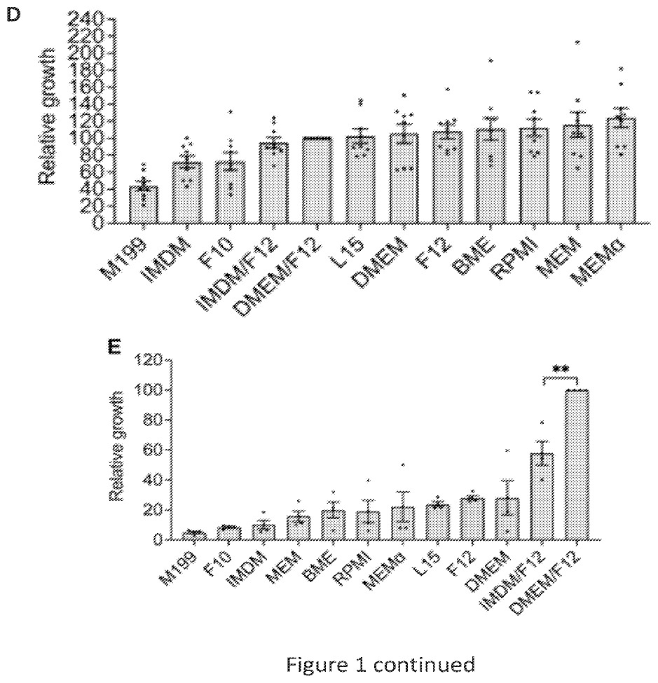

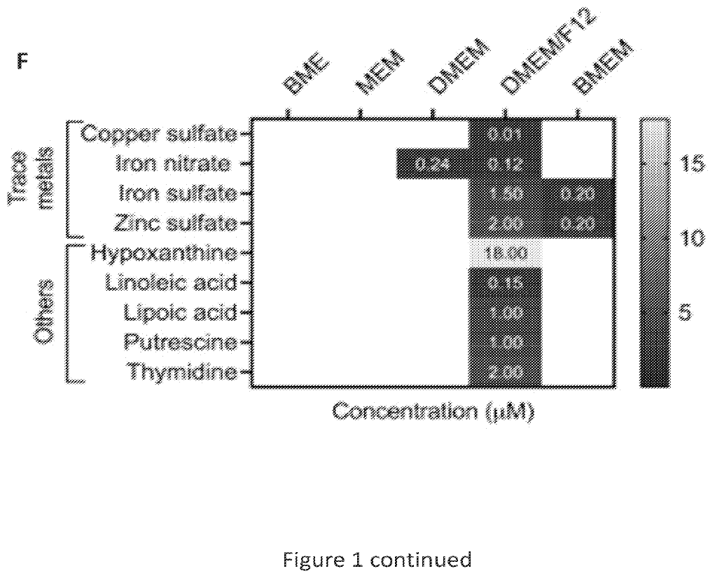

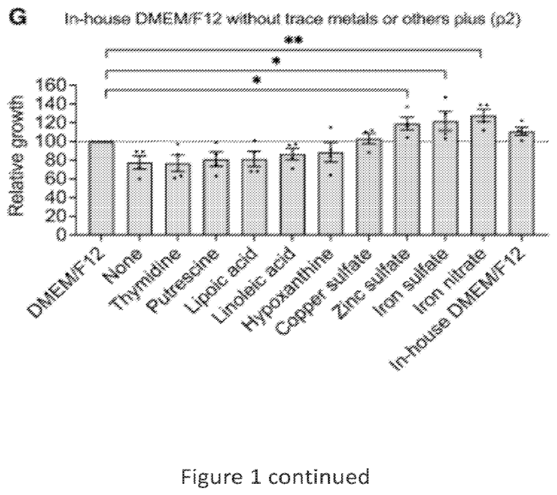

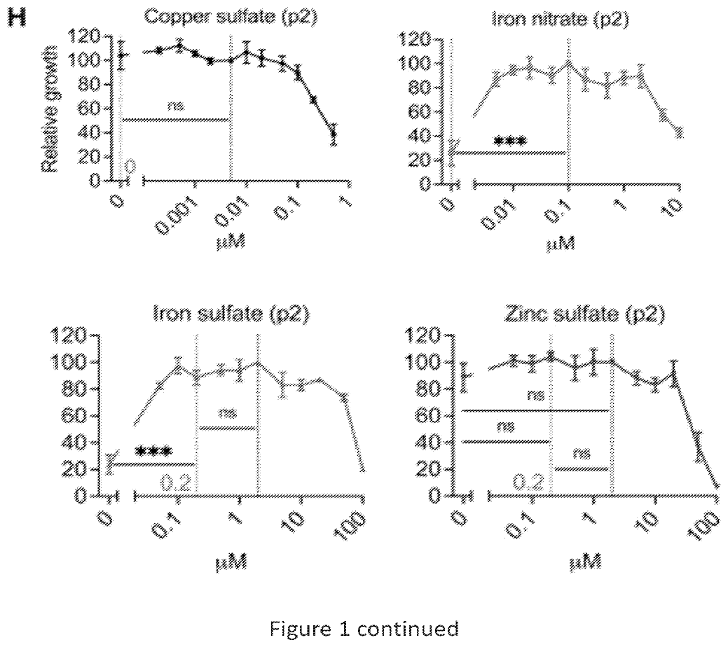

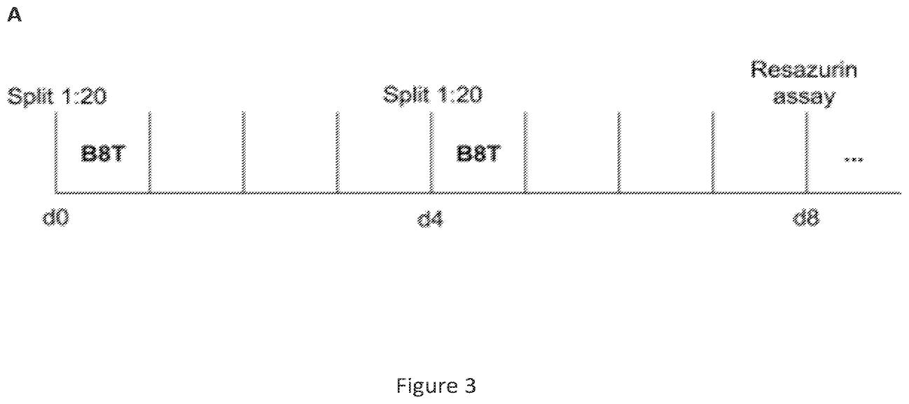

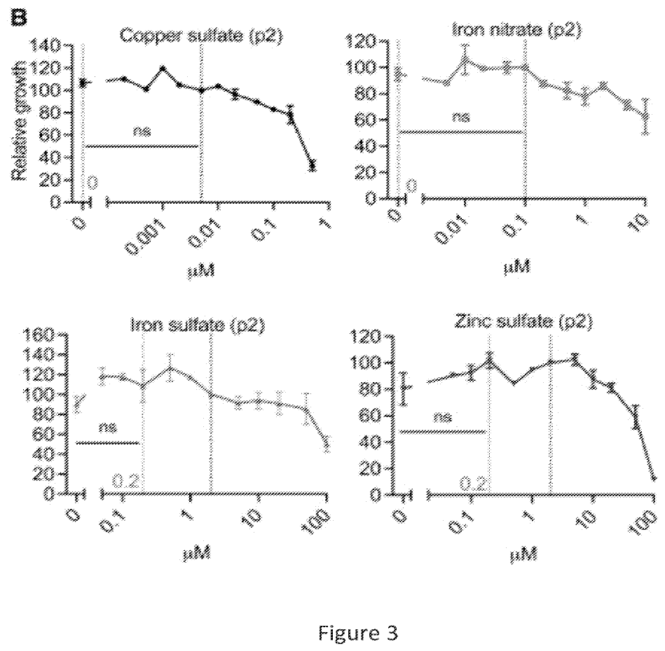

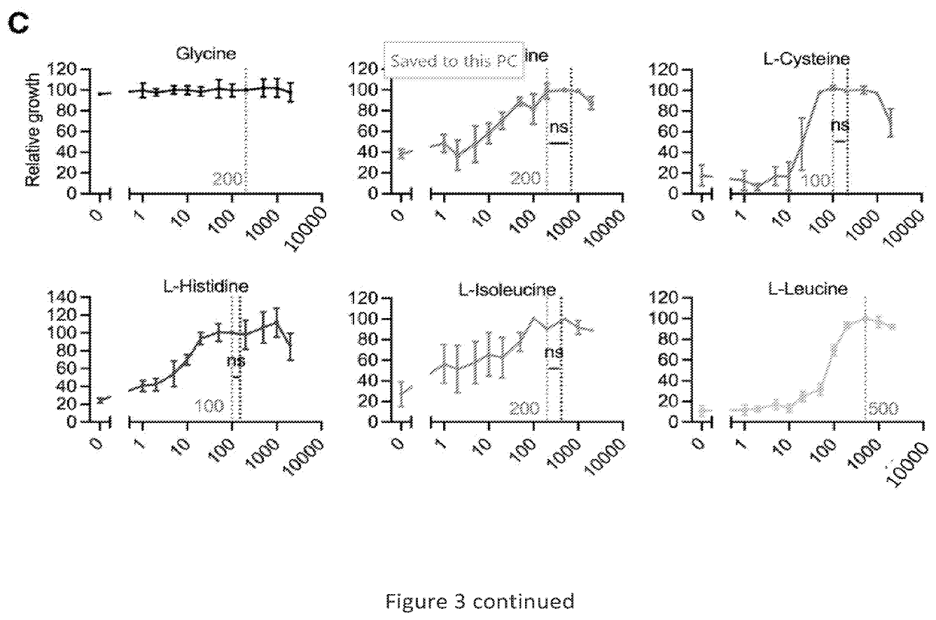

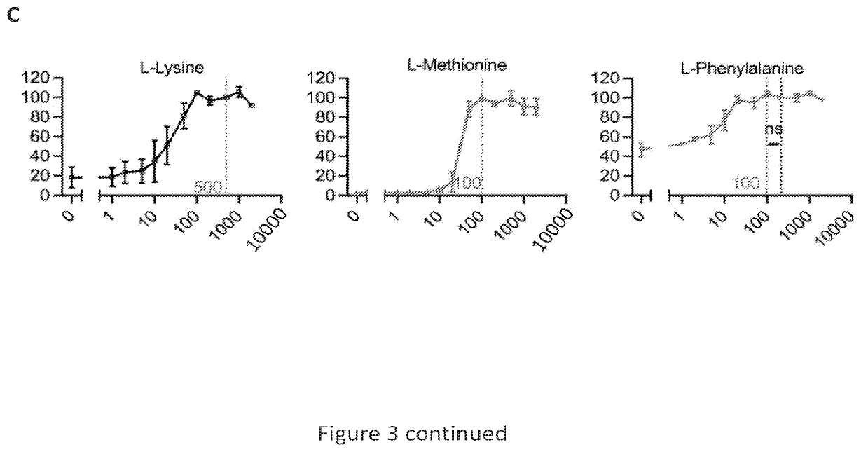

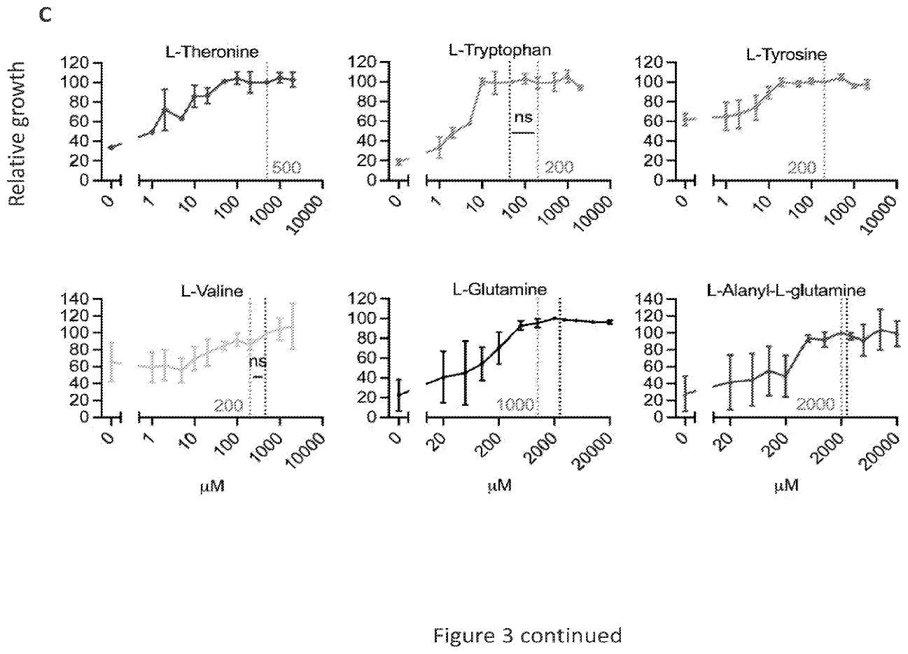

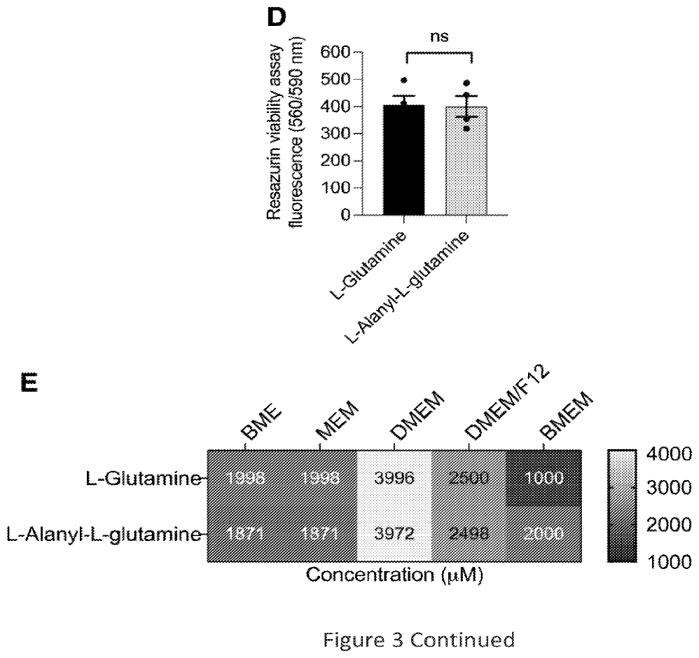

The inventors prior work established “B8” medium consisting of DMEM/F12 and a supplement containing insulin, L-ascorbic acid 2-phosphate, transferrin, selenite, FGF2-G3, TGFb3, and NRG1 as well as demonstrating that a pH of 7.1 and osmolarity of 310 mOsm/L was suitable for hiPSC growth (Kuo et al., 2020), and this supplement has since been further modified (FIG. 1A). Studies for alternatives to the basal medium DMEM/F12 began by assessing the suitability of 12 common commercial media formulations: M199, BME, MEM, MEMa, DMEM, IMDM, RPMI 1640, F10, F12, L15, IMDM/F12, and DMEM/F12. Each commercial medium was normalized to the DMEM/F12 levels of L-glutamine, D-glucose, sodium pyruvate, sodium bicarbonate, pH, and osmolarity (FIG. 1B). To assess the effect of basal medium on hiPSC growth, an assay was developed consisting of plating cells in 12-well plates, and growing cells for 4 days with daily medium changes, before passaging at a 1:20 ratio. This was completed for 1-5 passages followed by a cost-effective resazurin viability assay (FIG. 1C). The number of passages required for each titration assay was determined as the number required for the control “0 mM” concentration to have no surviving cells, up to a maximum of five passages. This was chosen to minimize the effect of variance caused by repeated manual passaging and replating. At least two hiPSC lines were used in parallel throughout the titration process. Identified adjusted concentrations of each group, such as amino acids, was incorporated prior to beginning the next group, such as vitamins, to bring subsequent titrations as close to final formulation as possible. Once the initial full cycle of titration of all components was complete, a second full cycle of was performed to confirm the results in the final formulation. We also completed identical experiments without daily medium change to identify components and concentrations that support this protocol (FIGS. 3, 5, and 8), and to allow for development of a simplified hiPSC culture strategy.

Using the common one-passage assay. MEM and MEMa were found to be the most suitable formulations, in agreement with previous work (Chen et al., 2011) (FIG. 1D). In contrast, when using a more representative three-passage assay, only DMEM/F12 was able to maintain a suitable grown rate (FIG. 1E). This suggests that a more complex multi-passage assay is required to establish the basal medium requirements as some components take longer than 4 days to deplete, hence this method was used for subsequent experiments.

### Low-Molecular-Weight Nutrients

To establish the medium component requirements for hiPSC growth, attention was directed to the more unique components of DMEM/F12 including the trace metals copper sulfate, iron nitrate, iron sulfate, and zinc sulfate; and the group defined as “others” that consists of hypoxanthine, linoleic acid, lipoic acid, putrescine, and thymidine (FIG. 1F). An in-house formulation was developed of DMEM/F12 without any of these components, and their individual addition was tested (FIG. 1G). Using a two-passage assay, a 4-log concentration titration was completed to identify the suitable concentrations for each of these trace metals (FIG. 1H) and identical experiments without medium change (FIG. 3A). No advantage of the addition of copper to the medium was found, and it was toxic at higher concentrations (>0.1 mM). Iron is indispensable for respiration and metabolism and was shown to be essential, with either the nitrate or sulfate forms suitable, although both forms were toxic at high concentrations. Zinc, an essential element involved in insulin signaling, was not shown to be essential but did improve growth by approximately 10% and was therefore included. Concentrations of iron sulfate and zinc sulfate were conservatively selected that were one dose above the lowest concentration that resulted in the highest growth for both the daily and no medium change data. All other components, hypoxanthine, linoleic acid, lipoic acid, putrescine, and thymidine, were found to be superfluous, suggesting that hiPSCs have no nutritional need for these components (FIG. 1G).

### Amino Acids and Metabolomics

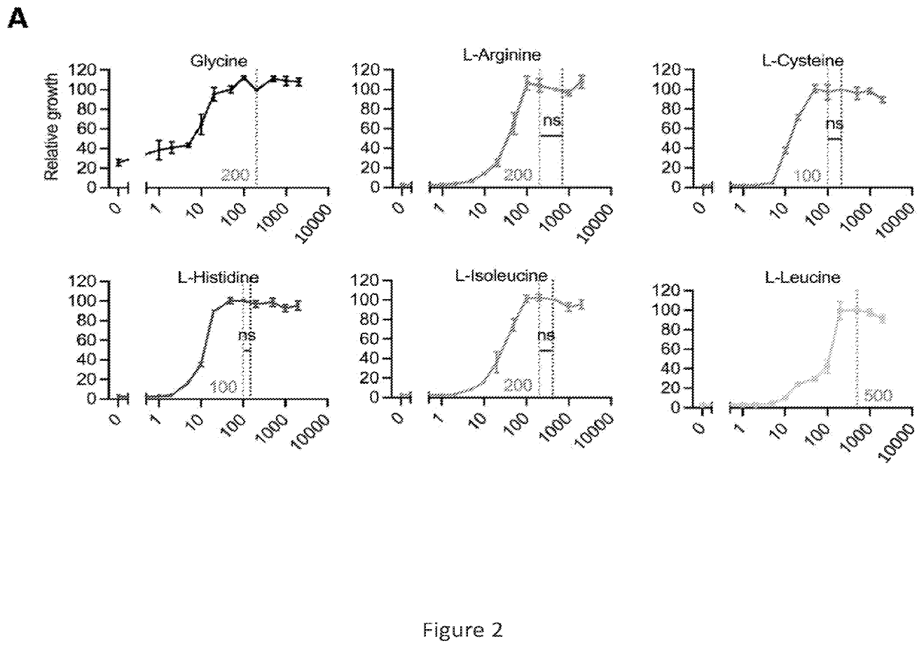

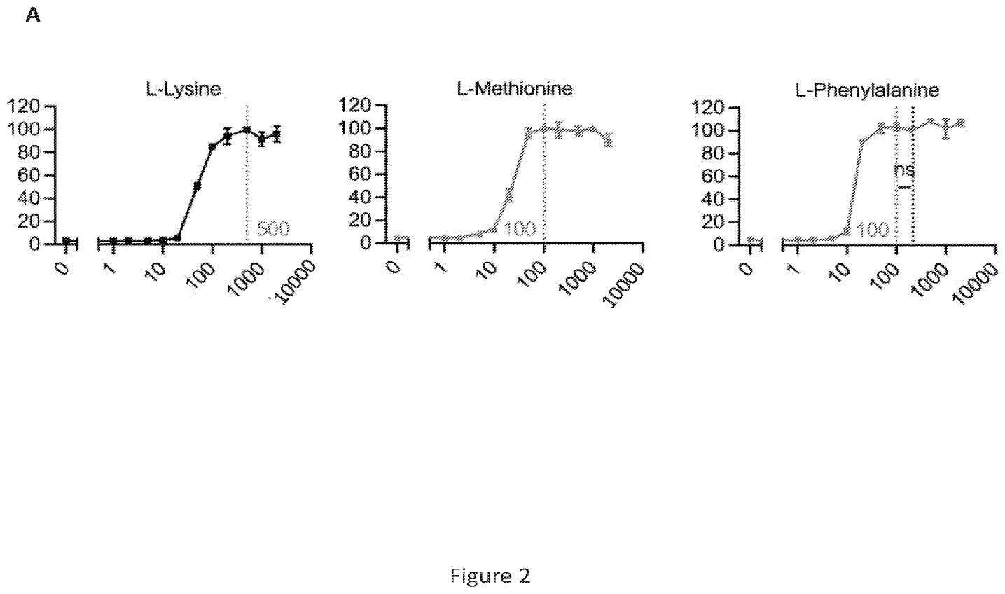

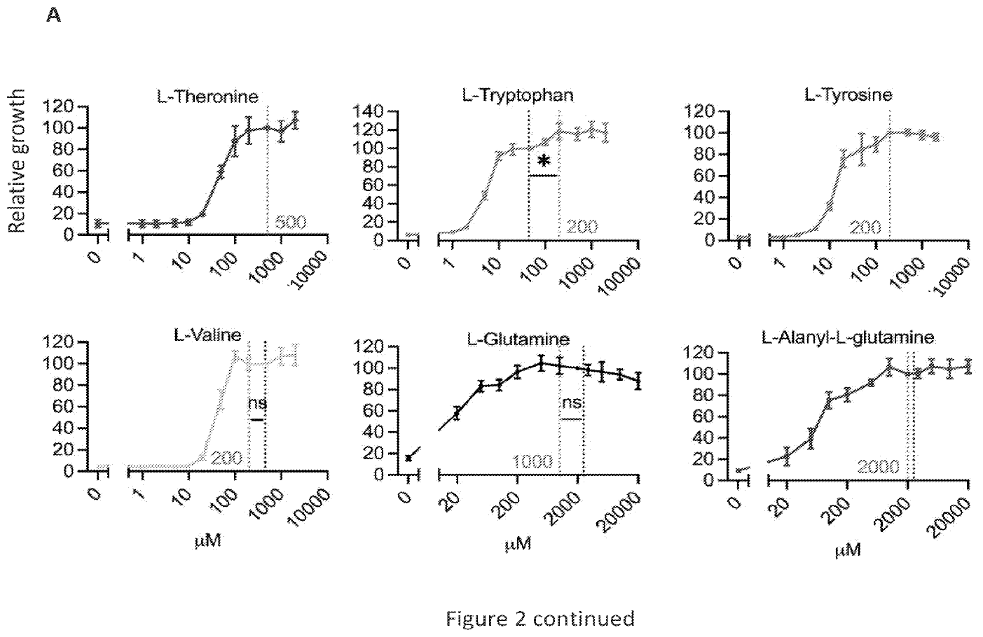

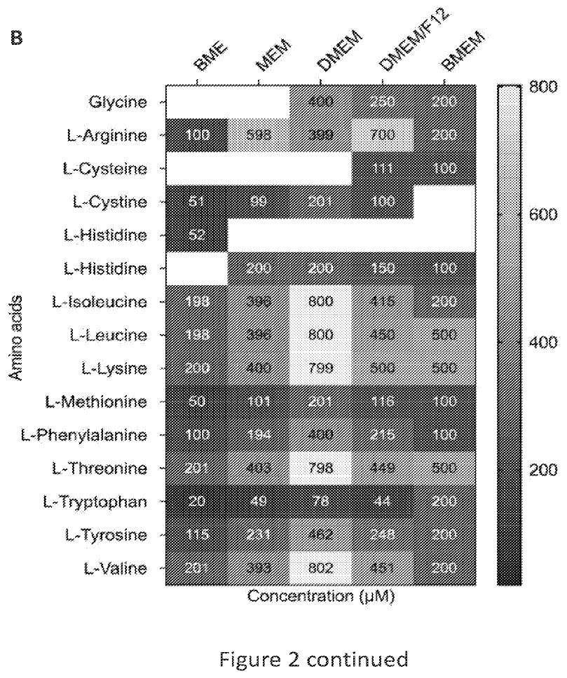

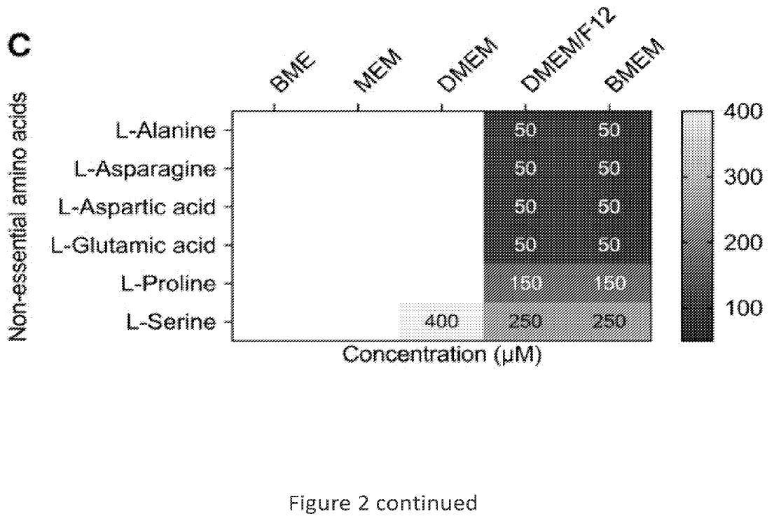

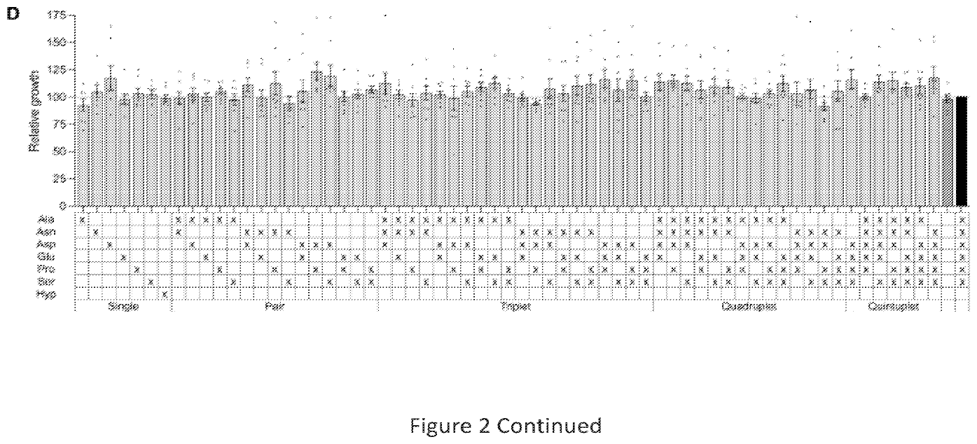

The necessity and suitable concentrations of amino acids were investigated, again using a two-passage assay. The amino acids were separated into two groups based on their previously determined essentiality in vitro: essential amino acids (EAAs) glycine, arginine, cystine/cysteine, histidine, isoleucine, leucine, lysine, methionine, phenylalanine, threonine, tryptophan, tyrosine, valine, and glutamine; and NEAAs, alanine, asparagine, aspartic acid, glutamic acid, proline, and serine. An initial evaluation showed that cysteine alone can replace the cystine and cysteine combination found in DMEM/F12; therefore cystine, which has poor solubility, was eliminated. From concentration titration assessments, all EAAs were proven essential in the daily medium change experiments (FIG. 2A). Interestingly, in the no medium change experiments, addition of glycine was superfluous, suggesting that hiPSCs can synthesize this glycine but at an inadequate rate (FIG. 3C). Seven of the EAAs, including glutamine, were suitable at lower concentrations than those in DMEM/F12 (FIGS. 2B, 3D, and 3E), although none of the DMEM/F12 concentrations appear to have a negative effect on growth (FIG. 2A). Conversely, the suitable concentration of tryptophan was found to be ˜4-fold higher than that in DMEM/F12. Finally, DMEM/F12 concentrations of histidine, leucine, lysine, methionine, threonine, and tyrosine were shown to be approximately suitable. Whether the replacement of glutamine, a potential secondary source of energy in vitro, with the more stable L-alanyl-L-glutamine (GlutaMAX) was feasible was evaluated. However, 2 mM L-alanyl-L-glutamine was required to maintain similar levels of proliferation to those of 1 mM glutamine, which would have a significant cost impact in the final formulation. Comparing these suitable concentrations, no difference in hiPSC growth rate was observed, even when using a no medium change strategy (FIGS. 3C-3E).

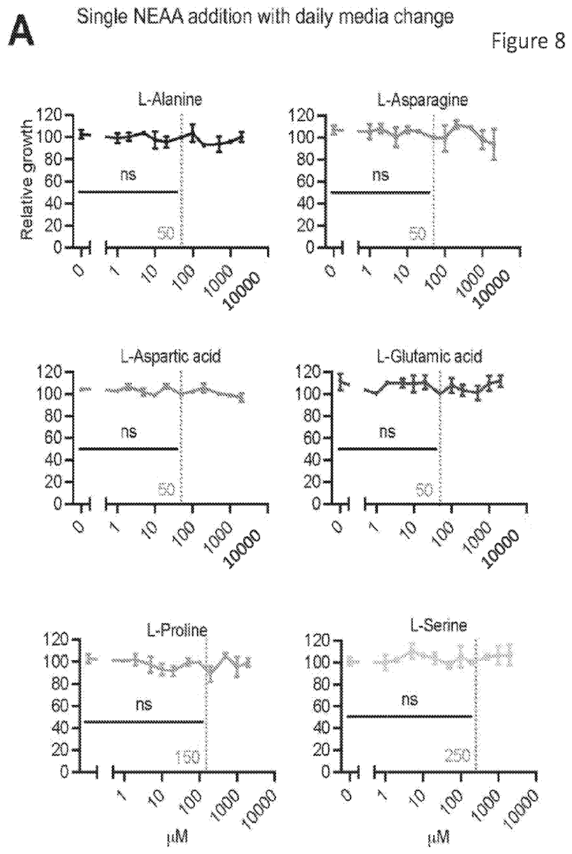

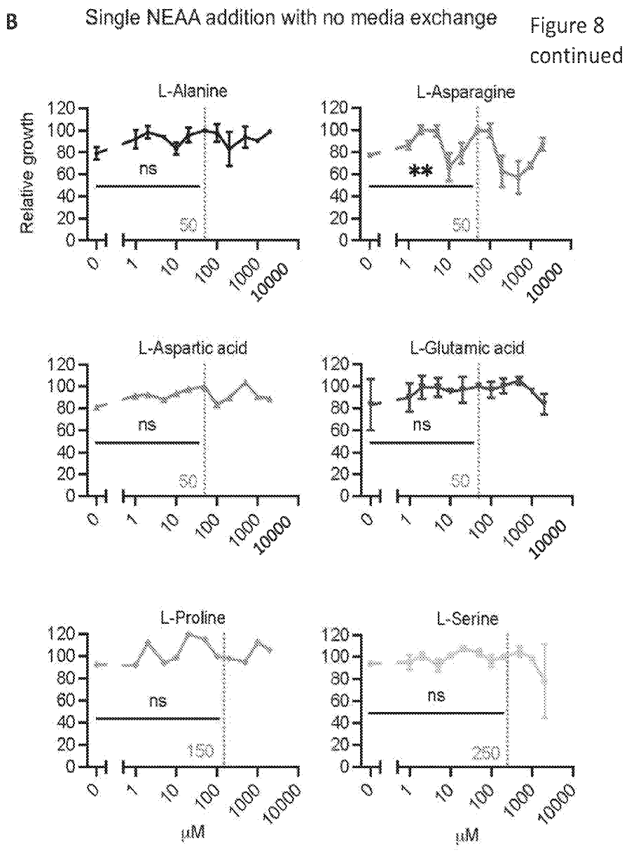

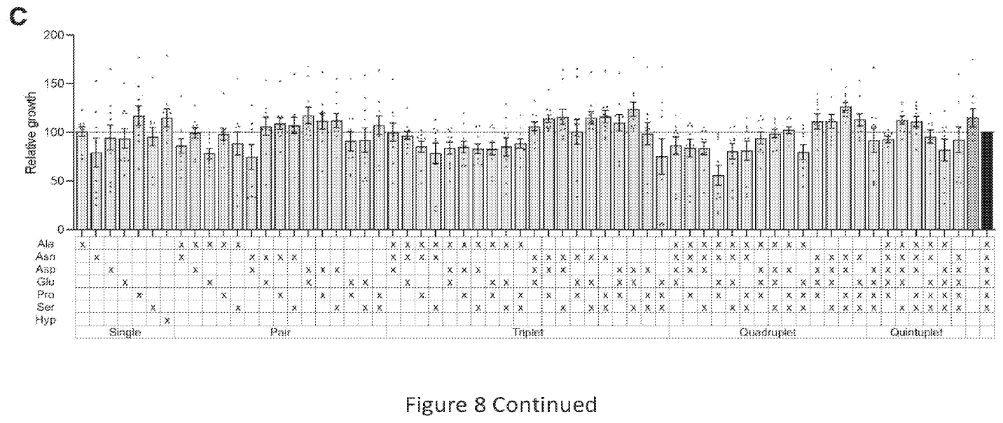

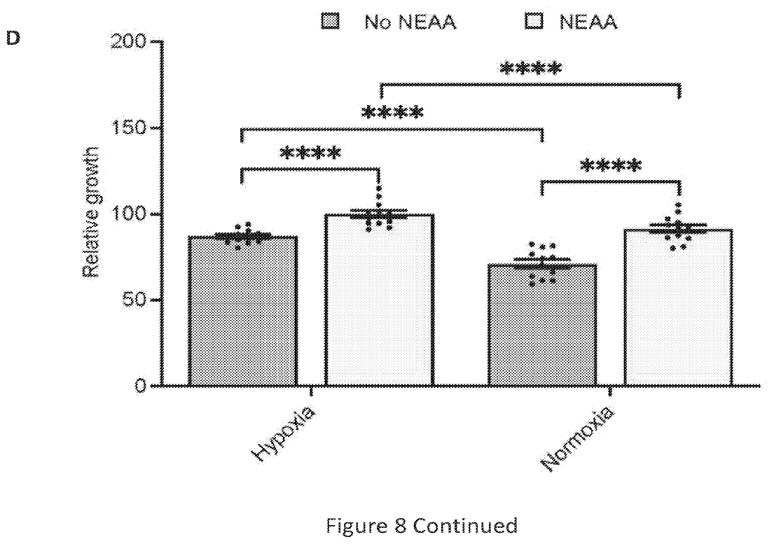

The necessity of NEAAs in medium formulae is well-known to be difficult to establish, and information on where the currently used concentrations of NEAAs were derived from is not described (Lockart and Eagle, 1959). To assess the necessity of NEAAs included in the DMEM/F12 formula (FIG. 2C), the same two-passage assay completed for the EAAs was initially performed, but no effect of the absence of any one individual NEAA was found (FIGS. 8A and 8B). This finding suggests that NEAAs may have some level of redundancy and/or require longer to deplete, and therefore led us to assess the effect of the addition of NEAAs individually, in pairs, triplets, quadruplets, or quintuplets, in a longer five-passage assay at the concentrations found in DMEM/F12. In this longer assay, the absence of all six NEAAs had no significant negative effect on growth and even the most successful asparagine/proline combination was not significantly better than no NEAAs (FIG. 2D). Similar results were seen with the no medium change strategy, with no clear superior combination (FIG. 8C) Upon further thorough testing the role of NEAAs with a daily medium change strategy, BMEM versions without NEAA was found to exacerbate the negative effect of normoxic conditions (21% O₂) on growth over our typical hypoxic (5% O₂) conditions (FIG. 8D), and further validated the need of NEAAs with a daily medium change schedule. Therefore, although NEAAs are clearly not essential to the BMEM formula, the NEAAs were kept for medium robustness under a variety of conditions, and only cystine was eliminated.

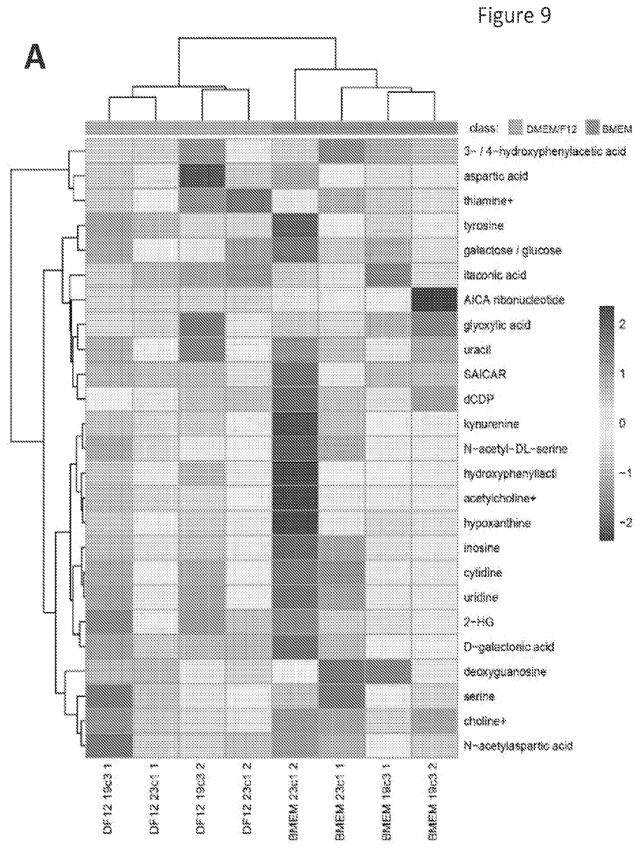

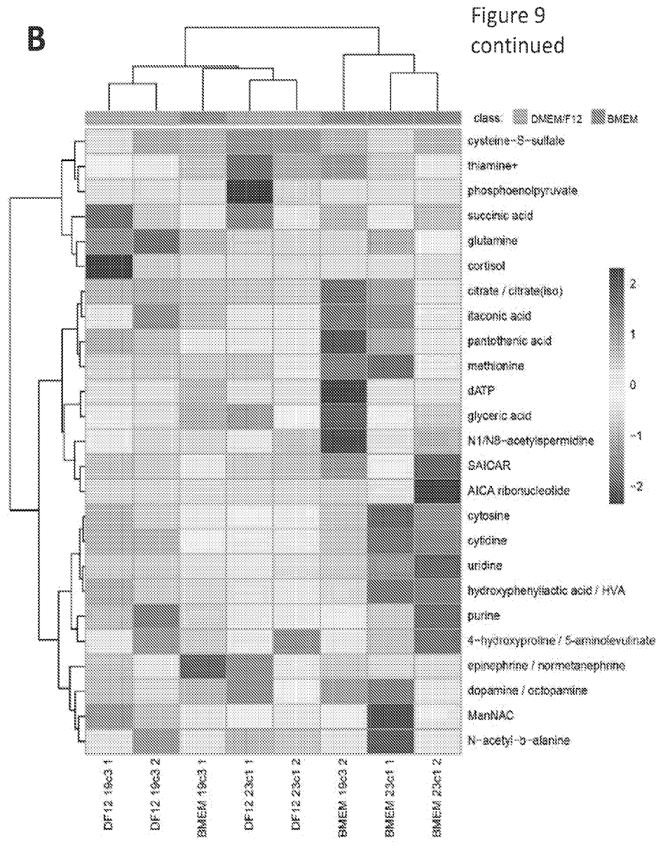

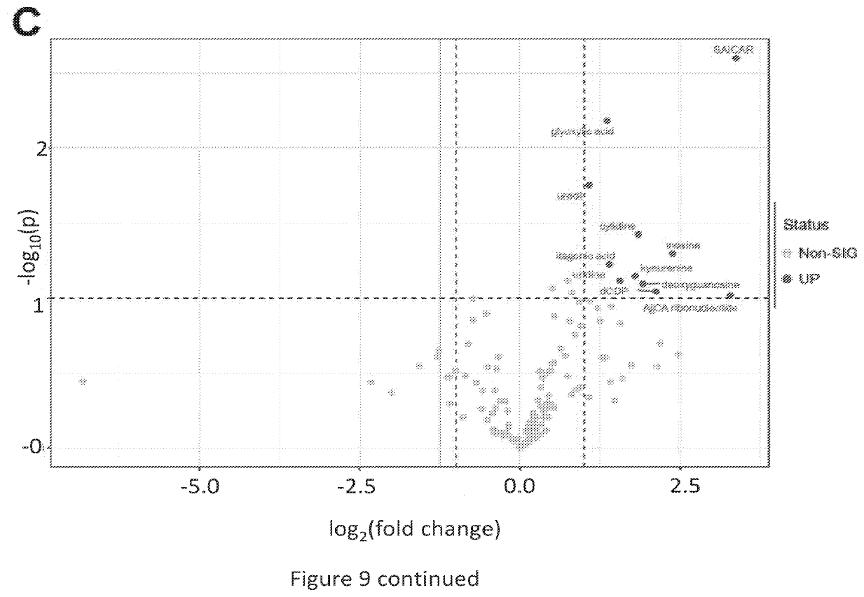

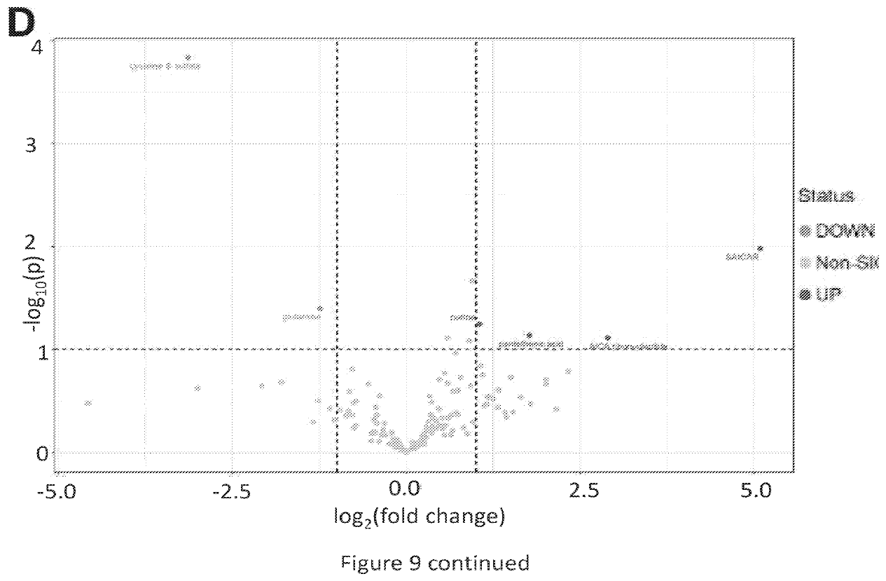

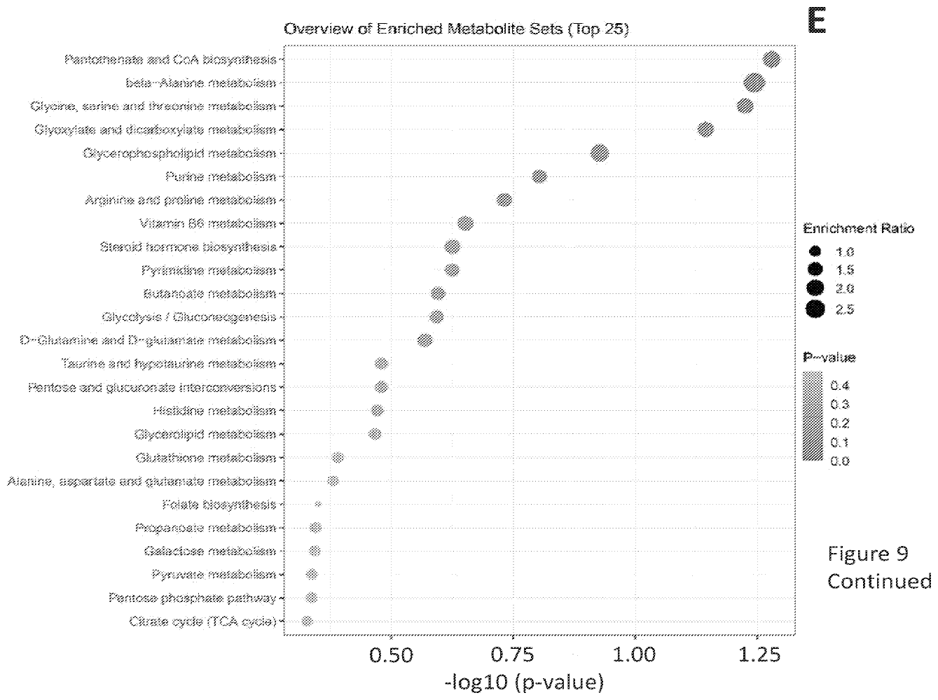

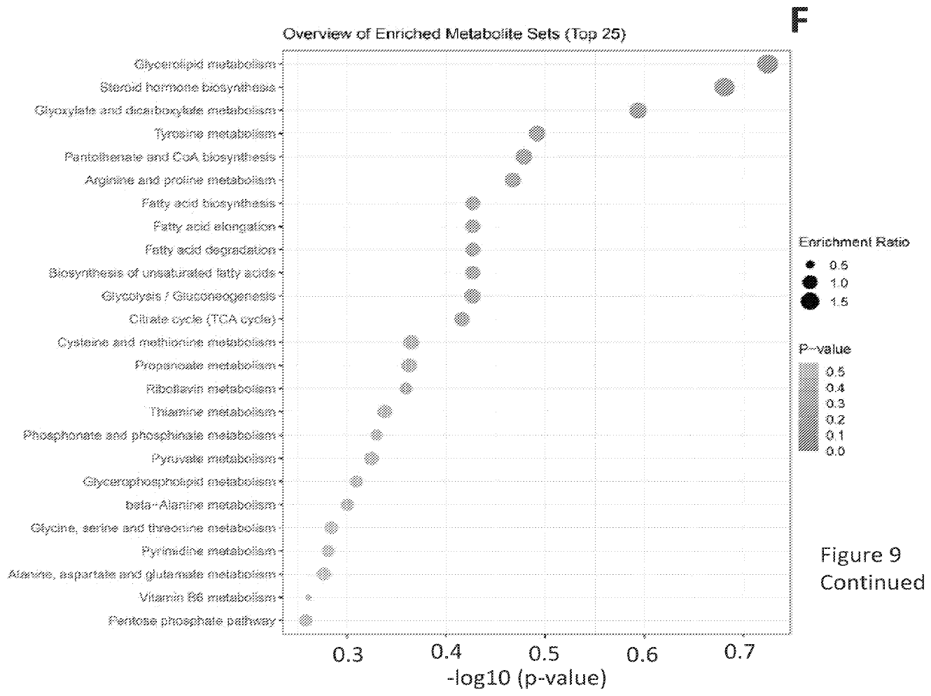

Intracellular metabolite analysis found very little difference between cells cultured in BMEM vs. DMEM/F12 with either a daily or no change strategy (FIG. 9A, 9B, 10A, 9B). Phosphoribosylaminoimidazolesuccinocarboxamide (SAICAR), an intermediate in the formation of purines, was the sole metabolite equally upregulated in BMEM cells with and without daily medium change (FIG. 9C-F).

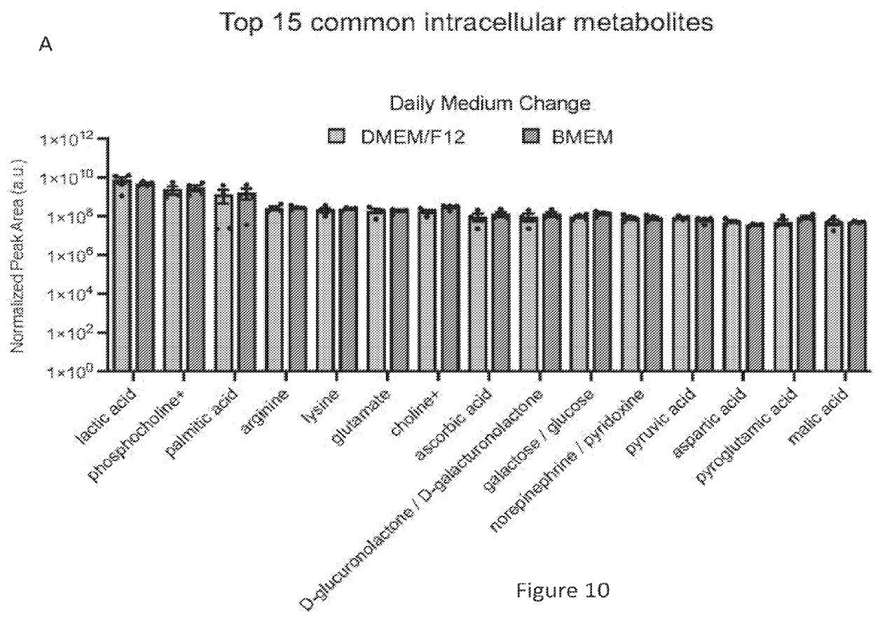

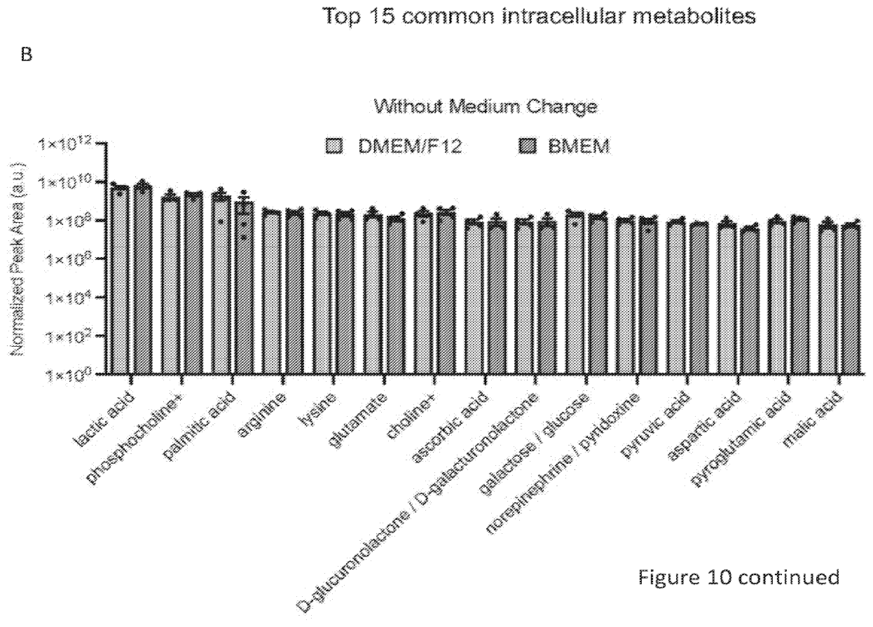

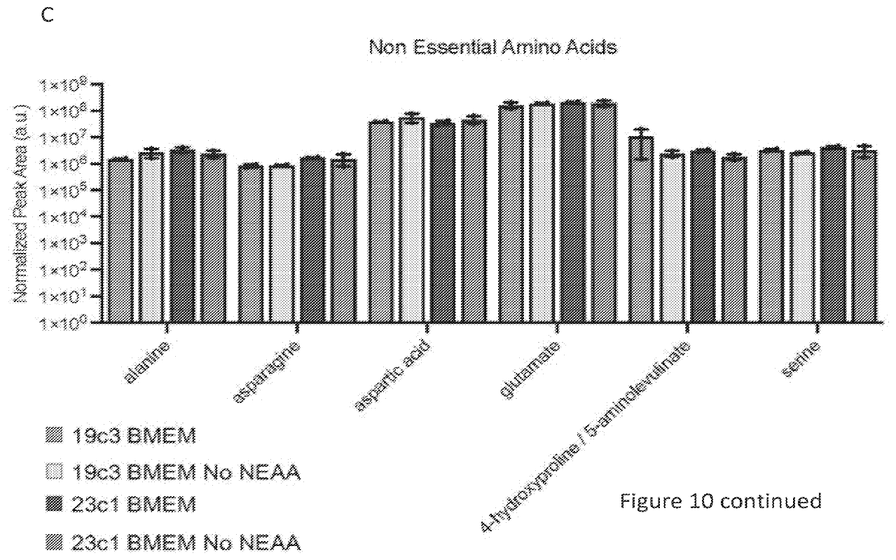

A version of BMEM was also formulated without the six NEAAs and quantified their intracellular content to investigate the hypothesis of their de novo synthesis by hiPSCs. Of the six NEAAs, only proline was not detected in hiPSCs, although 4-hydroxyproline was abundant. In addition, the intracellular concentrations of all NEAAs was found to be broadly similar (FIG. 10C), suggesting that hiPSCs can synthesize these compounds as needed, after adaptation to culture conditions where they are absent.

Finally, metabolomics analysis measured two “other” components, hypoxanthine and thymidine, that are present in DMEM/F12 but not BMEM. Hypoxanthine was at a higher concentration in BMEM cultured cells, suggesting that these cells can generate this by deamination of adenine. Thymidine was also measured but not found to be present in either DMEM/F12 or BMEM cultured cells. These results further validate the removal of both components in our formulation of BMEM

### Vitamins

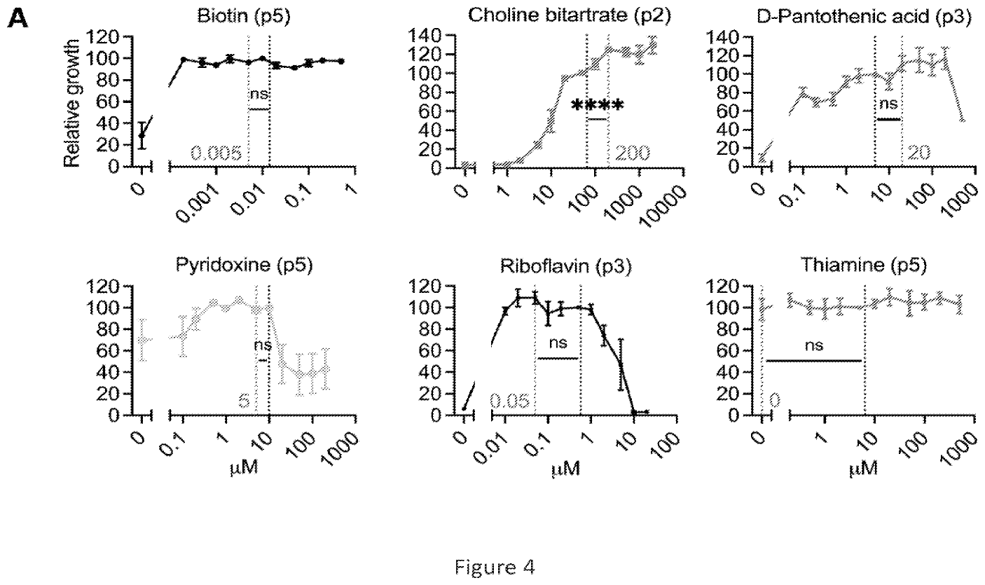

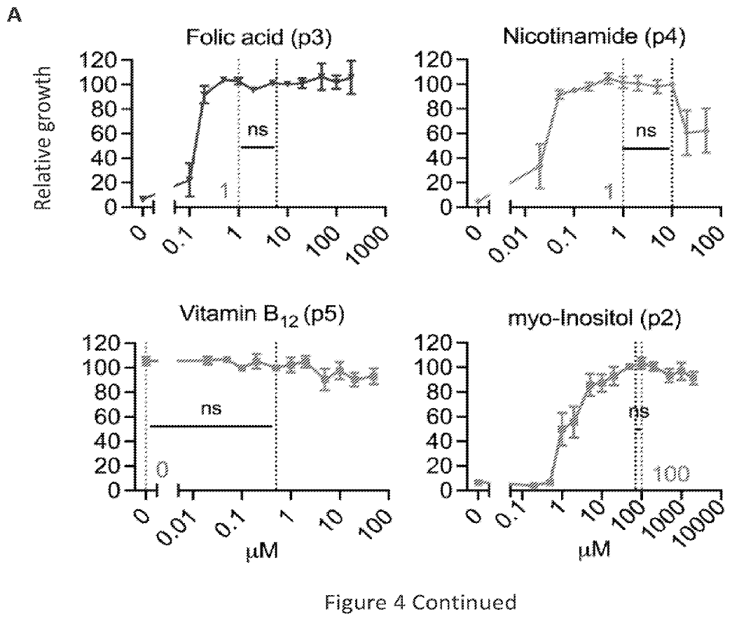

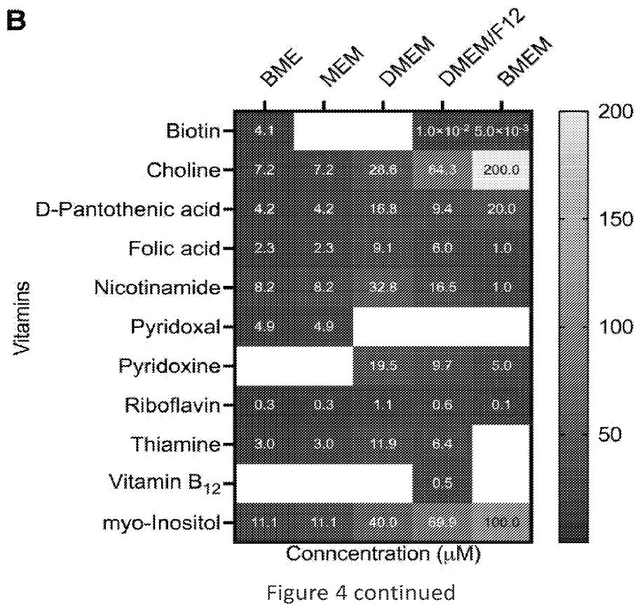

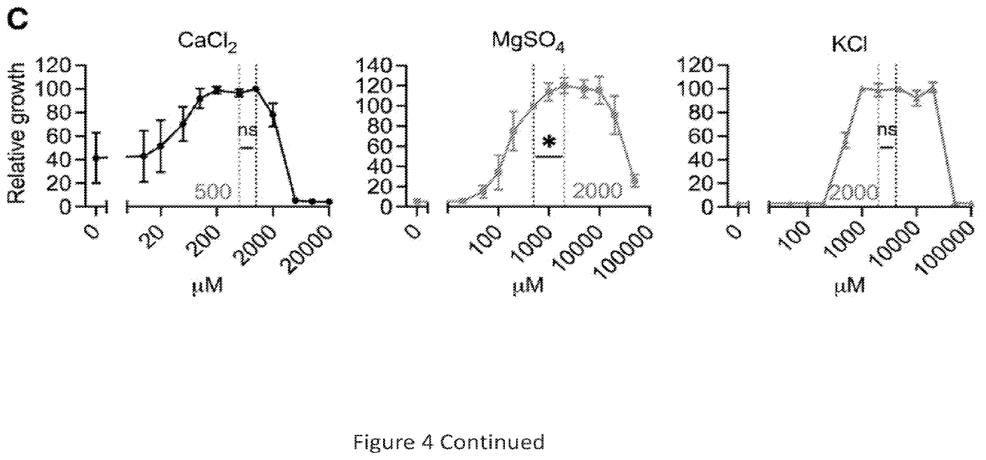

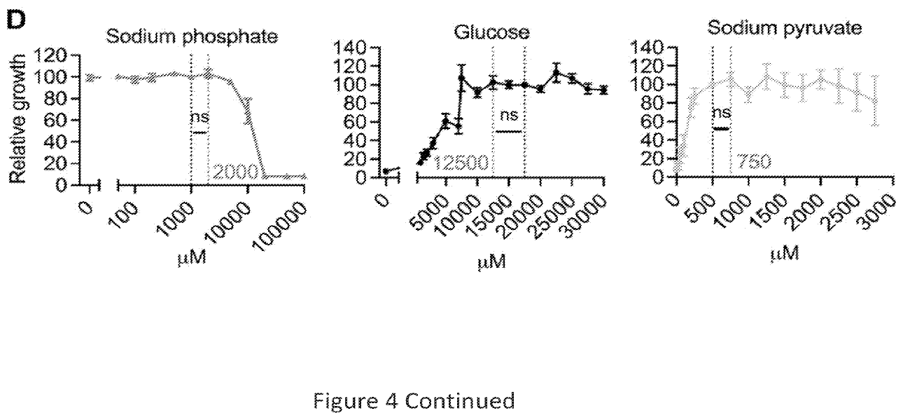

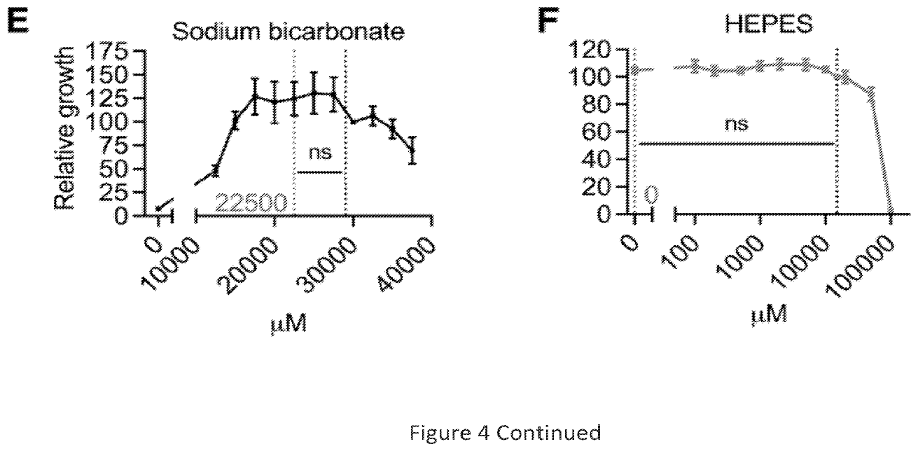

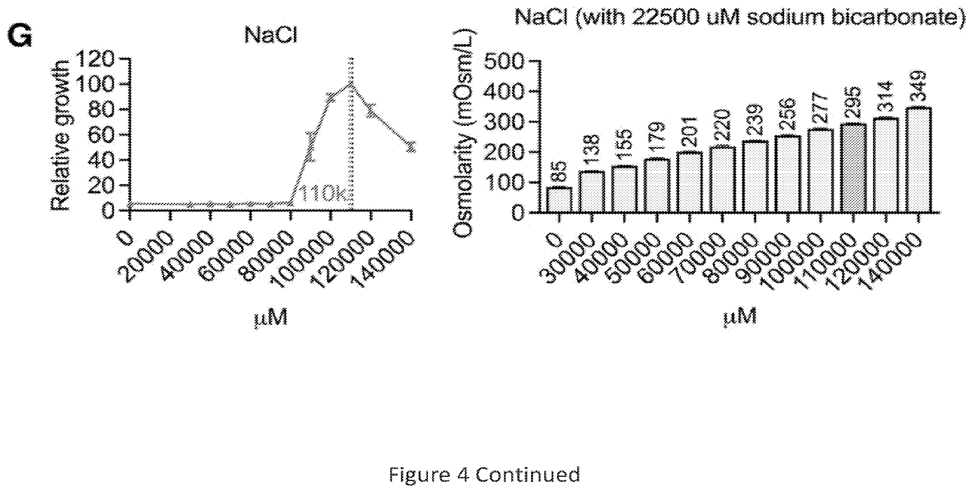

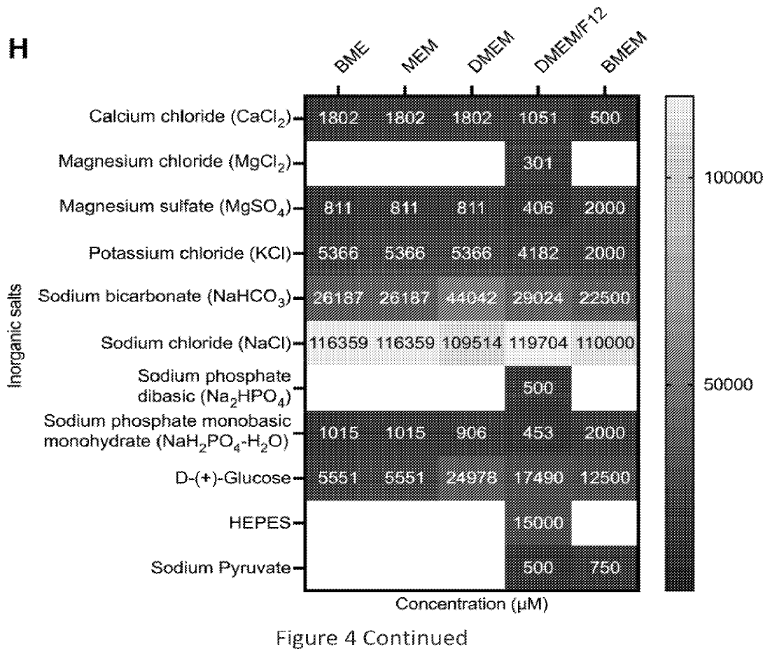

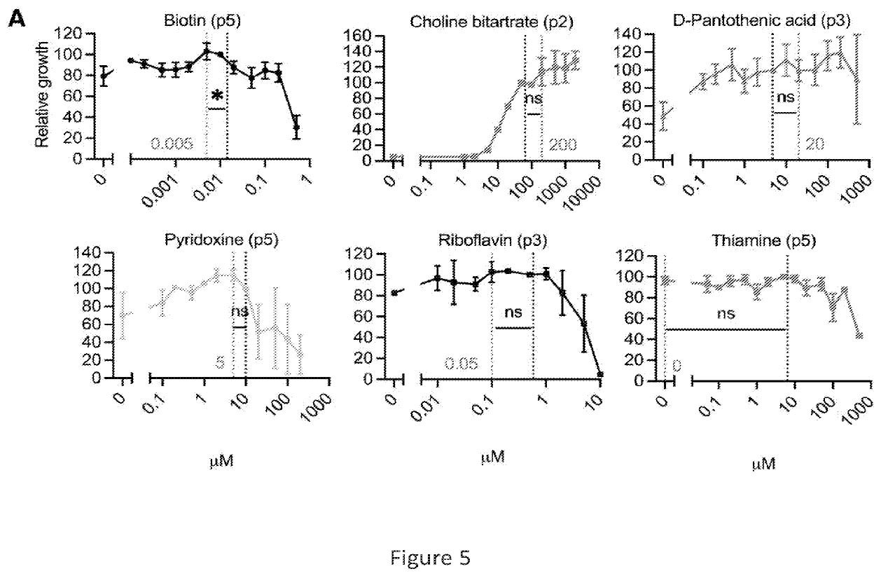

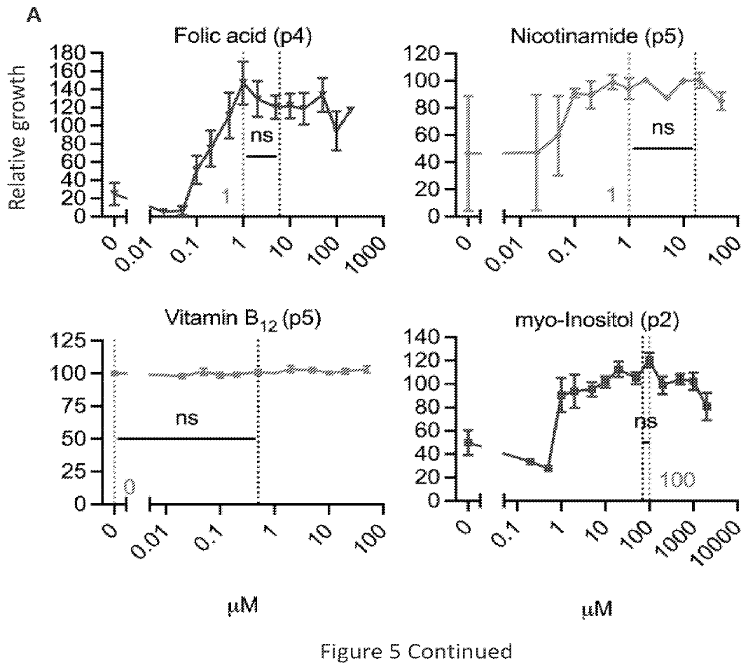

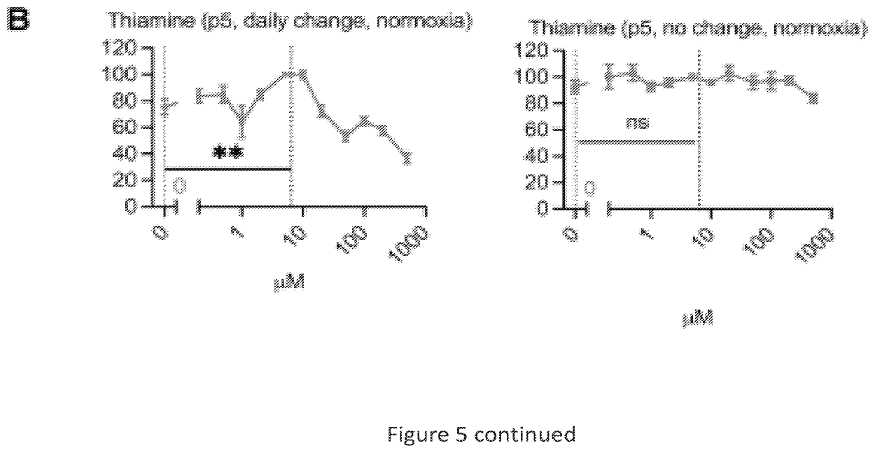

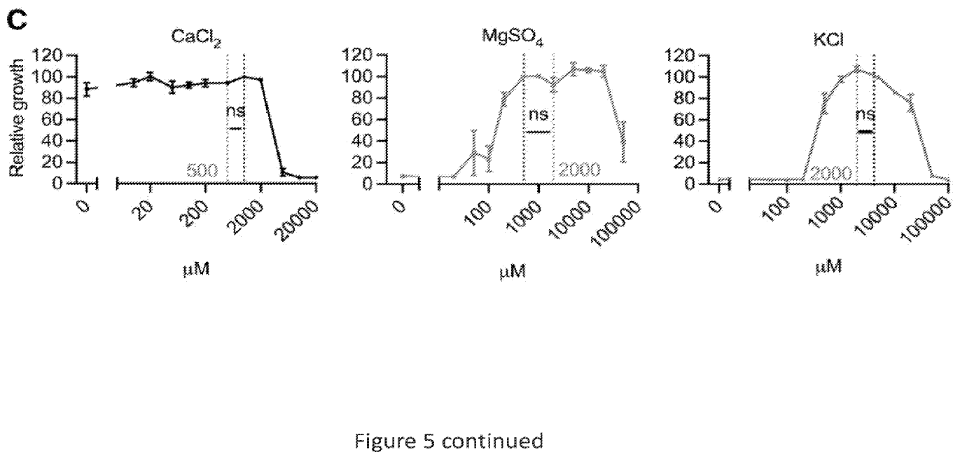

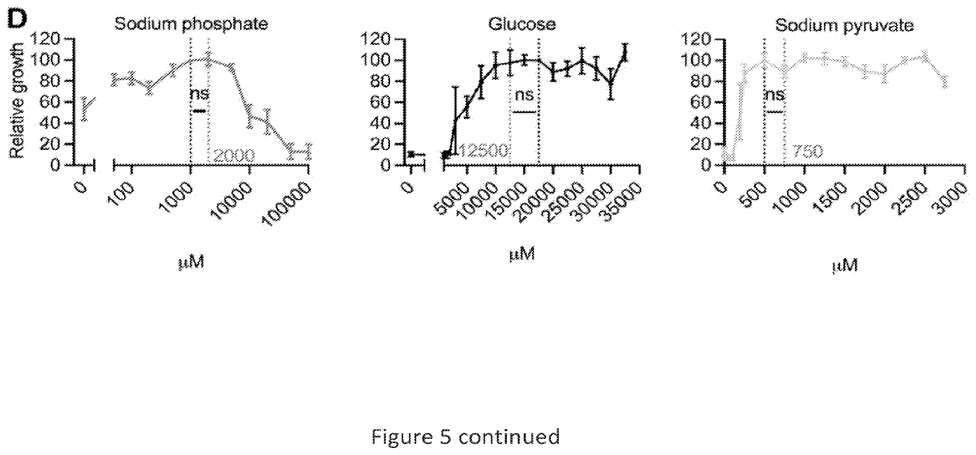

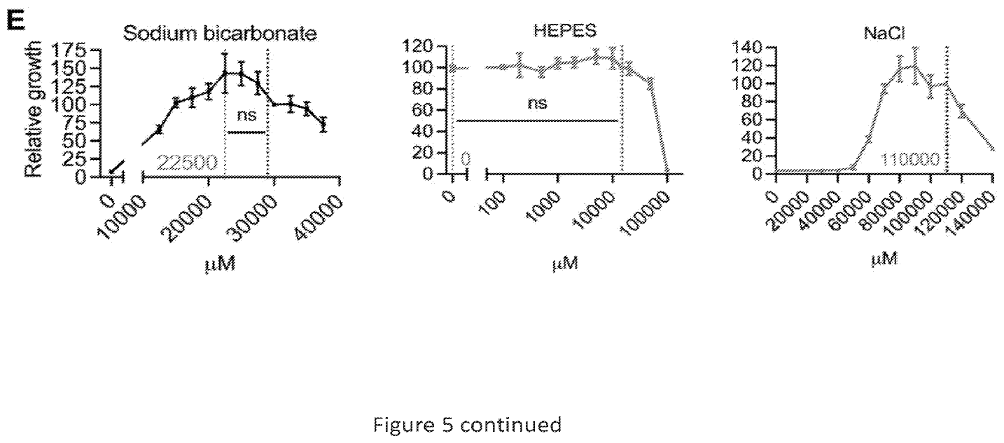

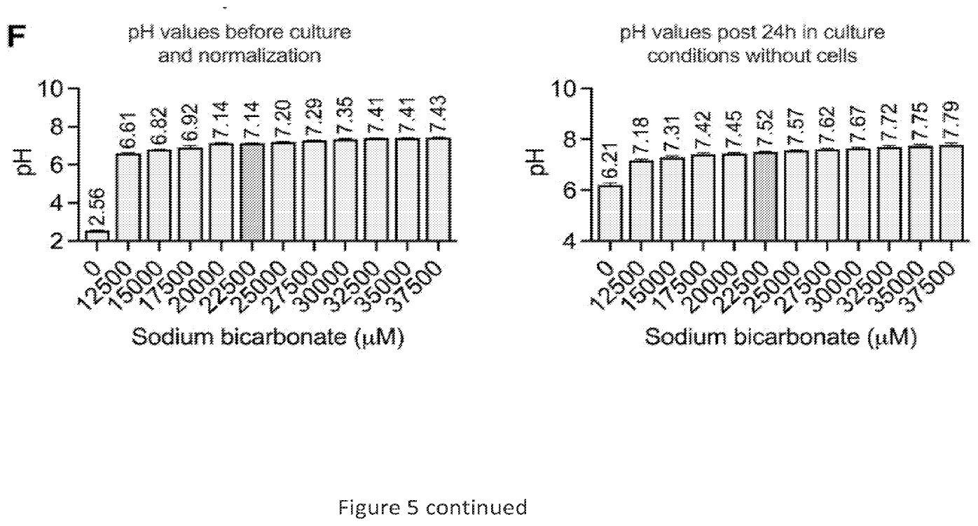

To titrate vitamin concentrations, the first step was the replacement of choline chloride with choline bitartrate to avoid the hygroscopic clumping of choline chloride and improve the solubility. The following experiments demonstrated that biotin, folic acid, nicotinamide, pyridoxine, and riboflavin were all sufficient at concentrations lower than those in DMEM/F12, whereas higher concentrations of choline, D-pantothenic acid, and myoinositol were beneficial (FIG. 4A). Similar results were seen in the no medium change experiments (FIG. 5A). The minimum number of passages required to identify the effect of deprivation of each vitamin was found to vary from two to five passages. We found that both thiamine and Biz are superfluous for hiPSC growth (FIG. 4A), although thiamine was required under normoxic conditions (FIG. 5B). Thiamine is a coenzyme involved in pyruvate metabolism, the pentose phosphate pathway, and the citric acid cycle, and is included in all common basal media formulae other than BMEM (FIG. 4B). B12 is a coenzyme in fatty acid metabolism but is not included in the BME, MEM, or DMEM formulae.

### Inorganic Salts

The suitable concentrations of the inorganic salts of calcium, magnesium, and potassium was studied. DMEM/F12 contains both MgCl₂ and MgSO₄, while other basal medium formulae typically use MgSO₄ alone, so we began by eliminating MgCl₂. Individual titration experiments and found that a two-passage assay was suitable and that, for the CaCl₂ and KCl ions, the existing concentrations in DMEM/F12 could be reduced (FIG. 4C). Of note, rather than low viability, low calcium concentrations reduced the adhesion of hiPSCs to the plates, whereas high concentrations were not soluble. We also found that an increase in MgSO₄ concentration enhanced growth (FIG. 4C). Similar results were seen in the no medium change experiments (FIG. 5C).

Sodium phosphate, glucose, and sodium pyruvate DMEM/F12 contains two types of sodium phosphate so we began by eliminating the dibasic version, which has poor solubility. We found that, when the medium was suitably pH adjusted, sodium phosphate monobasic was superfluous in the daily medium change assay (FIG. 4D), whereas it was essential at around 2 mM in the no medium change assay (FIG. 5D), likely due to its role in maintaining pH. The effect of glucose appeared relatively stable from 10 to 30 mM; therefore, 12.5 mM concentration was conservatively chosen (FIG. 4D). Sodium pyruvate was shown to be essential and most efficacious at a slightly higher concentration than found in DMEM/F12. Of note, pyruvate, although predominantly known as a source of acetyl-CoA for the citric acid cycle, also has a role in cell culture as an antioxidant (Ramos-Ibeas et al., 2017).

### Sodium Bicarbonate, HEPES, and pH

All common basal media use a bicarbonate buffering system allowing for the balance of CO₂ in cell culture incubators (typically 5%) to maintain the pH of the cultures. Our previous work has shown that pH 7.1 is most suitable for hiPSC growth (Kuo et al., 2020). We found that, by normalizing the pH to 7.1 as we varied sodium bicarbonate concentrations, a lower level than was suggested previously (Chen et al., 2011) (22.5 mM) was most suitable for hiPSC growth (FIGS. 4E, 5E, and 5F). This concentration is even lower than is typically found in other basal media (FIG. 4H) We also found that HEPES, a second buffering agent, is superfluous (FIG. 3F), even in a no medium change method (FIG. 5E). As HEPES is responsible for greater than half of the cost of BMEM, this is a useful component to be excluded. The elimination of HEPES from the basal medium allowed us to reduce our formula to 39 components. Of note, the primary role of HEPES is to maintain the pH of the medium when the plate is outside of CO₂ incubator. We found that during typical culture HEPES was not essential but if plates are going to be more than briefly observed under the microscope then the pH will quickly become basic and HEPES should be included.

### NaCl and Osmolarity

Maintenance of physiological osmolarity is essential for cell culture and NaCl makes up greater than 50% of the mass of typical powdered media. An osmolarity of 310 mOsm/L was previously found to be suitable (Kuo et al., 2020), which was broadly similar to 330 mOsm/L found by others (Ludwig et al., 2006). Here, 110 mM of NaCl is suitable, which produced a slightly lower osmolarity of 295 mOsm/L (FIG. 4G).

### Effect of Basal Medium on Gene Expression

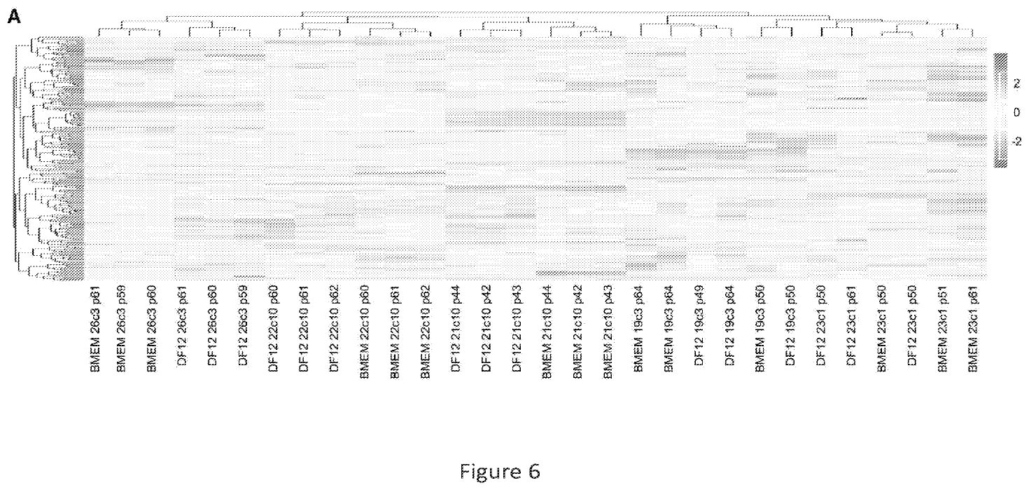

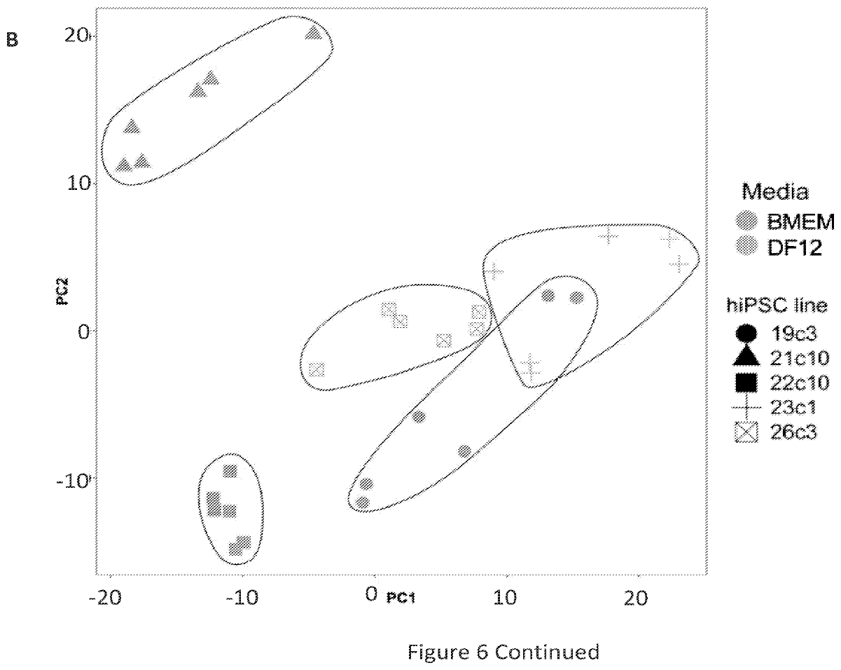

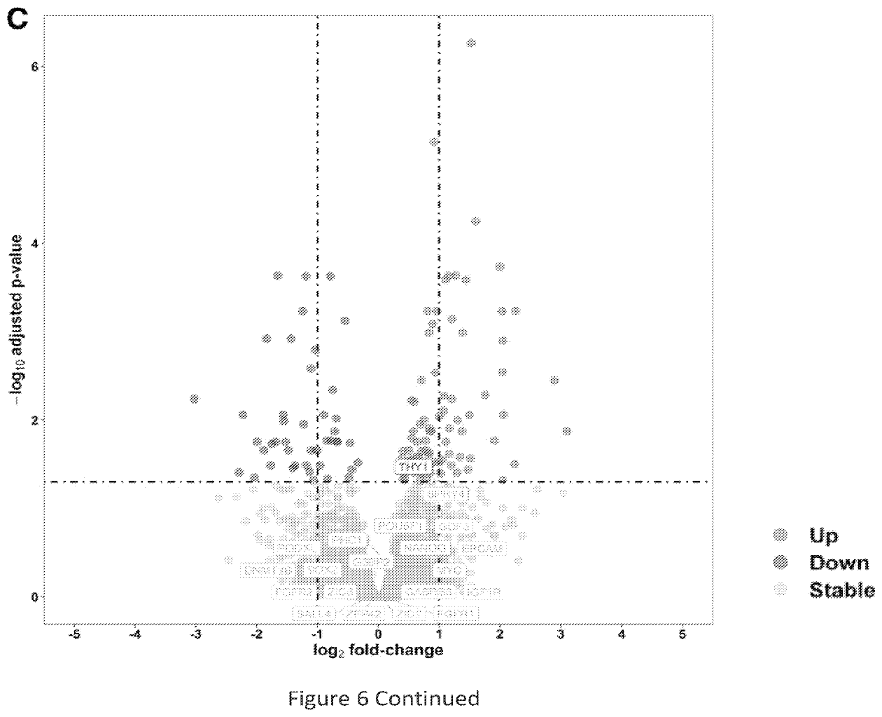

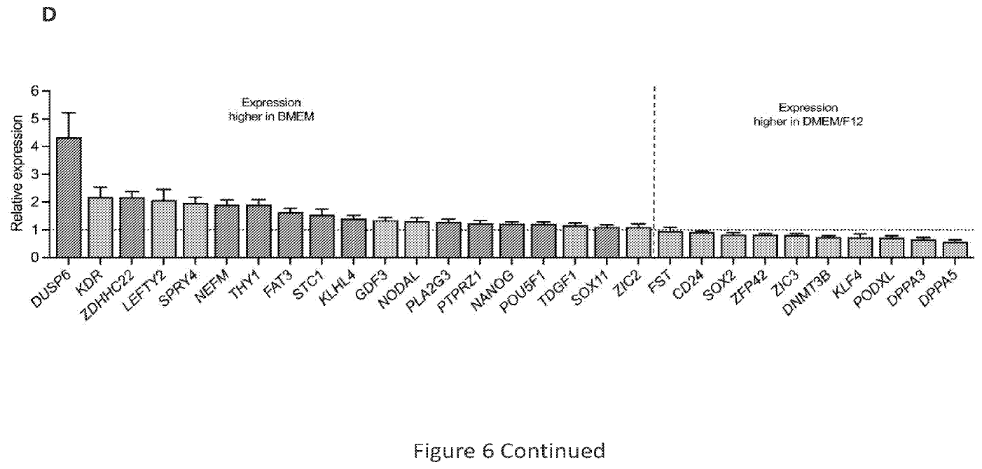

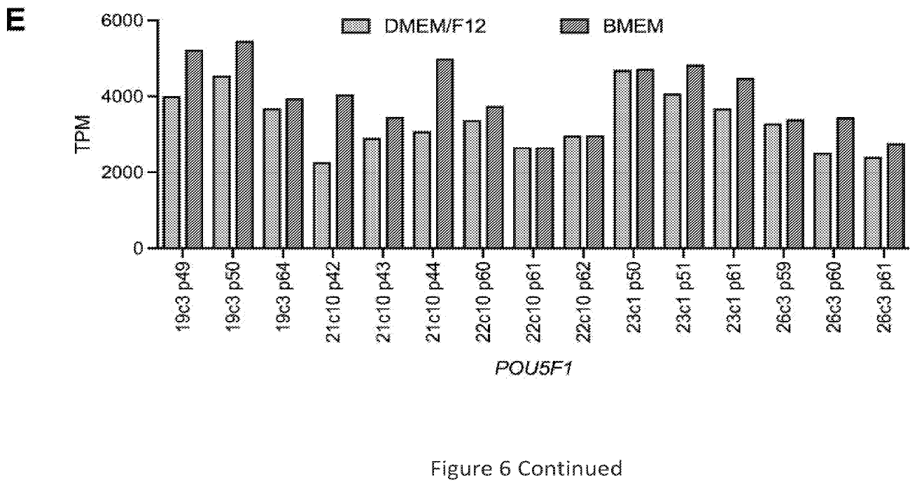

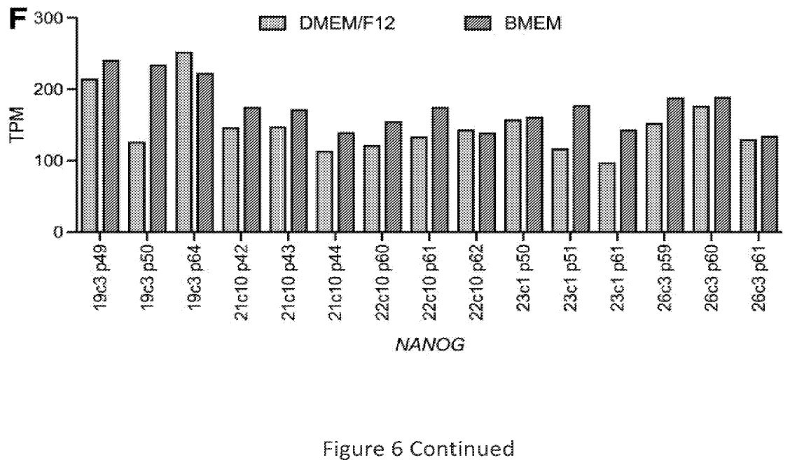

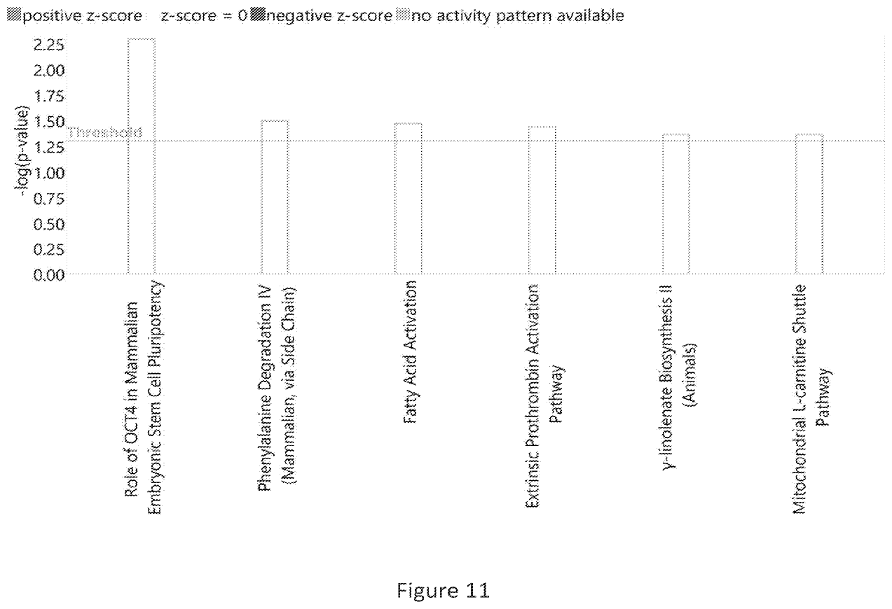

RNA sequencing (RNA-seq) was completed on 15 paired samples cultured either in BMEM or DMEM/F12. Hierarchical clustering of these samples based on the top 250 most variable genes demonstrated that all samples clustered based on the cell line rather than the medium in which they were cultured (FIG. 6A). A principal-component analysis of the RNA-seq data further confirmed this observation (FIG. 6B) Numerous undifferentiated cell genes were found to be expressed significantly higher in hiPSCs cultured in BMEM compared with DMEM/F12, including KDR, SPRY4, GDF3, NODAL, NANOG, POU5F1, TDGF1, and ZIC2 (FIGS. 6C and 6D). Conversely, PODXL, DMNT3B, ZFP42, and SOX2, all had lower expression in BMEM. Interestingly, we found markers of the primed state to be most upregulated in BMEM cultured cells such as DUSP6, ZDHHC22, NEFM, THY1, FAT3, STC1, KLHL4, PLA2G3, PTPRZ1, and SOX11 (Messmer et al., 2019). Conversely, genes associated with the naive state such as FST, CD24, ZIC3, DPPA3, and DPPA5 had lower relative expression in BMEM Studying individual genes such as POU5F1 and NANOG (FIGS. 6E and 6F), the upregulation in BMEM is shown to be conserved across most of our samples. Using QIAGEN Ingenuity Pathway Analysis, the only pathway significantly upregulated in cells cultured in BMEM was “role of OCT4 in mammalian embryonic stem cell pluripotency” (FIG. 11).

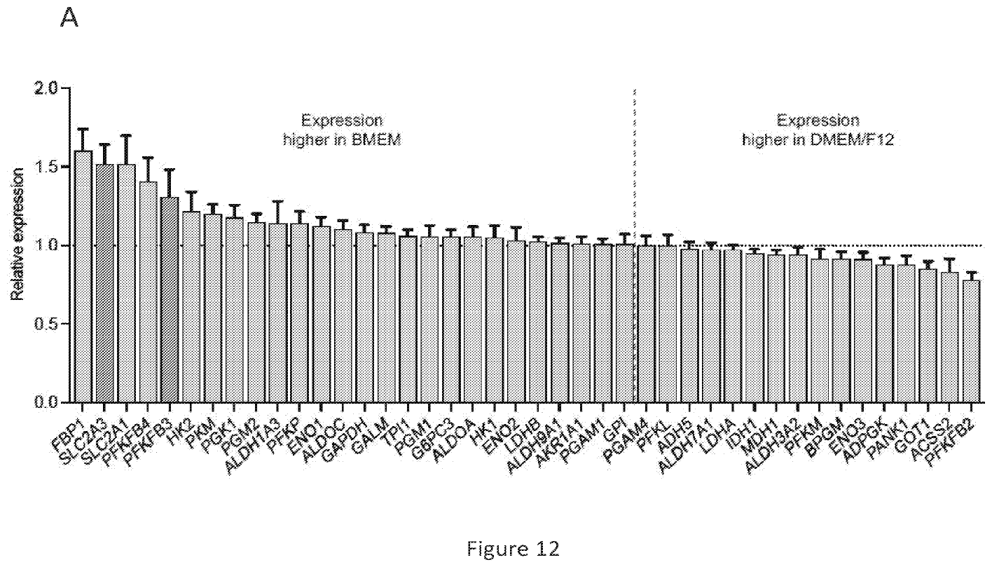

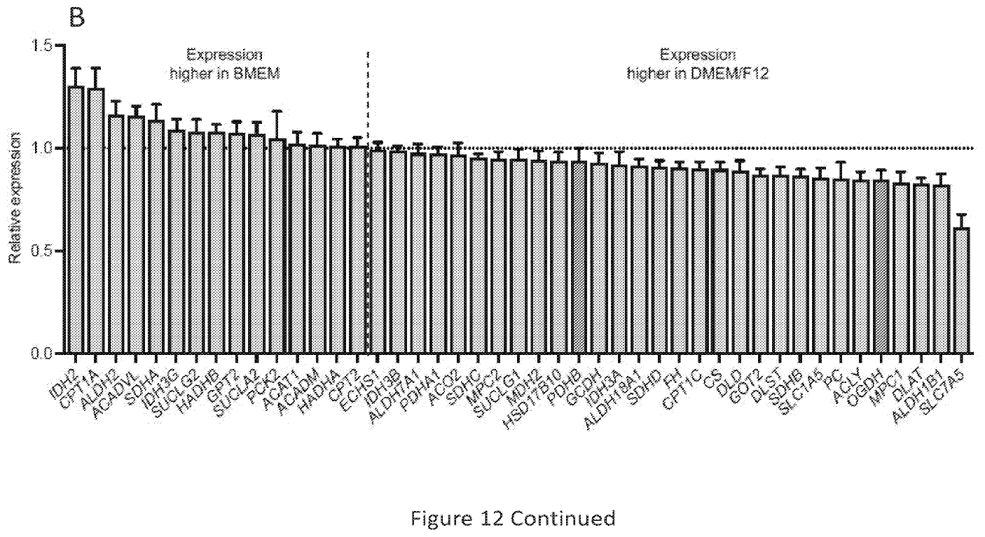

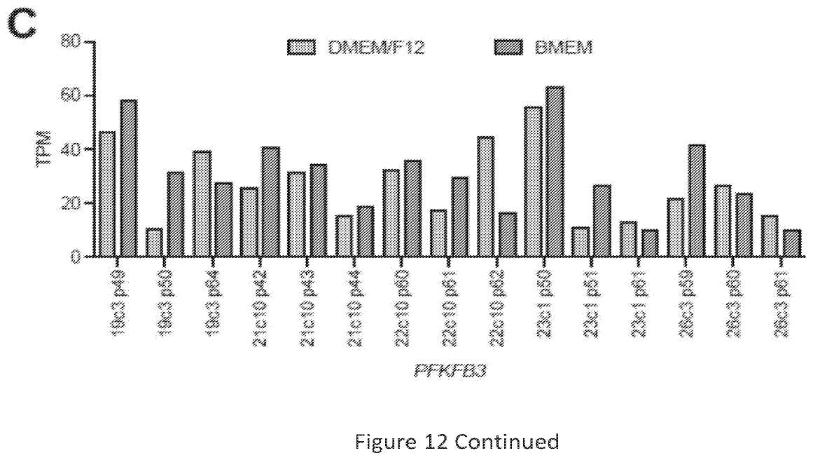

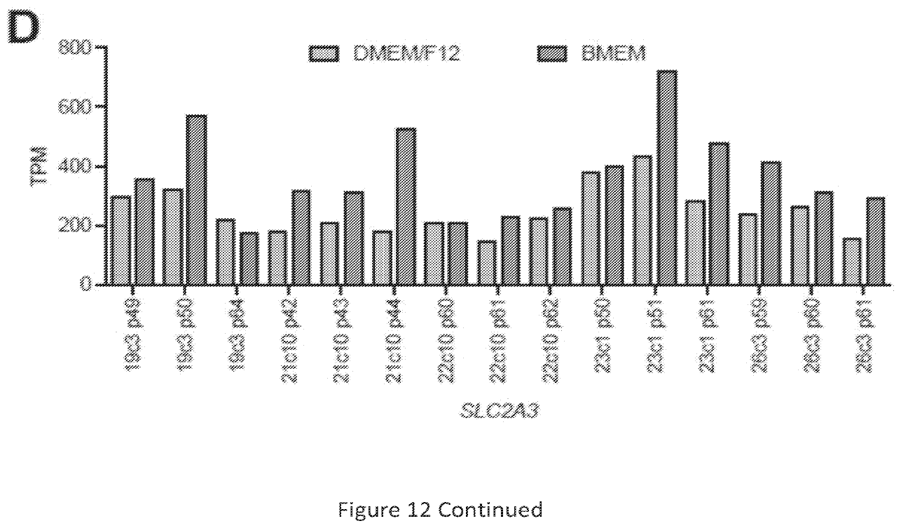

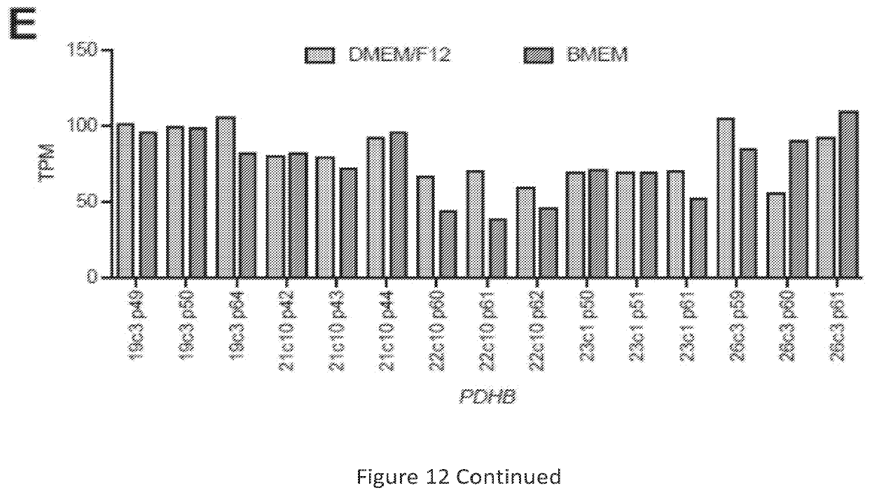

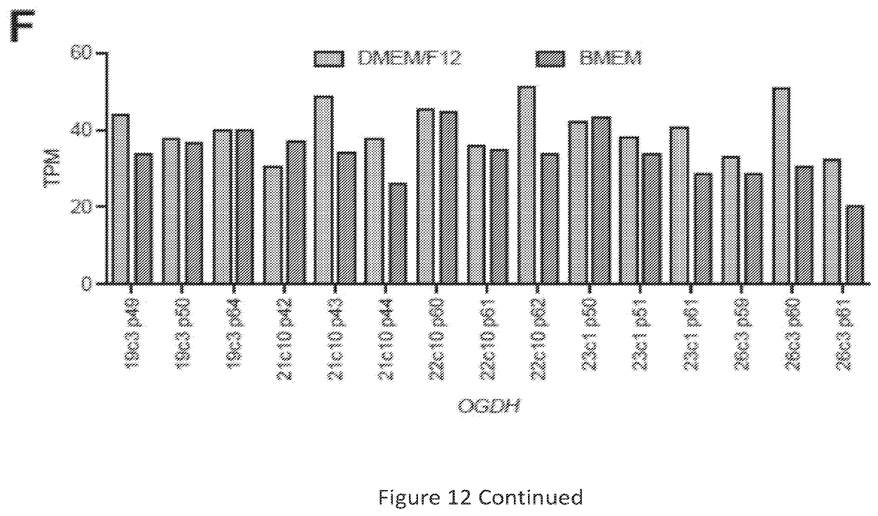

Knowing that many of the medium components that were removed are involved in cellular respiration, genes associated with metabolism were specifically studied. In BMEM, numerous genes associated with glycolysis were observed to be upregulated, such as ENO1, HK1, HK2, PGK1, GAPDH, and PFKFB3 (FIGS. 12A and 12C), and particularly glucose transporter proteins 1 and 2 (SLC2A1 and SLC2A3) (FIG. 12D) Conversely, mitochondrial metabolism-related genes were downregulated in BMEM-cultured cells, these include components of the pyruvate dehydrogenase complex, DLAT, PDHB, SDHB, SDHC, and SDHD (FIGS. 12B and E), components of the oxoglutarate dehydrogenase complex including OGDH (FIG. 12F), and DLST, consistent with cases of thiamine deficiency (Bubber et al., 2004).

Characterization and Differentiation of BMEM-Cultured hiPSC Lines

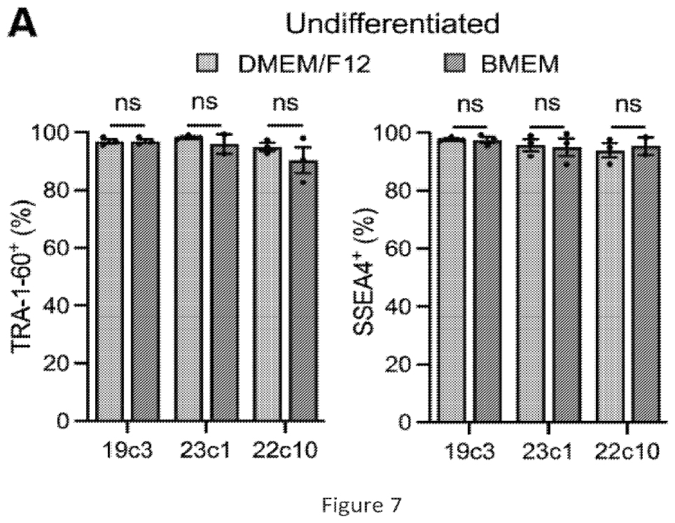

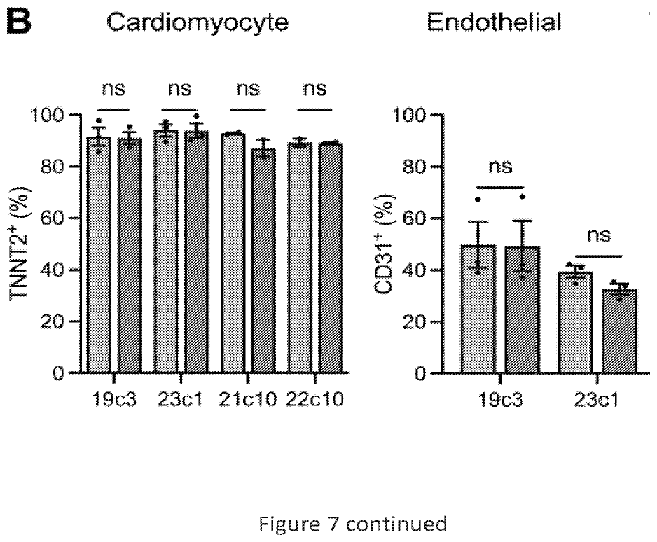

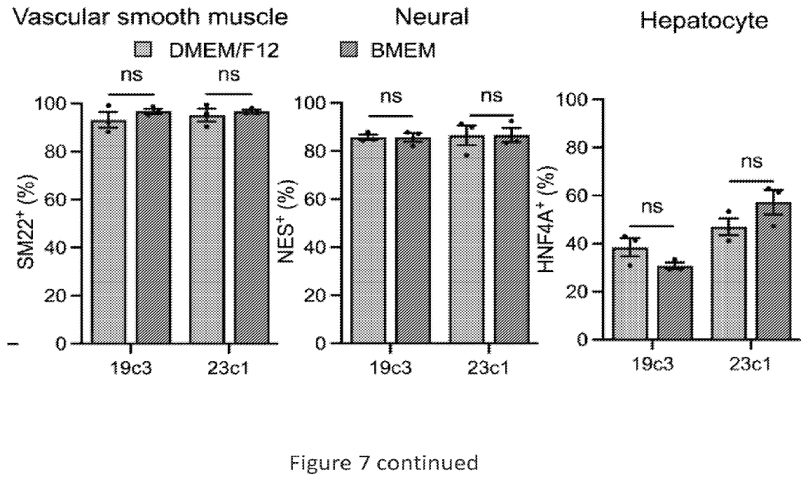

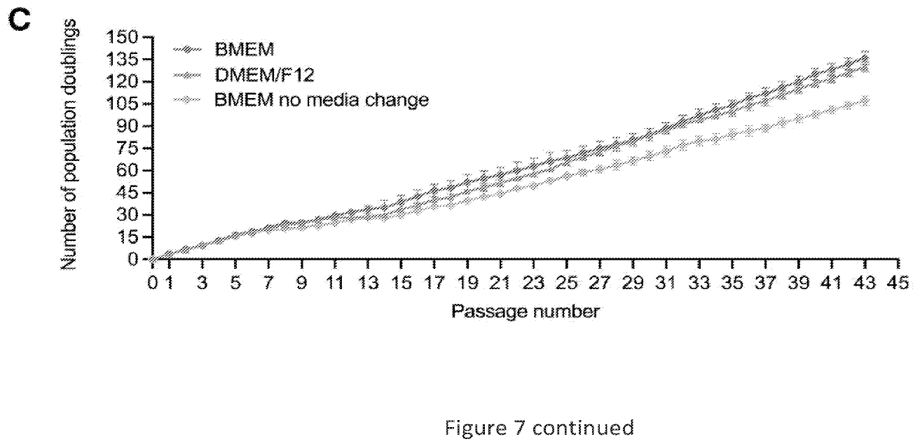

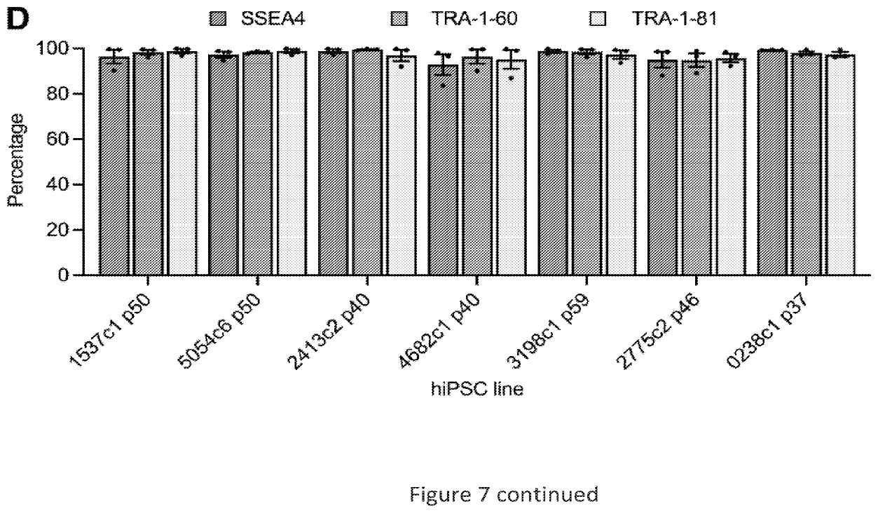

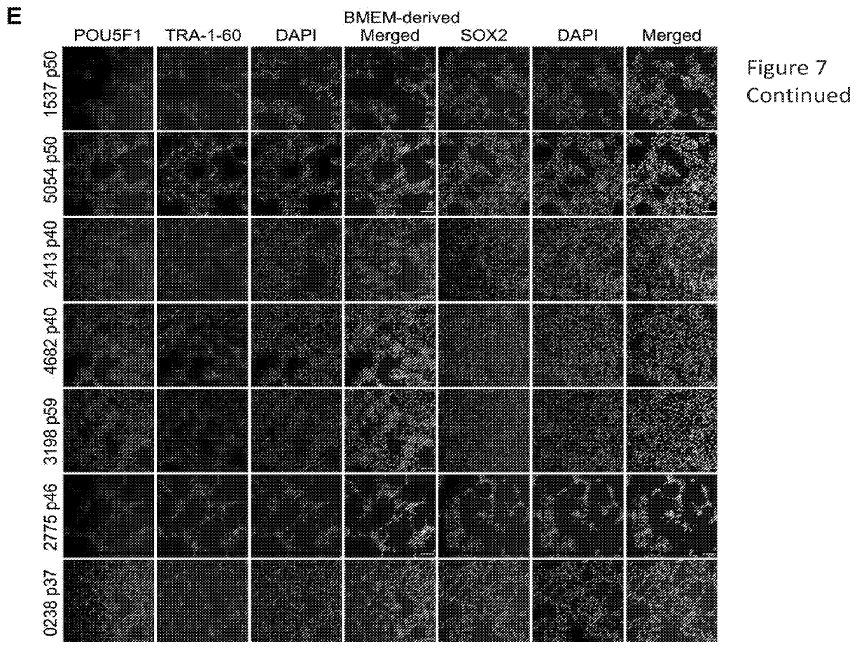

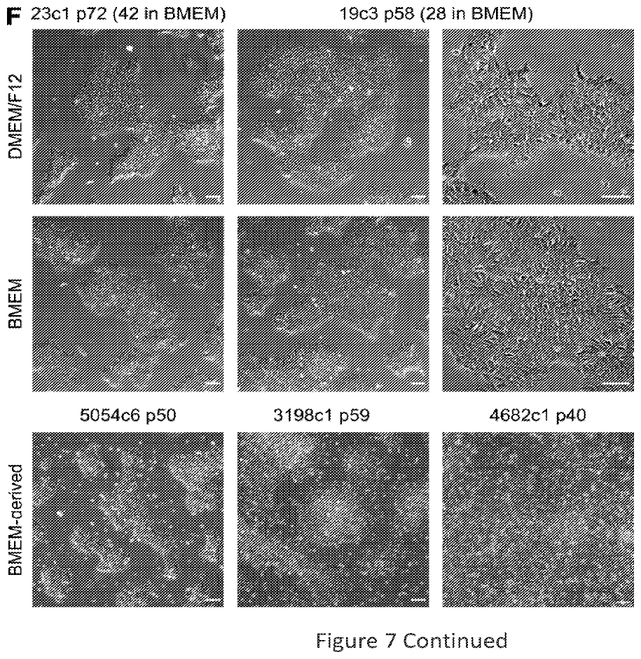

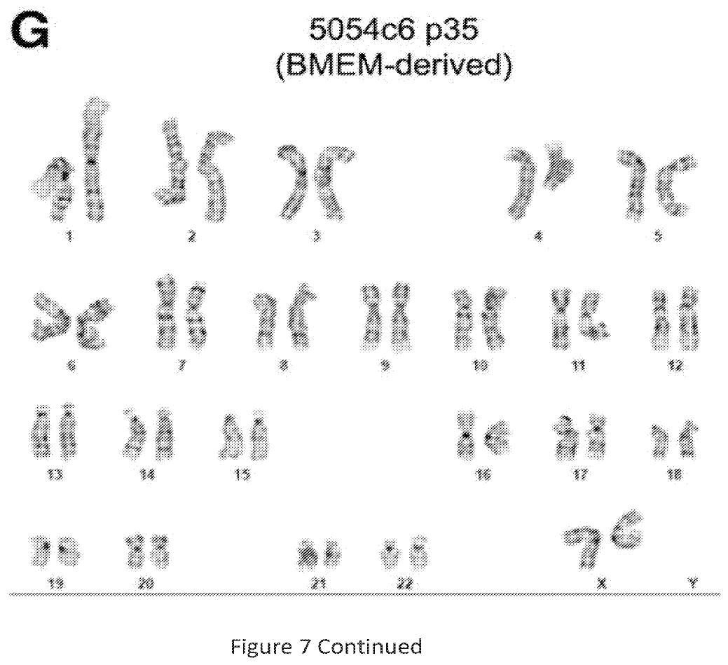

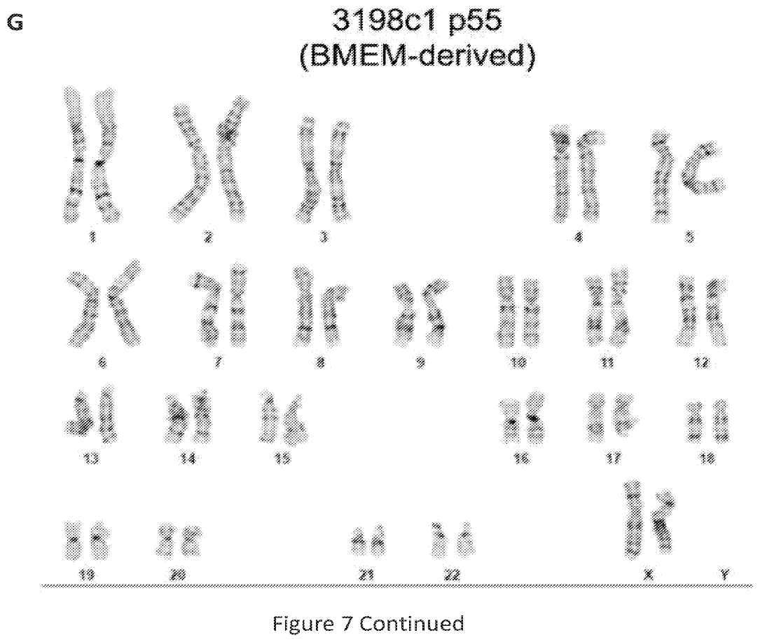

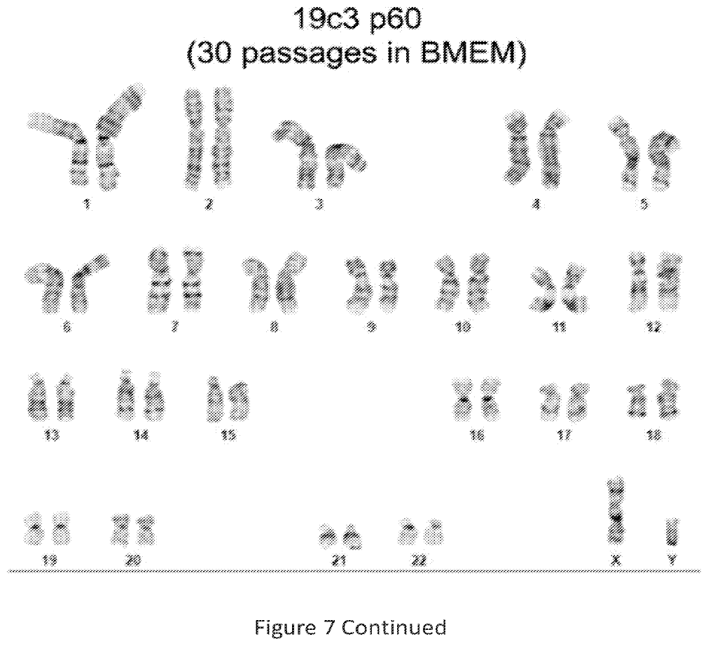

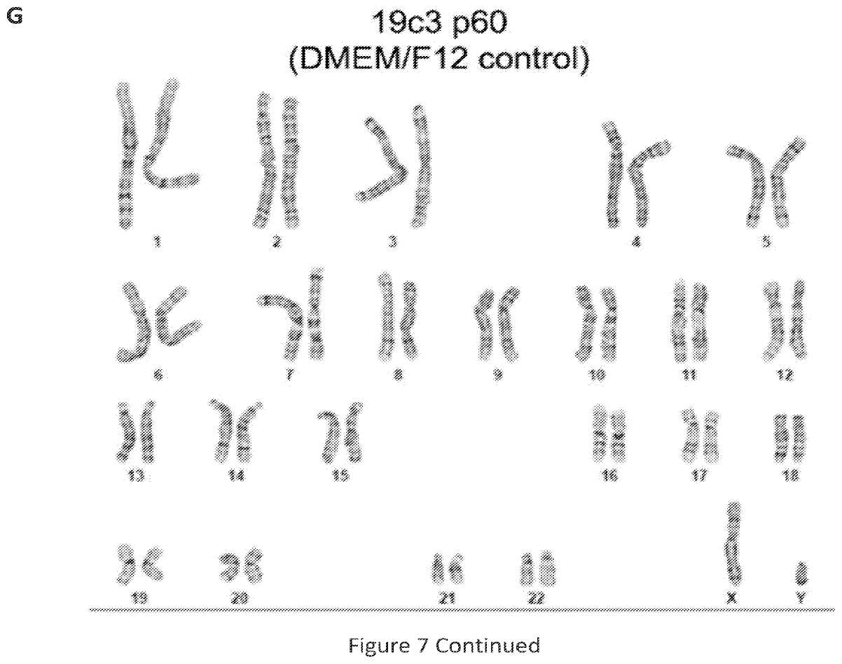

Next, hiPSC lines either cultured in BMEM for >30 passages or derived and cultured exclusively in BMEM were characterized, compared with those maintained in DMEM/F12-based medium. All lines were evaluated based on their undifferentiated cell marker expression and capacity to differentiate. Cells cultured long term in BMEM (minimum 25 passages) showed similar high-level expression of TRA-1-60 and SSEA4, markers of the undifferentiated state, assessed via flow cytometry (FIG. 7A). BMEM-cultured hiPSCs also showed similar capacity to differentiate into cardiomyocytes, endothelial, vascular smooth muscle, neural, and hepatocyte-like cells (FIG. 7B). BMEM-cultured cells showed higher, albeit non-statistically significant, growth rates compared with those cultured in DMEM/F12, when daily medium change was performed and only slightly slower growth when using a no medium change protocol (FIG. 7C).

Derivation of hiPSC Lines in BMEM

Whether BMEM was suitable for the derivation of hiPSC lines was then investigated, deriving seven lines exclusively in BMEM. BMEM-derived lines (>p37) presented typical high levels of expression of the undifferentiated cell markers SSEA4, TRA-1-60, and TRA-1-81 (FIG. 7D) and positive immunofluorescent staining for POU5F1, TRA-1-60, and SOX2 (FIG. 7E). Both BMEM-cultured and BMEM-derived hiPSC lines presented similar morphology, with BMEM cells slightly elongated and skinnier compared with those maintained in DMEM/F12 (FIG. 7F). This morphology remains after several passages, but cells still retain distinct hiPSC characteristics such as visible nucleoli, clearly identifiable nuclei, and white cell borders. As a final characterization step, both BMEM-cultured and BMEM-derived hiPSC lines, as those generated and maintained in DMEM/F12, were verified to present normal karyotypes even after >30 passages, suggesting that both media maintain similar levels of genomic stability (FIG. 7G).

Characterization of BMEM-Cultured hiPSC Lines on Commercially Available Extracellular Matrices

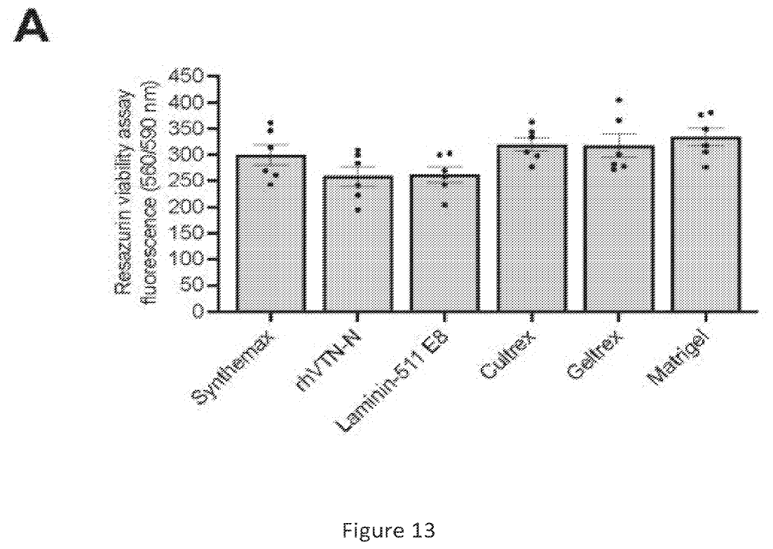

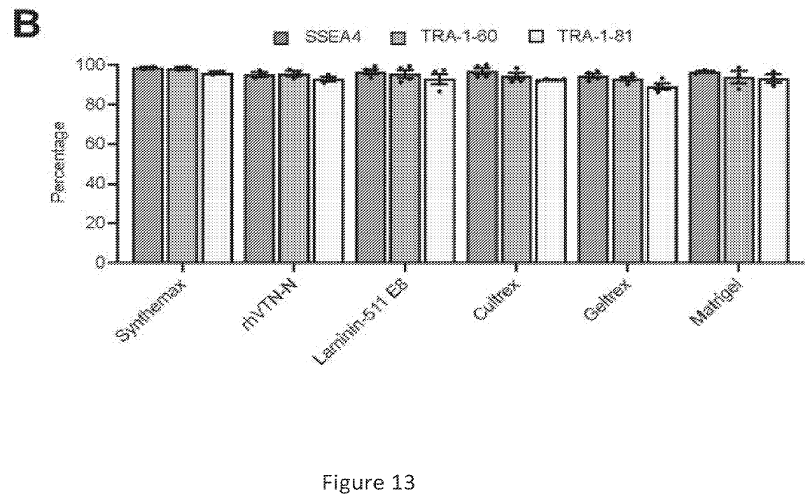

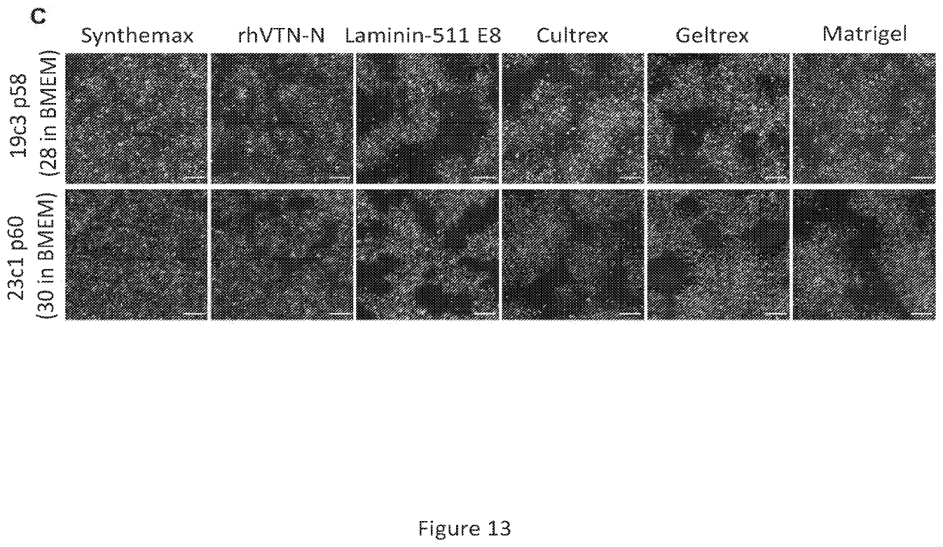

Finally, whether BMEM was suitable for hiPSC culture using different commercially available extracellular matrices (ECMs) in addition to standard Matrigel-based culture was investigated. BMEM-cultured lines were seeded on Synthemax II-SC (a vitronectin peptide), recombinant human vitronectin (rh-VTN-N), recombinant human laminin 511, Cultrex, Geltrex, and Matrigel, and cultured for five passages. Irrespective of the ECM, BMEM-cultured hiPSC lines presented similar levels of growth (FIG. 13A) and expression of undifferentiated markers (FIG. 13B). As expected, the different ECMs lead to a small change in morphology (FIG. 13C).

TABLE 1
Media formulation

| μM | | | | |
|---|---|---|---|---|
| BMEM | BME | MEM | DMEM | DMEM/F12 |

| | | | | |
|---|---|---|---|---|
| Gibco Cat. No. | 21010046 | 11095080 | 11965092 | 11320032 |

Amino Acids

| | | | | | |
|---|---|---|---|---|---|
| Glycine | 200 | | | 400 | 250 |
| L-Alanine | 50 | | | | 50 |
| L-Arginine HCl | 200 | 100 | 598 | 399 | 700 |
| L-Asparagine•H₂O | 50 | | | | 50 |
| L-Aspartic acid | 50 | | | | 50 |
| L-Cysteine | 100 | | | | 100 |
| HCl•H₂O | | | | | |
| L-Cystine 2HCl | | 51 | 99 | 201 | 100 |
| L-Glutamic acid | 50 | | | | 50 |
| L-Glutamine | 1000 | 1998 | 1998 | 3996 | 2498 |
| L-Histidine | | 52 | | | |
| L-Histidine | 100 | | 200 | 200 | 150 |
| HCl•H₂O | | | | | |
| L-Isoleucine | 200 | 198 | 396 | 800 | 415 |
| L-Leucine | 500 | 198 | 396 | 800 | 450 |
| L-Lysine HCl | 500 | 200 | 400 | 799 | 500 |
| L-Methionine | 100 | 50 | 101 | 201 | 116 |
| L-Phenylalanine | 100 | 100 | 194 | 400 | 215 |
| L-Proline | 150 | | | | 150 |
| L-Serine | 250 | | | 400 | 250 |
| L-Threonine | 500 | 201 | 403 | 798 | 449 |
| L-Tryptophan | 200 | 20 | 49 | 78 | 44 |
| L-Tyrosine | 200 | 115 | 231 | 462 | 248 |
| disodium salt | | | | | |
| hydrate | | | | | |
| L-Valine | 200 | 201 | 393 | 802 | 451 |

Vitamins

| | | | | | |
|---|---|---|---|---|---|
| Biotin (B₇) | 0.005 | 4.09 | | | 0.01 |
| Choline chloride | | 7.2 | 7.2 | 28.6 | 64.3 |
| Choline bitartrate | 200 | | | | |
| D-Pantothenic acid | 20 | 4.2 | 4.2 | 16.8 | 9.4 |
| hemicalcium salt | | | | | |
| (B₅) | | | | | |
| Folic acid (B9) | 1 | 2.3 | 2.3 | 9.1 | 6.0 |
| Nicotinamide (B₃) | 1 | 8.2 | 8.2 | 32.8 | 16.5 |
| Pyridoxal | | 4.9 | 4.9 | | |
| hydrochloride (B⁶) | | | | | |
| Pyridoxine | 5 | | | 19.5 | 9.7 |
| hydrochloride (B₆) | | | | | |
| Riboflavin (B₂) | 0.05 | 0.3 | 0.3 | 1.1 | 0.6 |
| Thiamine | | 3.0 | 3.0 | 11.9 | 6.4 |
| hydrochloride (B₁) | | | | | |
| Vitamin B₁₂ | | | | | 0.5 |
| myo-Inositol | 100 | 11.1 | 11.1 | 40.0 | 69.9 |

Inorganic Salts

| | | | | | |
|---|---|---|---|---|---|
| Calcium chloride | 500 | 1802 | 1802 | 1802 | 1051 |
| (CaCl₂) | | | | | |
| Copper(II) sulfate | | | | | 0.01 |
| pentahydrate | | | | | |
| (CuSO₄•5H₂O) | | | | | |
| Iron(III) nitrate | | | | 0.2 | 0.12 |
| nonahydrate | | | | | |
| (Fe(NO₃)₃•9H₂O) | | | | | |
| Iron(II) sulfate | 0.2 | | | | 1.50 |
| heptahydrate | | | | | |
| (FeSO₄•7H₂O) | | | | | |
| Magnesium | | | | | 301 |
| chloride (MgCl₂) | | | | | |
| Magnesium sulfate | 2000 | 811 | 811 | 811 | 406 |
| (MgSO₄) | | | | | |
| Potassium chloride | 2000 | 5366 | 5366 | 5366 | 4182 |
| (KCl) | | | | | |
| Sodium | 22500 | 26187 | 26187 | 44042 | 14284 |
| bicarbonate | | | | | |
| (NaHCO₃) | | | | | |
| Sodium chloride | 110000 | 116359 | 116359 | 109514 | 119704 |
| (NaCl) | | | | | |
| Sodium phosphate | | | | | 500 |
| dibasic | | | | | |
| (Na₂HPO₄) | | | | | |
| Sodium phosphate | 2000 | 1015 | 1015 | 906 | 453 |
| monobasic | | | | | |
| monohydrate | | | | | |
| (NaH₂PO4•H₂O) | | | | | |
| Zinc sulfate | 0.200 | | | | 2 |
| heptahydrate | | | | | |
| (ZnSO₄•7H₂O) | | | | | |

Other Components

| | | | | | |
|---|---|---|---|---|---|
| D-(+)-Glucose | 12500 | 5551 | 5551 | 24978 | 17490 |
| HEPES | | | | | 15000 |
| Hypoxanthine | | | | | 18 |
| Linoleic acid | | | | | 0.15 |
| Lipoic acid | | | | | 1 |
| Phenol red | | 28 | 28 | 42 | 23 |
| Phenol red sodium | 22.3 | | | | |
| salt | | | | | |
| Putrescine | | | | | 1 |
| dihydrochloride | | | | | |
| Sodium pyruvate | 750 | | | | 500 |
| Thymidine | | | | | 2 |
| Count | 39 | 30 | 29 | 32 | 52 |

TABLE 2
Media Components, related to FIGS. 1-5. Details of concentrations
and storage conditions of the stock solutions for the
individual DMEM/F12 and/or BMEM components.

| | | | Stock | | |
|---|---|---|---|---|---|
| MW | Component | CAS | (mg/mL) | Solubility | Storage |

Amino Acids

| | | | | | | |
|---|---|---|---|---|---|---|
| 75.07 | Glycine | 56-40-6 | 100.0 | Water | 4° | C. |
| 89.09 | L-Alanine | 56-41-7 | 100.0 | Water | 4° | C. |
| 210.66 | L-Arginine HCl | 1119-34-2 | 100.0 | Water | 4° | C. |
| 150.13 | L-Asparagine•H2O | 5794-13-8 | 100.0 | 0.5M HCl | 4° | C. |
| 133.1 | L-Aspartic acid | 56-84-8 | 50.0 | 1M HCl | 4° | C. |
| 175.64 | L-Cysteine HCl•H2O | 7048-04-6 | 50.0 | Water | 4° | C. |
| 313.22 | L-Cystine 2HCl | 30925-07-6 | 50.0 | 2M HCl | 4° | C. |
| 147.13 | L-Glutamic Acid | 56-86-0 | 100.0 | 1M HCl | 4° | C. |
| 146.14 | L-Glutamine | 56-85-9 | 29.2 | Water | 4° | C. |
| 209.63 | L-Histidine HCl•H2O | 5934-29-2 | 100.0 | Water | 4° | C. |
| 131.17 | L-Isoleucine | 73-32-5 | 100.0 | 1M HCl | 4° | C. |
| 131.17 | L-Leucine | 61-90-5 | 100.0 | 1M HCl | 4° | C. |
| 182.65 | L-Lysine HCI | 657-27-2 | 100.0 | Water | 4° | C. |
| 149.21 | L-Methionine | 63-68-3 | 100.0 | 0.5M HCl | 4° | C. |
| 165.19 | L-Phenylalanine | 63-91-2 | 100.0 | 1M HCl | 4° | C. |
| 115.13 | L-Proline | 147-85-3 | 100.0 | Water | 4° | C. |
| 105.09 | L-Serine | 56-45-1 | 100.0 | Water | 4° | C. |
| 119.12 | L-Threonine | 72-19-5 | 100.0 | 0.1M HCl | 4° | C. |
| 204.23 | L-Tryptophan | 73-22-3 | 100.0 | 1M HCl | 4° | C. |
| 225.15 | L-Tyrosine disodium | 69847-45-6 | 100.0 | Water | 4° | C. |
| | salt hydrate | | | | | |
| 117.15 | L-Valine | 72-18-4 | 100.0 | 1M HCl | 4° | C. |

Vitamins

| | | | | | |
|---|---|---|---|---|---|
| 244.31 | Biotin | 58-85-5 | 0.01 | Water | RT, |
| | | | | | protect |
| | | | | | from light |
| 253.25 | Choline bitartrate | 87-67-2 | 50.0 | Water | RT, |
| | | | | | protect |
| | | | | | from light |
| 238.27 | D-Pantothenic acid | 137-08-6 | 1.0 | Water | 4° C., |
| | hemicalcium salt | | | | protect |
| | | | | | from light |
| 441.4 | Folic acid | 59-30-3 | 5.0 | 2:1 | RT, |
| | | | | water:sodium | protect |
| | | | | bicarbonate | from light |
| 122.12 | Nicotinamide | 98-92-0 | 1.0 | Water | RT |
| | | | | | protect |
| | | | | | from light |
| 205.64 | Pyridoxine | 58-56-0 | 10.0 | Water | 4° C., |
| | hydrochloride | | | | protect |
| | | | | | from light |
| 376.36 | Riboflavin | 83-88-5 | 0.2 | 49 mL water & | RT |
| | | | | 1 mL 1N NaOH | protect |
| | | | | | from light |
| 337.27 | Thiamine | 67-03-8 | 1.0 | Water | 4° C., |
| | hydrochloride | | | | protect |
| | | | | | from light |
| 1355.37 | Vitamin B12 | 68-19-9 | 5.0 | Water | 4° C., |
| | | | | | protect |
| | | | | | from light |
| 180.16 | myo-Inositol | 87-89-8 | 100.0 | Water | 4° C., |
| | | | | | protect |
| | | | | | from light |

Inorganic Salts

| 110.98 | Calcium chloride | 10043-52-4 | 700.0 | Water | RT | |
|---|---|---|---|---|---|---|
| 249.69 | Copper(II) sulfate | 7758-99-8 | 0.01 | Water | RT | |
| | pentahydrate | | | | | |
| 404 | Iron(III) nitrate | 7782-61-8 | 0.1 | Water | RT | |
| | nonahydrate | | | | | |
| 278.01 | Iron(II) sulfate | 7782-63-0 | 1.0 | Water | RT | |
| | heptahydrate | | | | | |
| 95.21 | Magnesium chloride | 7786-30-3 | 250.0 | Water | RT | |
| 120.37 | Magnesium sulfate | 7487-88-9 | 250.0 | Water | RT | |
| 74.55 | Potassium chloride | 7447-40-7 | 250.0 | Water | RT | |
| 58.44 | Sodium chloride | 7647-14-5 | — | — | Add as | |
| | | | | | powder | |
| 141.96 | Sodium phosphate | 7558-79-4 | 71.0 | Water | RT | |
| | dibasic | | | | | |
| 137.99 | Sodium phosphate | 10049-21-5 | 500.0 | Water | RT | |
| | monobasic | | | | | |
| | monohydrate | | | | | |
| 287.56 | Zinc sulfate | 7446-20-0 | 10.0 | Water | RT | |
| | heptahydrate | | | | | |
| 180.16 | D-(+)-Glucose | 50-99-7 | — | — | Add as | |
| | | | | | powder | |
| 238.3 | HEPES | 7365-45-9 | 500.0 | Water | RT | |
| 110.04 | Sodium Pyruvate | 113-24-6 | 250.0 | Water | 4° | C. |

Other Components

| 136.11 | Hypoxanthine | 68-94-0 | 20.0 | 2:1 formic | RT | |
|---|---|---|---|---|---|---|
| | | | | acid:water | | |
| 280.45 | Linoleic acid | 60-33-3 | 0.5 | 1:1 | −20° | C. |
| | | | | ethanol:DPBS | | |
| 206.33 | Lipoic acid | 1077-28-7 | 50.0 | Ethanol | RT | |
| 376.36 | Phenol red sodium | 34487-61-1 | 100.0 | Water | 4° | C. |
| | salt | | | | | |
| 161.07 | Putrescine | 333-93-7 | 100.0 | Water | 4° | C. |
| | dihydrochloride | | | | | |

| | | | | | |
|---|---|---|---|---|---|
| 84.01 | Sodium bicarbonate | 144-55-8 | 90.0 | Water | RT |
| 242.23 | Thymidine | 50-89-5 | 1.0 | Water | RT |

### Discussion

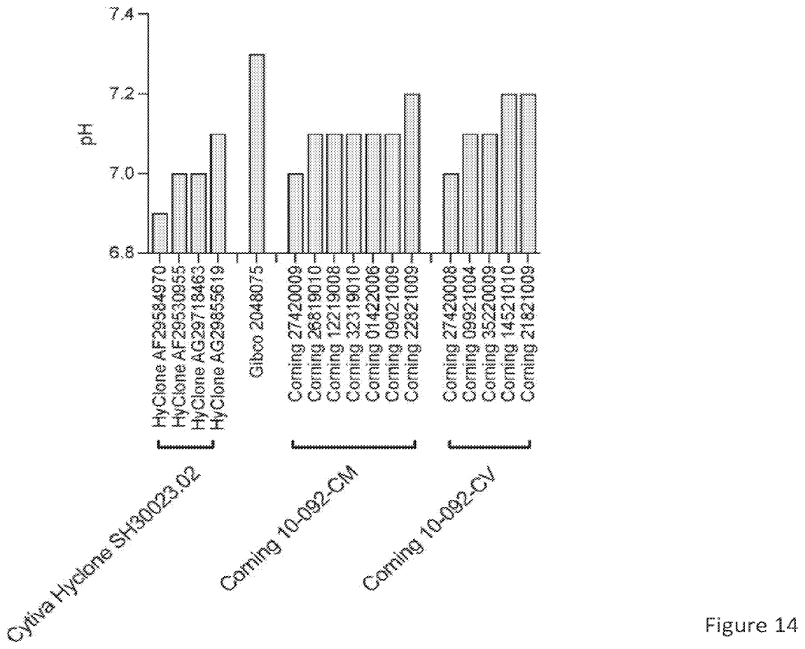

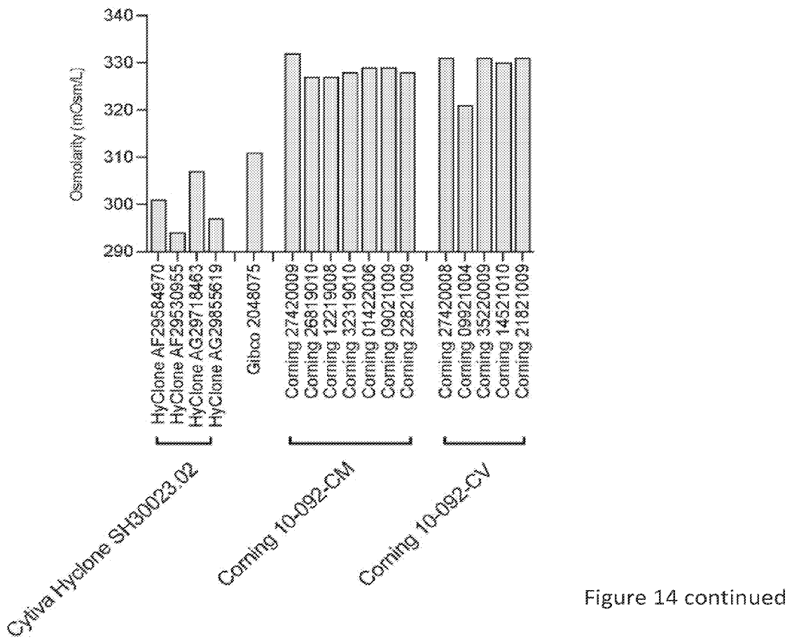

Stable and fast pluripotent cell growth is essential for all aspects of hiPSC use and especially integral to efficient differentiation (Burridge et al., 2014). Basal medium selection has an integral role in hiPSC growth in this process and it is therefore somewhat cryptic that the suitability of DMEM/F12 has not been questioned previously. In our prior B8 publication (Kuo et al., 2020), we developed simple methods for comparative analysis of manipulations to hiPSC growth conditions, allowing us here to study multiple components that influence growth that has not been feasible previously. Importantly, although there are many companies that supply DMEM/F12, the exact formula used varies slightly and the specifications for pH and osmolarity are typically wide (FIG. 14). Because of this variability, some suppliers and even individual batches from suitable suppliers are suboptimal for hiPSC culture. Indeed, in our own lab we have traced prior issues with slow growth to specific batches of commercial basal media that are within specifications, but out of our defined appropriate range for hiPSC culture. When using BMEM, we specifically define the pH and osmolarity eliminating this source of variability.

From the outset, the development of BMEM was intended to mimic the development of Eagle's minimal essential medium, with the elimination of as many components as possible both to provide insight into the nutritional requirements for hiPSCs and to simplify the generation of this medium in-house. This contrasts with the trend toward creating vastly more complex media for the culture of cancer cell lines including the formulations of SMEM (Tardito et al., 2015), HPLM (Cantor et al., 2017), and Plasmax (Vande Voorde et al., 2019), with the rationale that historic media do not recapitulate the complex nutritional environment of the tumor (Ackermann and Tardito, 2019). A similar rationale has been applied to the culture of neurons with the “BrianPhys” formulation (Bardy et al., 2015). The formulations are theoretically based on the concentrations of metabolites in serum or tumor interstitial fluid, yet each still requires numerous additional components derived from other basal media formulations as well as supplementation with FBS (SMEM and HPLM at 10%, Plasmax at 2.5%), suggesting that merely recapitulating the in vivo nutritional environment is not appropriate for in vitro culture.

BMEM lacks several components that would have been originally suspected to be essential in a chemically defined medium for hiPSC culture. Out of those, linoleic acid would seem to be the most irreplicable as it, and linolenic acid, are considered essential fatty acids and a precursor to many other fatty acids. This raises the question of how BMEM-cultured cells are generating their cell membranes without the lipid contribution to their phospholipid bilayers. Fatty acid synthase (FASN) was found to be one of the most highly expressed genes in both BMEM- and DMEM/F12-cultured cells, expressed at similar levels to POU5F1 and, along with the presence of abundant acetyl-CoA and acetyl-CoA carboxylase (ACACA), likely allows hiPSCs to synthesize fatty acids via de novo lipogenesis.

In typical culture, three methodologies were used to reduce the risk of karyotype instability: clump rather than single-cell passaging, inclusion of a ROCK inhibitor (thiazovivin), and use of hypoxic (5% (2) incubators (Thompson et al., 2020). Three hiPSC lines derived or cultured in BMEM are demonstrated to have no detectable karyotypic abnormalities by G-banded karyotype. These data are pre-liminary and further in-depth analysis will be required to compare genomic stability of hiPSCs in various media.

Finally, BMEM was developed with both daily medium change and an extreme no medium change feeding schedule. This was done to identify components that are limiting when skipping medium change days or weekends. Overall, the no medium change data did not identify any additional requirements except for the need for sodium phosphate. Thus, a no change strategy, along a monolayer growth method, is compatible with hiPSC culture long term (44 passages), although it likely has negative effects on differentiation that would be expected are reversible upon resuming more frequent medium changes.

In summary, the nutrient requirements for hiPSCs have been assessed, providing a medium formulation that is simple and cost-effective to make, and that provides valuable insight into the nutritional requirements of human pluripotent cells that can be applied to the development of human pluripotent media. Importantly, BMEM pushes hiPSCs into a higher pluripotent state, potentially providing enhanced stability and differentiation reproducibility as well as providing a new nutrition-based avenue to study the human pluripotency.

### Experimental Procedure

Human induced pluripotent cell culture: BMEM consists of BMEM basal medium and BMEM supplement. BMEM basal medium consists of a 253 salt solution, 2003 vitamin solution (with 30 mL/L 1 N NaOH), and a 2003 amino acid solution (with 80 mL/L 12 N HCl) (pH 7.1). These are added to ultrapure water and filter sterilized. BMEM supplement consists of 5 mg/mL E. coli-derived recombinant human insulin (Gibco), 200 mg/mL L-ascorbic acid 2-phosphate trisodium salt (AA2P, Wako), 5 mg/mL Oryza sat-iva-derived recombinant human transferrin (Optiferrin, InVitria), 20 ng/mL sodium selenite (Sigma), 40 ng/mL recombinant human FGF2-G3 (made in-house), 0.1 ng/mL recombinant human TGFb3 (E. coli-derived, Qkine), 0.1 ng/mL recombinant human NRG1 (E. coli-derived, Shenodoah Biotechnology). Cells were routinely maintained in this medium on 1:800 diluted growth factor reduced Matrigel (Corning) diluted in a BMEM basal at 2 mL per well of a 6-well plate or equivalent. Matrigel-coated plates were kept in CO₂ incubators at 37° C. for up to 1 month. BMEM was supplemented with 2 mM thiazovivin (LC Labs), hereafter referred to as BMEM-T, for the first 24 h after passage. For standard culture, cells were passaged at a ratio of 1:20 every 4 days using 0.5 mM EDTA (Invitrogen) diluted in DPBS^{-/-} (without Ca²⁺ and Mg²⁺, Corning), after achieving ˜70%-80% confluence. Cell lines were used between passages 20 and 100. Throughout, pH was adjusted immediately after making the medium at room temperature and atmospheric oxygen and carbon dioxide levels but before filter sterilization. pH was adjusted with 12 N HCl or 1 N NaOH (both from Fisher) and measured using a SevenCompact pH meter (MettlerToledo). Osmolarity was measured using an osmometer (Advanced Instruments).

All pluripotent and reprogramming cell cultures were maintained at 37° C. in Heracell VIOS 160i direct heat humidified incubators (Thermo Scientific) with 5% CO₂ and 5% O₂. Differentiation cultures were maintained at 5% CO₂ and atmospheric (˜21%) 02. All cultures (pluripotent and differentiation) were maintained with 2 mL medium per 9.6 cm² of surface area or equivalent. All media was used at 4° C. and was not warmed to 37° C. before adding to cells for simplicity and due to concerns of the temperature stability of the FGF2 (Chen et al., 2012). We have found no detectable effects on cell growth from using cold media. All cultures were routinely tested for mycoplasma using a MycoAlert PLUS Kit (Lonza) and a 384-well Varioskan LUX (Thermo Scientific) plate reader. hiPSC lines 19c3 and 23c1 were used throughout (Kuo et al., 2020).

Resazurin assay: Resazurin sodium salt (B21187.06, Thermo) was diluted to 5 mg/mL in sterile Milli-Q ultrapure water and 600 μL aliquots were made and stored at −20° C. One aliquot was added to 14.4 mL of water to generate a 10× stock. Stock was diluted in BMEM to 1×, and 1 mL was added to each well of a 12-well plate and incubated at 37° C. for 2.5 h. Plates were read on a Varioskan Lux (Thermo) in fluorescent mode using top read and an excitation wavelength of 560 nm and an emission wavelength of 590 nm.

Basal media comparisons: Twelve basal media were purchased from Gibco: BME (21010046), MEM (11095080), DMEM (11965092), IMDM (12440053), MEMa (12560156), F12 (11765054), DMEM/F12 (11320032), RPMI 1640 (11875093), M199 (11150059), L15 (11415064), and F10 (11550043). Each was normalized to the DMEM/F12 levels of L-glutamine, D-glucose, sodium pyruvate, sodium bicarbonate, pH, and osmolarity.

hiPSC differentiation: hiPSCs were differentiated to cardiomyocytes, endothelial cells, neural progenitors, and hepatocytes using established protocols. These lines were used in this publication: 19c3, 21c10, 22c10, 23c1, 1537c1, 5054c6, 6955c3, 4682c1, 4138c2, 3198c1, 2775c2, and 0238c1. 19c3 and 23c1 were previously reported (Kuo et al., 2020). Protocols were approved by the Northwestern University Institutional Review Boards. With informed written consent, ˜9 mL of peripheral blood was taken from each volunteer and stored at 4° C., samples were transferred to LeucSep tubes (Greiner) filled with Histopaque-1077 (Sigma). 1×106 isolated peripheral blood mononuclear cells (PMBC) were grown in 24-well tissue culture-treated plates (Greiner) in 2 mL of SFEM II (Stem Cell Technologies) supplemented with 10 ng/ml IL3, 50 ng/mL SCF (KITLG) (all Peprotech), 2 U/mL EPO (Calbiochem), 1 μM dexamethasone (Sigma) (Chou et al, 2015). 50% medium was changed every other day. After 12-14 days of growth (once a lawn of cells was achieved). 6×104 cells were transferred to a well of a 24-well plate in 500 μL of SFEM II with growth factors supplemented with CytoTune-iPS 2.0 Sendai Reprogramming Kit viral particle factors (Gibco) (Fujie et al., 2014; Fusaki et al., 2009) diluted to 5% ( 1/20th) of the manufacturer's recommendation. 100% media was changed after 24 h by centrifugation (300×g, 4 min) to 2 mL fresh SFEM II with growth factors, and cells were transferred to one well of a 6-well plate coated with 1:800 Matrigel. 50% medium (1 mL) was changed gently every other day. On day 12 after transduction (once colonies were visible), 100% of medium was changed to BMEM medium (1537c1, 5054c6, 6955c3, 4682c1, 4138c2, 3198c1, 2775c2, and 0238c1) or DMEM/F12 supplemented with BMEM supplement (21c10, 22c10, 23c1, and 26c3). Medium was then changed every day. At day 17-20 individual colonies were picked in to a Matrigel-treated 12-well plate (one colony per well).

hiPSC derivation: These lines were used in this publication: 19c3, 21c10, 22c10, 23c1, 1537c1, 5054c6, 6955c3, 4682c1, 4138c2, 3198c1, 2775c2, and 0238c1 19c3 and 23c1 were reported previously (Kuo et al., 2020). hiPSCs were derived as described previously (Kuo et al., 2020).

Cardiac differentiation: Differentiation into cardiomyocytes was performed according to our RBAI protocol (Magdy et al., 2022). Briefly, hiPSCs were split at a 1:25 ratio using 0.5 mM EDTA as above and grown in BMEM for 4 days reaching ˜70-80% confluence. At the start of differentiation (day 0), BMEM medium was changed to R6C consisting of RPMI 1640 (10-040-CM, Corning) supplemented with 6 μM of the glycogen synthase kinase 3-β inhibitor CHIR99021 (C-6556, LC Labs). On day 1, medium was changed to RPMI alone. On day 2, medium was changed to RBAI consisting of RPMI supplemented with 2 mg/ml of bovine serum albumin (BSA, A0100, GenDEPOT), 200 μg/mL of L-ascorbic acid-2-phosphate (AA2P; 321-44823, Wako) and 2 μM of the Wnt inhibitor Wnt-C59 (orb 181132, Biorbyt). Medium was then changed on day 4 and every other day for RBAI, consisting of RPMI supplemented with 0.5 mg/mL BSA, 200 μg/mL AA2P, and 5 g/mL of human recombinant insulin (A11382ij, Gibco). Contracting cells were noted from day 7. On day 15, cardiomyocytes were dissociated using DPBS for 20 min at 37° C. followed by 1:200 Liberase TH (5401151001, Roche) diluted in DPBS for 20 min at 37° C., centrifuged at 300×g for 5 min, and filtered through a 100 μm cell strainer (Fisherbrand) and analyzed.

Endothelial differentiation: Differentiation into endothelial cells was performed according to our modified version of protocols previously described (Patsch et al., 2015; Toyohara et al., 2020). Briefly, hiPSCs were split at a 1:25 ratio using 0.5 mM EDTA as above and grown in BMEM or DMEM/F12 for 4 days reaching ˜70-80% confluence. At the start of differentiation (day 0), BMEM medium was changed to R6C, consisting of RPMI 1640 (10-040-CM, Corning) supplemented with 6 μM of the glycogen synthase kinase 3-β inhibitor CHIR99021 (C-6556, LC Labs). On day 2, medium was changed to RBAI-VFF consisting of RPMI supplemented with 2 mg/mL of bovine serum albumin (BSA, A0100, GenDEPOT), 200 μg/mL of L-ascorbic acid-2-phosphate (AA2P; 321-44823, Wako), 5 μg/ml of human recombinant insulin (A11382ij, Gibco), 100 ng/ml of VEGFA165 (Peprotech, 100-20), 10 μM forskolin (LC Labs, F-9929), and 100 ng/mL of FGF2-G3 (made in-house). On day 5, endothelial cells were dissociated using Accutase (25058C1, Corning) for 6 min at 37° C., centrifuged at 300×g for 5 min and analyzed by flow cytometry.

Smooth muscle differentiation: Differentiated to vascular smooth muscle cells was performed according to modified version of protocols previously described (Toyohara et al., 2020). Briefly, hiPSCs were split at a 1:25 ratio using 0.5 mM EDTA as above and grown in BMEM or DMEM/F12 for 4 days reaching ˜70-80% confluence. At the start of differentiation (day 0), BMEM medium was changed to R6C, consisting of RPMI 1640 (10-040-CM, Corning) supplemented with 6 UM of the glycogen synthase kinase 3-β inhibitor CHIR99021 (C-6556, LC Labs). On day 2, medium was changed to RBAI-PA consisting of RPMI supplemented with 2 mg/mL of bovine serum albumin (BSA, A0100, GenDEPOT), 200 μg/mL of L-ascorbic acid-2-phosphate (321-44823, Wako), 5 g/mL of human recombinant insulin (A11382ij, Gibco), 12.5 ng/ml of PDGF-BB (100-14B, Peprotech), and 12.5 ng/ml of Activin A (120-14E, Peprotech). On day 4, vascular smooth muscle cells were dissociated using Accutase (25058C1, Corning) for 6 min at 37° C., centrifuged at 300×g for 5 min, and analyzed by flow cytometry.

Neuronal differentiation: Differentiation into neural progenitor cells was performed according to modified version of previously published protocols based on dual SMAD inhibition (Zhong et al., 2018). Briefly, hiPSCs were split at a 1:2.5, 1:5, and 1:10 ratio using 0.5 mM EDTA as above and grown in BMEM or DMEM/F12 overnight. On the first day of differentiation (day 0), medium was changed to neural induction medium: 50% DMEM/F12 (10-090-CV, Corning), 50% Neurobasal Medium (21103-049, Gibco), supplemented with 1:100 nonessential amino acids (made in-house), 2 mM L-glutamine (G3126, Sigma), 1:100 N2 supplement (17502048, Gibco), 1% B27 supplement minus vitamin A (v/v, 12587010, Gibco), and 10 μM SB431542 (S-7800, LCLabs), 0.2 μM LDN-193189 (HY-12071, MedChem Express). Media was changed every day. On day 8, cells were dissociated with TrypLE for 5 min at 37° C., centrifuged at 300×g for 5 min, and analyzed by flow cytometry.

Hepatocyte differentiation: Differentiation into hepatocytes was performed according to our modified version of protocols previously described (Peters et al., 2016). hiPSCs were split with 0.5 mM EDTA as above and grown in BMEM or DMEM/F12 so that they are approximately 70% confluent 2 days later. On the first day of differentiation (day 0), medium was changed to DE medium: RPMI 1640 (10-040-CM, Corning) supplemented with 1% B27 minus insulin (v/v, A1895601, Gibco), 100 ng/mL Activin A (120-14E, Peprotech), and 3 μM CHIR99021 (C-6556, LC Laboratories). On day 4, medium was changed to HE medium: RPMI 1640 supplemented with 1% B27 minus insulin, 5 ng/mL FGF2-G3 (made in-house), 20 ng/mL BMP4 (120-05ET, Peprotech), and 0.5% DMSO (v/v, BP231-100, Fisher). On day 9, medium was changed to IMH medium: RPMI 1640 supplemented with 1% B27 minus insulin, 20 ng/ml HGF (100-39H, Peprotech), and 0.5% DMSO. Media was changed daily during differentiation at 24 h intervals. On day 10, cells were dissociated with TrypLE for 5 min at 37° C., centrifuged at 300×g for 5 min, and analyzed by flow cytometry.

Immunofluorescent staining: hiPSCs were dissociated with 0.5 mM EDTA and passaged onto Matrigel-coated 96-well Clear Flat Bottom TC-treated Culture Micro-plates (Falcon) into BMEM supplemented with thiazovivin for 24 h. After 24 h, medium was changed to BMEM and changed every 24 h until day 4 and stained using typical protocols.

Flow cytometry: For live cell staining, hiPSCs were dissociated using TrypLE Express (Gibco) for 3 min at 37° C. and endothelial cells were dissociated using Accutase (Corning) for 5 min at 37° C. For fixed cell staining, cardiomyocytes were dissociated using Liberase TH and DPBS as described previously, smooth muscle cells were dissociated with Accutase, and neural and hepatocyte-like cells were dissociated with TrypLE. Cells were stained using typical protocols. All cells were analyzed using a CytoFLEX (Beckman Coulter) with CytExpert 2.2 software and the acquired measurements were analyzed using the FlowJo v.10.8.2 software.

TABLE 3
List of antibodies and isotypes

| | Catalog | Lot | |
|---|---|---|---|
| Antibody | Number | Number | Supplier |
| SSEA-4-488 | 560308 | 1011169 | BD Biosciences |
| TRA-1-60-488 | 560173 | 5261629 | BD Biosciences |
| TRA-1-81-488 | 560174 | 1215142 | BD Biosciences |
| CD31-488 | 558068 | 6064654 | BD Biosciences |
| Isotype mouse IgG₃- | 563636 | 7128849 | BD Biosciences |
| 488 | | | |
| Isotype mouse IgM- | 562409 | 4128849 | BD Biosciences |
| 488 | | | |
| Isotype mouse IgG₂ₐ- | 557703 | 7089951 | BD Biosciences |
| 488 | | | |
| Mouse monoclonal | 565744 | 1117767 | BD Biosciences |
| IgG₁ TNNT2-647 | | | |
| Rabbit polyclonal | ab14106 | lotGR3247382-1 | Abcam |
| SM22/TAGLN | | | |
| Mouse monoclonal | 560393 | 1263493 | BD Biosciences |
| IgG₁ NESTIN-647 | | | |
| Mouse monoclonal | IC4605R | 1696772 | R&D Systems |
| IgG_{2B} HNF4A-647 | | | |
| AlexaFluor goat | A21245 | 1837984 | Invitrogen |
| anti-rabbit IgG-647 | | | |

Extracellular matrix assessment: Plates were coated 2 mL per well of a 6-well plate or equivalent. Coated plates were kept in CO2 incubators at 37° C. unless otherwise stated. hiPSC lines were cultured BMEM or DMEM/F12 on 6 different commercially available extracellular matrices: Synthemax II-SC (3535, Corning, 1:100 in water, 2 h at RT, aspirate and keep dry plates at RT), Vitronectin (VTN-N) Recombinant Human Protein, Truncated (A14700, Gibco, 1:50 in DPBS+/+), ECMatrix-511 ES Laminin Substrate ready-to-use (CC160, MilliporeSigma, 1:1), Cultrex UltiMatrix Reduced Growth Factor BME (BME001-05, Bio-Techne, 1:800 in Matrigel diluent), Geltrex (A14132-02, Gibco, 1:800 in Matrigel diluent), and Matrigel Growth Factor-Reduced (354230, Corning). hiPSCs were cultured for 5 passages in either DMEM/F12 or BMEM. Cells were then analyzed for morphology via phase contrast imaging, flow cytometry, or growth.

Metabolite consumption: hiPSCs were cultured in either BMEM, BMEM without non-essential amino acids, or DMEM/F12 with and without daily medium change. On the second day post seeding, cells incubated in TrypLE for 2 min in 37° C., and counted. Following cell count, cells were centrifuged at 300×g for 5 min, and pellets were washed twice with 5 mL of ice-cold DPBS^{-/-}, with another centrifugation round in between washes. Pellets were then resuspended for lysing in 1 mL of 80% (vol/vol) methanol cooled to −80° C., and incubated in −80° C. freezer for 5 min. Following the incubation, lysates were vortexed thrice for 1 minute, while being kept in −80° C. freezer between vortex rounds. Tubes were then moved to −20° C. overnight to precipitate proteins. On the following day, samples were vortexed for 30 sec, then centrifuged at 20,000×g for 15 min at 4° C. Metabolite containing supernatant was transferred to a 1.5 ml conical tube and transferred to the Northwestern Metabolomics Core in dry ice, where they were dried before further processing. Samples were resuspended in 10 μL per 100,000 cells and analyzed by high-performance liquid chromatography and high-resolution mass spectrometry, and tandem mass spectrometry (HPLC-MS/MS) as previously reported (Weinberg et al., 2019). Specifically, the system consisted of a Thermo Q-Exactive in line with an electrospray source and an Ultimate3000 (Thermo) series HPLC consisting of a binary pump, degasser and auto-sampler outfitted with an Xbridge amide column (Waters; dimensions 4.6 mm×100 mm and a 3.5-μm particle size). The mobile phase A contained 95% (v/v) water, 5% (v/v) acetonitrile, 20 mM ammonium hydroxide, 20 mM ammonium acetate, pH 9.0; B was 100% acetonitrile. The gradient was as follows: 0-1 min, 15% A; 18.5 min, 76% A, 18.5-20.4 min, 24% A; 20.4-20.5 min, 15% A; 20.5-28 min, 15% A with a flow rate of 400 μL/min. The capillary of the electron spray ionization (ESI) source was set to 275° C., with sheath gas at 45 arbitrary units, auxiliary gas at 5 arbitrary units and the spray voltage at 4.0 kV. In positive/negative polarity switching mode, an m/z scan range from 70 to 850 was chosen and MS1 data was collected at a resolution of 70,000. The automatic gain control (AGC) target was set at 1×10⁶ and the maximum injection time was 200 msec. The top 5 precursor ions were subsequently fragmented, in a data-dependent manner, using the higher energy collisional dissociation cell set to 30% normalized collision energy in MS2 at a resolution power of 17,500. Sample volumes of 10 μL containing 100,000 cells were injected. Data acquisition and analysis were carried out by Xcalibur 4.0 software and Tracefinder 2.1 software, respectively (both from Thermo). Peak area results were input to https://www.metaboanalyst.ca for further analysis (Pang et al., 2022). Data were normalized using the ratio between total ion count (TIC) and the lowest TIC in the sample batch and scaled using auto scaling.

RNA-seq: Five hiPSC lines were cultured in BMEM or DMEM/F12 for a minimum of five passages before first RNA extraction. On the day of passage, a minimum of three wells for each condition were selected. 5 hiPSC lines were cultured in BMEM or DMEM/F12 for a minimum of 5 passages before first RNA extraction. On the day of passage, a minimum of 3 wells for each condition were selected, and, from them, RNA was extracted using 150 μL/well of TRIzol Reagent (Thermo, 15596026). RNA was then purified using the Direct-zol RNA MicroPrep kit (Zymo, R2062) including on-column DNase digestion to remove genomic DNA. Samples were quantified using a Thermo Scientific NanoDrop 8000 and passed QC and were then shipped in dry ice for library preparation and sequencing.

Paired-end (100 bp) RNA-sequencing on hiPSCs was performed using the DNBSEQ sequencing platform at Beijing Genomics Institute. Total RNA was extracted and oligo dT beads were used to enrich mRNA with poly A tail using DNBSEQ Eukaryotic strand-specific mRNA library protocol. mRNA molecules were fragmented into small pieces and the fragmented mRNA was synthesized into first strand cDNA using random primers. The second strand cDNA was synthesized using dUTP instead of dTTP. The synthesized cDNA was subjected to end-repair and 3′ adenylated and adaptors were ligated to the ends of these 3′ adenylated cDNA fragments. The U-labelled second-strand template was digested with Uracil-DNA-Glycosylase (UDG) and PCR amplification was performed. Following library quality control and circularization, the library was amplified to make DNA nanoball (DNB) and sequenced on DNBSEQ platform.

Using SOAPnuke (Chen et al., 2018), raw fastq files were processed to trim/remove adaptors, low-quality reads, and N reads. Clean sequencing reads were mapped to the GRCh38 reference genome using STAR (Dobin et al., 2013) and counted using RSEM (Li and Dewey, 2011). Genes with less than 10 counts across all 30 samples were excluded from subsequent analysis. Following variance stabilizing transformation in DESeq2 R package (Love et al., 2014), principal component analysis was performed to visualize the clustering of samples Using the 250 most variable genes, unsupervised hierarchical clustering based on Euclidean distance was performed by the pheatmap R package. Differential expression analysis (BMEM vs. DF 12) was performed using the DESeq2 R package (Love et al., 2014) accounting for the matched pair design, cell passage, and an interaction term assessing the gene expression change by cell passage. Results were shown by a volcano plot generated using the ggplot2 R package. Genes with adjusted P-value <0.05 corrected for multiple testing using the Benjamini Hochberg method were considered as differentially expressed. Ingenuity Pathway Analysis (IPA) was performed to identify canonical pathways based on the differentially expressed genes (Krämer et al., 2014). This was used to identify differences in pathways related to biological processes that were relevant between our samples.

Statistical methods: Data were analyzed in Excel or R and graphed in GraphPad Prism 9.5. Detailed statistical information is included in the corresponding figure legends. Data are presented as mean±SEM. Data were checked for normal distribution and comparisons were conducted via an unpaired two-tailed Student's t test with significant differences defined as *p<0.05, **p<0.01, ***p<0.005, and ****p<0.0001. No statistical methods were used to predetermine sample size. The experiments were not randomized, and the investigators were not blinded to allocation during experiments and outcome assessment.

TABLE 4
List of materials and reagents

| | Catalog | |
|---|---|---|
| Component | Number | Supplier |
| Recombinant human Insulin | A11382ij | Gibco |
| AA2P | 321-44823 | Wako |
| Optiferrin | 777TRF029-10G | InVitria |
| Sodium Selenite | S5261 | Sigma |
| Recombinant human TGFB3 | Qk054-0100 | Qkine |
| Recombinant human NRG1 | 100-46-500ug | Shenodoah |
| | | Biotechnology |
| Matrigel | 354230 | Corning |
| Resazurin sodium salt | B21187.06 | Thermo |
| CHIR99021 | C-6556 | LC Labs |
| RPMI 1640 | 10-040-CM | Corning |
| Bovine serum albumin (fatty | A0100 | GenDEPOT |
| acid-free) | | |
| Wnt-C59 | orb181132 | Biorbyt |
| Liberase TH | 5401151001 | Roche |
| VEGFA₁₆₅ | 100-20 | Peprotech |
| Forskolin | F-9929 | LC Labs |
| Accutase | 25058Cl | Corning |
| PDGF-BB | 100-14B | Peprotech |
| Activin A | 120-14E | Peprotech |
| DMEM/F12 | 10-090-CV | Corning |
| Neurobasal Medium | 21103-049 | Gibco |
| L-glutamine | G3126 | Sigma |
| N2 Supplement | 17502048 | Gibco |
| B27 Supplement minus vitamin A | 12587010 | Gibco |
| SB431542 | S-7800 | LC Labs |
| LDN-193189 | HY-12071 | MedChemExpress |
| B27 minus insulin | A1895601 | Gibco |
| BMP4 | 120-05ET | Peprotech |
| DMSO | BP231-100 | Fisher |
| HGF | 100-39H | Peprotech |
| Synthemax II-SC | 3535 | Corning |
| rhVTN-N | A14700 | Gibco |
| ECMatrix-511 E8 Laminin | CC160 | Sigma |
| Substrate | | |
| Cultrex UltiMatrix Reduced | BME001-05 | Bio-Techne |
| Growth Factor BME | | |
| Geltrex | A14132-02 | Gibco |
| Bovine serum albumin | J64752 | Alfa Aesar |
| 20% Paraformaldehyde solution | 15713-S | Electron |
| | | Microscopy |
| | | Sciences |
| TrypLE Express | 12604-013 | Gibco |
| 0.5 mM EDTA | 46-034-Cl | Corning |
| Tween 20 | BP337-100 | Fisher |
| Thiazovivin | T9753 | LC Labs |

### REFERENCES

Ackermann, T., and Tardito, S. (2019). Cell culture medium formulation and its implications in cancer metabolism. Trends Cancer 5, 329-332.
Amit, M., Carpenter, M. K., Inokuma, M. S., Chiu, C. P., Harris, C. P., Waknitz, M. A., Itskovitz-Eldor, J., and Thomson, J. A. (2000). Clonally derived human embryonic stem cell lines maintain pluripotency and proliferative potential for prolonged periods of culture. Dev. Biol. 227, 271-278.
Bardy, C., van den Hurk, M., Eames, T., Marchand, C., Hernandez, R. V., Kellogg, M., Gorris, M., Galet, B., Palomares, V., Brown, J., et al. (2015). Neuronal medium that supports basic synaptic functions and activity of human neurons in vitro. Proc. Natl. Acad. Sci. USA 112, E2725-E2734.
Bubber, P., Ke, Z. J., and Gibson, G. E. (2004). Tricarboxylic acid cycle enzymes following thiamine deficiency. Neurochem. Int. 45, 1021-1028.
Burridge, P. W., Matsa, E., Shukla, P., Lin, Z. C., Churko, J. M., Ebert, A. D., Lan, F., Diecke, S., Huber, B., Mordwinkin, N. M., et al. (2014). Chemically defined generation of human cardiomyocytes. Nat. Methods 11, 855-860.
Cantor, J. R., Abu-Remaileh, M., Kanarek, N., Freinkman, E., Gao, X., Louissaint, A., Jr., Lewis, C. A., and Sabatini, D. M. (2017). Physiologie medium rewires cellular metabolism and reveals uric acid as an endogenous inhibitor of UMP synthase. Cell 169, 258-272.e17.
Chen, G., Gulbranson, D. R., Hou, Z., Bolin, J. M., Ruotti, V., Probasco, M. D., Smuga-Otto, K., Howden, S. E., Diol, N. R., Propson, N. E., et al. (2011). Chemically defined conditions for human iPSC derivation and culture. Nat. Methods 8, 424-429.
Cieciura, S. J., Marcus, P. I., and Puck, T. T. (1956). Clonal growth in vitro of epithelial cells from normal human tissues. J. Exp. Med. 104, 615-628.
Dulbecco, R., and Freeman, G. (1959). Plaque production by the polyoma virus. Virology 8, 396-397.
Eagle, H. (1955a). The minimum vitamin requirements of the L and HeLa cells in tissue culture, the production of specific vitamin deficiencies, and their cure. J. Exp. Med. 102, 595-600.
Eagle, H. (1955b). The specific amino acid requirements of a hu-man carcinoma cell (Stain HeLa) in tissue culture. J. Exp. Med. 102, 37-48.
Eagle, H. (1955c). The specific amino acid requirements of a mammalian cell (strain L) in tissue culture. J. Biol. Chem. 2/4, 839-852.
Eagle, H. (1956). The salt requirements of mammalian cells in tis-sue culture. Arch. Biochem. Biophys. 67, 356-366.
Eagle, H., Oyama, V. I., Levy, M., and Freeman, A. E. (1957). Myo-Inositol as an essential growth factor for normal and malignant hu-man cells in tissue culture. J. Biol. Chem. 226, 191-205.
Eagle, H., Oyama, V. I., Levy, M., Horton, C. L., and Fleischman, R. (1956). The growth response of mammalian cells in tissue culture to L-glutamine and L-glutamic acid. J. Biol. Chem. 278, 607-616.
Fisher, H. W., Puck, T. T., and Sato, G. (1959). Molecular growth requirements of single mammalian cells. III. Quantitative colonial growth of single S3 cells in a medium containing synthetic small molecular constituents and two purified protein fractions. J. Exp. Med. 109, 649-660.
Ham, R. G. (1962). Clonal growth of diploid Chinese hamster cells in a synthetic medium supplemented with purified protein fractions. Exp. Cell Res. 28, 489-500.
Ham, R. G. (1963). An improved nutrient solution for diploid Chinese hamster and human cell lines. Exp. Cell Res. 29, 515-526.
Ham, R. G. (1965). Clonal growth of mammalian cells in a chemically defined, synthetic medium. Proc. Natl. Acad. Sci. USA 53, 288-293.
Kuo, H. H., Gao, X., DeKeyser, J. M., Fetterman, K. A., Pinheiro, E. A., Weddle, C. J., Fonoudi, H., Orman, M. V., Romero-Tejeda, M., Jouni, M., et al. (2020) Negligible-cost and weekend-free chemically defined human iPSC culture. Stem Cell Rep. 14, 256-270.
Lockart, R. Z., Jr., and Eagle, H. (1959). Requirements for growth of single human cells. Science/29, 252-254.
Ludwig, T. E., Levenstein, M. E., Jones, J. M., Berggren, W. T.,
Mitchen, E. R., Frane, J. L., Crandall, L. J., Daigh, C. A., Conard, K. R., Piekarczyk, M. S., et al. (2006). Derivation of human embryonic stem cells in defined conditions. Nat. Biotechnol. 24, 185-187.
McLimans, W. F., Davis, E. V., Glover, F. L., and Rake, G. W. (1957). The submerged culture of mammalian cells; the spinner culture. J. Immunol. 79, 428-433.
Messmer, T., von Meyenn, F., Savino, A., Santos, F., Mohammed, H., Lun, A. T. L., Marioni, J. C., and Reik, W. (2019). Transcriptional heterogeneity in naive and primed human pluripotent stem cells at single-cell resolution. Cell Rep. 26, 815-824.e4.
Puck, T. T., Marcus, P. I., and Cieciura, S. J. (1956). Clonal growth of mammalian cells in vitro; growth characteristics of colonies from single HeLa cells with and without a feeder layer. J. Exp. Med. 103, 273-283.
Ramos-Ibeas, P., Barandalla, M., Colleoni, S., and Lazzari, G. (2017). Pyruvate antioxidant roles in human fibroblasts and embryonic stem cells. Mol. Cell. Biochem. 429, 137-150.
Sato, G., Fisher, H. W., and Puck, T. T. (1957). Molecular growth requirements of single mammalian cells. Science 126, 961-964.
Schulz, T. C., Noggle, S. A., Palmarini, G. M., Weiler, D. A., Lyons, I. G., Pensa, K. A., Meedeniya, A. C. B., Davidson, B. P., Lambert, N. A., and Condie, B. G. (2004). Differentiation of human embryonic stem cells to dopaminergic neurons in serum-free suspension culture. Stem Cell. 22, 1218-1238.
Tardito, S., Oudin, A., Ahmed, S. U., Fack, F., Keunen, O., Zheng, L., Miletic, H., Sakariassen, P. Ø., Weinstock, A., Wagner, A., et al. (2015). Glutamine synthetase activity fuels nucleotide biosynthesis and supports growth of glutamine-restricted glioblastoma. Nat. Cell Biol. 17, 1556-1568.
Thompson, O., von Meyenn, F., Hewitt, Z., Alexander, J., Wood, A., Weightman, R., Gregory, S., Krueger, F., Andrews, S., Barbaric, I., et al. (2020). Low rates of mutation in clinical grade human pluripotent stem cells under different culture conditions. Nat. Commun. 11, 1528.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Vande Voorde, J., Ackermann, T., Pfetzer, N., Sumpton, D., Mackay, G., Kalna, G., Nixon, C., Blyth, K., Gottlieb, E., and Tardito, S. (2019). Improving the metabolic fidelity of cancer models with a physiological cell culture medium. Sci. Adv. 5, eaau 7314.
Chen, G., Gulbranson, D. R., Yu, P., Hou, Z., and Thomson, J. A. (2012). Thermal stability of fibroblast growth factor protein is a determinant factor in regulating self-renewal, differentiation, and reprogramming in human pluripotent stem cells. Stem Cells 30, 623-630.
Chen, Y., Chen, Y., Shi, C., Huang, Z., Zhang, Y., Li, S., Li, Y., Ye, J., Yu, C., Li, Z., et al. (2018). SOAPnuke: a MapReduce acceleration-supported software for integrated quality control and preprocessing of high-throughput sequencing data. Gigascience 7, 1-6.
Chou, B. K., Gu, H., Gao, Y., Dowey, S. N., Wang, Y., Shi, J., Li, Y., Ye, Z., Cheng, T., and Cheng, L. (2015). A facile method to establish human induced pluripotent stem cells from adult blood cells under feeder-free and xenofree culture conditions: a clinically compliant approach. Stem Cells Transl Med 4, 320-332.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Fujie, Y., Fusaki, N., Katayama, T., Hamasaki, M., Soejima, Y., Soga, M., Ban, H., Hasegawa, M., Yamashita, S., Kimura, S., et al. (2014). New type of Sendai virus vector provides transgene-free iPS cells derived from chimpanzee blood. PLOS One 9, e113052.
Fusaki, N., Ban, H., Nishiyama, A., Saeki, K., and Hasegawa, M. (2009). Efficient induction of transgene-free human pluripotent stem cells using a vector based on Sendai virus, an RNA virus that does not integrate into the host genome. Proc Jpn Acad Ser B Phys Biol Sci 85, 348-362.
Krämer, A., Green, J., Pollard, J., Jr., and Tugendreich, S. (2014). Causal analysis approaches in Ingenuity Pathway Analysis. Bioinformatics 30, 523-530.
Kuo, H. H., Gao, X., DeKeyser, J. M., Fetterman, K. A., Pinheiro, E. A., Weddle, C. J., Fonoudi, H., Orman, M. V., Romero-Tejeda, M., Jouni, M., et al. (2020). Negligible-Cost and Weekend-Free Chemically Defined Human iPSC Culture. Stem Cell Reports 14, 256-270.
Li, B., and Dewey, C. N. (2011). RSEM: accurate transcript quantification from RNA-Seq data with or without a reference genome. BMC Bioinformatics/2, 323.
Love, M. I., Huber, W., and Anders, S. (2014). Moderated estimation of fold change and dispersion for RNA-seq data with DESeq2. Genome Biol 15, 550.
Magdy, T., Jouni, M., Kuo. H. H., Weddle, C. J., Lyra-Leite, D., Fonoudi, H., Romero-Tejeda, M., Gharib, M., Javed, H., Fajardo, G., et al. (2022). Identification of Drug Transporter Genomic Variants and Inhibitors That Protect Against Doxorubicin-Induced Cardiotoxicity. Circulation 145, 279-294.
Pang, Z., Zhou, G., Ewald, J., Chang, L., Hacariz, O., Basu, N., and Xia, J. (2022). Using MetaboAnalyst 5.0 for LC-HRMS spectra processing, multi-omics integration and covariate adjustment of global metabolomics data. Nat Protoc 17, 1735-1761.
Patsch, C., Challet-Meylan, L., Thoma, E. C., Urich, E., Heckel, T., O'Sullivan, J. F., Grainger, S. J., Kapp, F. G., Sun, L., Christensen, K., et al. (2015). Generation of vascular endothelial and smooth muscle cells from human pluripotent stem cells. Nat Cell Biol 17, 994-1003.
Peters, D. T., Henderson, C. A., Warren, C. R., Friesen, M., Xia, F., Becker, C. E., Musunuru, K., and Cowan, C. A. (2016). Asialoglycoprotein receptor 1 is a specific cell-surface marker for isolating hepatocytes derived from human pluripotent stem cells. Development 143, 1475-1481.
Toyohara, T., Roudnicky, F., Florido, M. H. C., Nakano, T., Yu, H., Katsuki, S., Lee, M., Meissner, T., Friesen, M., Davidow, L. S., et al. (2020). Patient hiPSCs Identify Vascular Smooth Muscle Arylacetamide Deacetylase as Protective against Atherosclerosis. Cell Stem Cell 27, 147-157 e147.
Weinberg, S. E., Singer, B. D., Steinert, E. M., Martinez, C. A., Mehta, M. M., Martinez-Reyes, 1., Gao, P., Helmin, K. A., Abdala-Valencia, H., Sena, L. A., et al. (2019). Mitochondrial complex III is essential for suppressive function of regulatory T cells. Nature 565, 495-499.
Zhong, Q., Laco, F., Liao, M. C., Woo, T. L., Oh, S. K. W., and Chai, C. L. L. (2018). Influencing the Fate of Cardiac and Neural Stem Cell Differentiation Using Small Molecule Inhibitors of ALK5. Stem Cells Transl Med 7, 709-720.

We claim:

1. A media for culturing induced human pluripotent stem cells (iPSCs), wherein the media comprises components for culturing iPSCs, wherein the components comprise
a) a salt solution, comprising Calcium chloride (CaCl₂)), Iron(II) sulfate heptahydrate (FeSO4·7H2O), Magnesium sulfate (MgSO4), Potassium chloride (KCl), Sodium bicarbonate (NaHCO3), Sodium chloride (NaCl), Sodium phosphate monobasic monohydrate (NaH2PO4·H2O) and Zinc sulfate heptahydrate (ZnSO4·7H2O);
b) a vitamin solution comprising Biotin (B7), Choline bitartrate, D-Pantothenic acid hemicalcium salt (B5), Folic acid (B9), Nicotinamide (B3), Pyridoxine hydrochloride (B6), Riboflavin (B2), and myo-Inositol; and
c) an amino acid solution comprising Glycine, L-Arginine HCl, L-Cysteine HCl·H2O, L-Glutamine, L-Histidine HCl·H2O, L-Isoleucine, L-Leucine, L-Lysine HCl, L-Methionine, L-Phenylalanine, L-Threonine, L-Tryptophan, L-Tyrosine disodium salt hydrate, and L-Valine.

2. The media of claim 1, wherein the amino acid solution further comprises L-Alanine, L-Asparagine·H2O, L-Aspartic acid, L-Glutamic acid, L-Proline and L-Serine.

3. The media of claim 1 further comprising D-(+)-Glucose, Phenol red sodium salt, and sodium pyruvate.

4. The media of claim 1, wherein the salt solution does not comprise Copper (II) sulfate pentahydrate (CuSO4·5H2O), Iron (III) nitrate nonahydrate (Fe(NO3)3·9H2O), Magnesium chloride (MgCl2), or Sodium phosphate dibasic (Na2HPO4).

5. The media of claim 1, wherein the vitamin solution does not comprise choline chloride, Pyridoxal hydrochloride (B6), Thiamine hydrochloride (B1), or Vitamin B12.

6. The media of claim 1, wherein the amino acid solution does not comprise L-Cystine 2HCl or L-Histidine.

7. The media of claim 1, wherein the media does not comprise HEPES, Hypoxanthine, Linoleic acid, Lipoic acid, Phenol red, Putrescine dihydrochloride, or Thymidine.

8. The media of claim 1, wherein the media does not comprise any copper salt or linoleic acid.

9. The media of any of claim 1, wherein the media comprises the following components:
(a) amino acids comprising: Glycine; L-Alanine; L-Arginine; L-Asparagine; L-Aspartic acid, L-Cysteine; L-Glutamic Acid; L-Glutamine; L-Histidine HCL-H₂O; L-Isoleucine; L-Leucine, L-Lysine; L-Methionine; L-Phenylalanine; L-Proline, L-Serine, L-Threonine; L-Tryptophan; L-Tyrosine; and L-Valine;
(b) vitamins comprising: Biotin (B7); Choline bitartrate; D-Pantothenic acid (B5); Folic acid (B9); Nicotinamide (B3); Pyridoxine (B6); Riboflavin (B2); and myo-Inositol;
(c) inorganic salts comprising: Calcium chloride, Iron (II) sulfate heptahydrate, Magnesium sulfate; Potassium chloride; Sodium bicarbonate; Sodium chloride; Sodium phosphate monobasic monohydrate; and Zinc sulfate heptahydrate; and
(d) D-(+)-glucose; phenol red sodium salt; and sodium pyruvate.

10. The media of claim 1, wherein the media has an osmolarity of about 285-315 mOsm/L.

11. The media of claim 1, wherein the media has a pH of about 7.0-7.2.

12. The media of claim 1, wherein the Iron (II) sulfate heptahydrate (FeSO₄·7H₂O) is present in the media at a concentration of about 0.1-0.3 uM.

13. The media of claim 1, wherein the zinc sulfate heptahydrate is present in the media at a concentration of about 0.1-0.3 uM.

14. The media of claim 1 wherein the concentration is of L-Arginine is about 150-250 uM, the concentration of L-tryptophan is about 150-250 uM, and the concentration of L-glutamine is about 500-1500 uM.

15. The media of claim 1, wherein the concentration of biotin is about 0.001-01 uM, the concentration of folic acid is about 0.05-5 uM, the concentration of nicotinamide is about 0.01-5 uM, and the concentration of pyridoxine is about 0.05-10 uM.

16. The media of claim 1, wherein the CaCl₂ is present at a concentration of about 350-650 uM.

17. The media of claim 1, wherein the sodium pyruvate is present in the media at a concentration of about 600-900 uM.

18. The media of claim 9, wherein glycine is at a concentration of about 150-250 uM; L-Alanine is at a concentration of about 35-65 uM; L-Arginine is at a concentration of about 150-250 uM; L-Asparagine is at a concentration of about 35-65 uM; L-Cysteine is at a concentration of about 75-125 uM; L-Glutamic Acid is at a concentration of about 35-65 uM; L-Glutamine is at a concentration of about 800-1200 uM; L-Histidine is at a concentration of about 75-125 uM; L-Isoleucine is at a concentration of about 150-250 uM; L-Leucine is at a concentration of about 350-650 uM; L-Lysine is at a concentration of about 350-650 uM; L-Methionine is at a concentration of about 75-125 uM; L-Phenylalanine is at a concentration of about 75-125 uM; L-Threonine is at a concentration of about 350-650 uM; L-Tryptophan is at a concentration of about 150-250 uM, L-Tyrosine is at a concentration of about 150-250 uM; and L-Valine is at a concentration of about 150-250 uM.

19. The media of claim 1, wherein the media additionally comprises a supplement.

20. The media of claim 19, wherein the supplement comprises insulin, L-ascorbic acid, 2-phosphate trisodium salt, Oryza sativa-derived recombinant human transferrin, sodium selenite, recombinant human FGF2-G3, recombinant human TGFb3, and recombinant human NRG1.

21. The media of claim 20, wherein the human insulin is in a concentration of about 2.5-10 mg/mL, L-ascorbic acid 2-phosphate trisodium salt is at a concentration of about 150-250 mg/mL, recombinant human transferrin is at a concentration of about 2.5-10 mg/mL, sodium selenite is at concentration of about 10-30 ng/ml, recombinant human FGF2-G3 is at about a concentration of about 30-50 ng/mL, and/or recombinant human TGFb3 is at a concentration of about 0.05-0.5 ng/mL, recombinant human NRG1 is at a concentration of about 05-0.5 ng/mL.

22. A method for culturing induced pluripotent stem cells (IPSCs), the method comprising seeding the iPSCs in the media of claim 1, and culturing the iPSCs under conditions suitable for culturing the iPSCs.

23. The method of claim 22, wherein the media is not changed during culture of the iPSCs.

24. The method of claim 22, wherein the cultured iPSCs express one or more markers at a higher level than iPSCs cultured in Dulbecco's modified Eagle Media (DMEM), the markers selected from the group consisting of KDR, SPRY4, GDF3, NODAL, NANOG, POU5F1, TDGF1, ZIC2 DUSP6, ZDHHC22, NEFM, THY1, FAT3, STC1, KLHL4, PLA2G3, PTPRZ1, SOX11 SSEA4, TRA-1-60, and TRA-1-81.

25. The method of claim 22, wherein the iPSCs are further differentiated into cardiomyocytes, endothelial, vascular smooth muscle, neural and hepatocyte-like cells.
